SEQUENCE LISTING (30 sequences)

SEQ ID NO: 1 - protein, 113 aa, Artificial Sequence
  source: /note=“Description of Artificial Sequence Synthetic polypeptide”
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNF

SEQ ID NO: 2 - protein, 10 aa, Artificial Sequence
  source: /note=“Description of Artificial Sequence Synthetic peptide”
GGGSSGGGSG

SEQ ID NO: 3 - protein, 12 aa, Artificial Sequence
  source: /note=“Description of Artificial Sequence Synthetic peptide”
ESKYGPPCPPCP

SEQ ID NO: 4 - protein, 12 aa, Homo sapiens
ESKYGPPCPSCP

SEQ ID NO: 5 - protein, 22 aa, Artificial Sequence
  source: /note=“Description of Artificial Sequence Synthetic peptide”
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, Artificial Sequence
  source: /note=“Description of Artificial Sequence Synthetic polypeptide”
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  source: /note=“Description of Artificial Sequence Synthetic polypeptide”
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  source: /note=“Description of Artificial Sequence Synthetic polypeptide”
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  source: /note=“Description of Artificial Sequence Synthetic polypeptide”
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 323 aa, Artificial Sequence
  source: /note=“Description of Artificial Sequence Synthetic polypeptide”
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Artificial Sequence
  source: /note=“Description of Artificial Sequence Synthetic peptide”
LEGGGEGRGSLLTCGDVEENPGPR

SEQ ID NO: 28 - protein, 354 aa, Artificial Sequence
  source: /note=“Description of Artificial Sequence Synthetic polypeptide”
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 21 aa, Homo sapiens
PFFFCCFIAVAMGIRFIIMVA

SEQ ID NO: 30 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

(19) United States
(12) Patent Application Publication
(10) Pub. No.: US 2023/0364138 A1
(43) Pub. Date: Nov. 16, 2023

(54) ENGINEERED T CELLS FOR EXPRESSION OF CHIMERIC ANITGEN RECEPTORS

(71) Applicants: City of Hope, Duarte, CA (US); The Regents of the University of California, Oakland, CA (US)

(72) Inventors: Christine E. Brown, Duarte, CA (US); Dongrui Wang, Duarte, CA (US); Jeremy Rich, La Jolla, CA (US); Qi Xie, Hangzhou (CN); Briana Prager, Oakland, CA (US)

(21) Appl. No.: 18/038,101

(22) PCT Filed: Nov. 23, 2021
(86) PCT No.: PCT/US2021/060654

§ 371 Date: May 22, 2023

Related U.S. Application Data
(60) Provisional application No. 63/117,439, filed on Nov. 23, 2020.

(51) Int. Cl. A61K 35/17; C12N 15/113; C12N 9/22; C07K 14/47; C07K 14/725

(52) U.S. Cl. CPC A61K 35/17; C12N 15/1138; C12N 9/22; C07K 14/4703; C07K 14/4705; C07K 14/7051; C12N 2310/20

(57) ABSTRACT

Disclosed herein, inter alia, are methods of making and using engineered T cells useful for expressing a chimeric antigen receptor (CAR) targeted to a cell surface protein (e.g., a CAR targeted to IL13Rα2, which is highly expressed on glioblastoma cells).

### CLAIM OF PRIORITY

This application claims the benefit of U.S. Provisional Application Ser. No. 63/117,439, filed on Nov. 23, 2020. The entire contents of the foregoing are incorporated herein by reference.

### BACKGROUND

Glioblastoma (GBM) ranks as one of the most lethal of human cancers with current therapy offering only palliation. Standard-of-care therapy consisting of maximal surgical resection followed by combined radiation and chemotherapy extends median survival by less than 3 months. The activation of anti-tumor immune responses may provide new opportunities to augment tumor control. As such, immunotherapies have been extensively investigated with positive results in preclinical studies, yet broad antitumor efficacy has not occurred in patients (1). The adoptive transfer of chimeric antigen receptor (CAR) engineered T cells has shown promising clinical activity in a subset of cancers, particularly B cell malignancies (2,3). To target GBM, CAR T cells have been engineered to recognize selected tumor antigens and have demonstrated cytolytic activity against GBM cells, including GBM stem cells (GSCs) (4-6). In patients with GBM, CAR T cell therapies have shown early evidence of activity, clinical feasibility, and safety (7-10). However, the overall outcomes of CAR T cell treatment remain unsatisfactory, prompting efforts to enhance the antitumor potency of GBM-targeting CAR T cells (11,12). The functional potentiation of CAR T cells, while attractive due to the modifiable nature of these cells, requires a comprehensive understanding of the molecular events regulating CAR T cell activation, exhaustion and tumor-induced immune suppression (11,13).

Aside from CAR recognition of tumor antigens, the complicated and dynamic interaction between CAR T cells and their target tumor cells remains poorly characterized. Thus, there is a need for new strategies to further enhance CAR T cell potency.

Gene editing using the clustered randomly interspersed short palindromic repeats (CRISPR)-Cas9 is a promising approach to enhance cancer immunotherapy (14). Directed CRISPR-Cas9 gene knockout of checkpoint and other immune-regulatory receptors have shown utility for adoptive T cell therapy (15,16); however, this approach has focused on a limited set of known pathways. By contrast, large CRISPR-knockout screens are an effective platform for unbiased target discovery and have been successfully used to identify genes in tumor cells which when deleted synergize with various types of immunotherapeutics (17-19). CRISPR screens in T cells identified modulators of TCR activation in response to stimulation with CD3/CD28 agonistic beads, viruses, or tumor cells (20-22). Although CAR constructs are synthetic TCR-like receptors incorporating CD3ζ and costimulatory domains, the molecular events are not identical between TCR and CAR T cell activation signaling pathways (23).

### SUMMARY

Described below are genetically modified (edited) T cells having a disruption in one or more specific genes. The engineered T cells are useful for expressing a chimeric antigen receptor (CAR) targeted to a cell surface protein (e.g., a CAR targeted to IL13Ra2, which is highly expressed on glioblastoma cells). The engineered T cells having one or more or the gene disruptions described herein can be used to create CAR T cells having increased efficacy compared to otherwise identical CART T cells that lack the specific gene disruption.

The edited cells have reduced expression of one or more of: Transducin-Like Enhancer of Split 4 (TLE4), Transmembrane Protein 184B (MEM184B), a Eukaryotic Translation Initiation Factor 5A-1 (EIF5A) or Ikaros Family Zinc Finger Protein 2 (IKZF2). Editing of these genes to reduce expression (e.g., knockdown of expression or knockout of expression) can be achieved by generating of indels that result in disruption of a target gene, for example, reduction or elimination of gene expression and or function.

Described herein is a population of engineered human T cells, wherein the engineered human T cells comprise: a disrupted Transducin-Like Enhancer of Split 4 (TLE4) gene, a disrupted Transmembrane Protein 184B (MEM184B) gene, a disrupted Eukaryotic Translation Initiation Factor 5A-1 (EIF5A) gene or a disrupted Ikaros Family Zinc Finger Protein 2 (IKZF2) gene.

In various embodiments: the disrupted TLE4 gene comprises an insertion of at least 10 contiguous nucleotides into SEQ ID NO: D1; the disrupted MEM184B gene comprises an insertion of at least 10 contiguous nucleotides into SEQ ID NO: D2; the disrupted EIF5A gene comprises an insertion of at least 10 contiguous nucleotides into SEQ ID NO: D3; the disrupted IKZF2 gene comprises a deletion of at least 10 contiguous nucleotides of SEQ ID NO: D4; the disrupted TLE4 gene comprises a deletion of at least 10 contiguous nucleotides of SEQ ID NO: D1; the disrupted MEM184B gene comprises a deletion of at least 10 contiguous nucleotides of SEQ ID NO: D2; the disrupted EIF5A gene comprises a deletion of at least 10 contiguous nucleotides of SEQ ID NO: D3;m the disrupted IKZF2 gene comprises a deletion of at least 10 contiguous nucleotides of SEQ ID NO: D4; at least 75%, at least 80%, at least 85%, at least 90%, or at least 95% of the engineered T cells do not express a detectable level of TLE4; at least 75%, at least 80%, at least 85%, at least 90%, or at least 95% of the engineered T cells do not express a detectable level of MEM184B; at least 75%, at least 80%, at least 85%, at least 90%, or at least 95% of the engineered T cells do not express a detectable level of EIF5A; and at least 75%, at least 80%, at least 85%, at least 90%, or at least 95% of the engineered T cells do not express a detectable level of KZF2.

Also disclosed is a population of engineered T cells wherein the disrupted gene is disrupted by a nucleic acid encoding a chimeric antigen receptor.

In some case, at least 30% of the T cells comprises a nucleic acid molecule comprising a nucleotide sequence encoding a chimeric antigen receptor (CAR) wherein the chimeric antigen receptor comprises a targeting domain, a spacer, a transmembrane domain, a co-stimulatory domain, and a CD3 (signaling domain. In various cases: the targeting domain comprises a scFv that selectively binds a tumor cell antigen; the targeting domain comprises a ligand for a cell surface receptor; the nucleic acid molecule encoding the CAR is an mRNA.

Also described is a method for producing an engineered T cell, the method comprising: (a) delivering to a T cell: a RNA-guided nuclease, a gRNA targeting a TLE4 gene, a EMM1848 gene, or a KZF2 gene, a vector comprising a donor template that comprises a nucleic acid encoding a CAR; and (b) producing an engineered T cell suitable for allogeneic transplantation.

In some cases, the editing can include Insertion of a nucleic acid encoding a CAR into the disrupted genomic loci by using guide RNA/Cas9 to induce a double stranded break that is repaired by HDR using a donor template with homology around the cut site. Thus, the methods described herein can be used to knock-in a nucleic acid encoding a chimeric antigen receptor (CAR) in or near a locus of a target gene by permanently deleting at least a portion of the target gene and inserting a nucleic acid encoding the CAR. The CARs described herein include a targeting domain, a spacer, a transmembrane domain, a co-stimulatory domain, and a CD3 (signaling domain.

Provided herein are methods to DNA double stranded breaks (DBSs) that induce small insertions or deletions in a target gene resulting in the disruption (e.g., reduction or elimination of gene expression and/or function) of the target gene. Also described are methods to create and/or permanently delete within or near the target gene and to insert a nucleic acid construct encoding a CAR construct in the gene by inducing a double stranded break with Cas9 and a sgRNA in a target sequence (or a pair of double stranded breaks using two appropriate sgRNAs), and to provide a donor DNA template to induce Homology-Directed Repair (HDR). In some embodiments, the donor DNA template can be a short single stranded oligonucleotide, a short double stranded oligonucleotide, a long single or double stranded DNA molecule. These methods use gRNAs and donor DNA molecules for each target. In some embodiments, the donor DNA is single or double stranded DNA having homologous arms to the corresponding region. In some embodiments, the homologous arms are directed to the nuclease-targeted region of a gene selected from the group consisting of: Transducin-Like Enhancer of Split 4 (TLE4), Transmembrane Protein 184B (MEM184B), a Eukaryotic Translation Initiation Factor 5A-1 (EIF5A) or Ikaros Family Zinc Finger Protein 2 (IKZF2).

Provided herein are cellular methods (e.g., ex vivo or in vivo) methods for using genome engineering tools to create permanent changes to the genome by: 1) creating DSBs to induce small insertions, deletions or mutations within or near a target gene, 2) deleting within or near the target gene or other DNA sequences that encode regulatory elements of the target gene and inserting, by HDR, a nucleic acid encoding a knock-in CAR construct within or near the target gene or other DNA sequences that encode regulatory elements of the target gene, or 3) creating DSBs within or near the target gene and inserting a nucleic acid construct within or near the target gene by HDR. Such methods use endonucleases, such as CRISPR-associated (Cas9, Cpfl and the like) nucleases, to permanently delete one or more or exons or portions of exons of the target genes.

### Design of Chimeric Antigen Receptors for Expression by Engineered T Cells

A very large number of CAR have are known. The engineered T cells described herein can be used to express any selected CAR.

### Targeting Region

The targeting region comprises a ligand for a cell-surface receptor or a scFv targeted to a cell surface molecule.

In the case of a CAR targeted to IL13Ra, the targeting region can comprises or consist of the amino acid sequence GPVPPSTALRYLIEELVNITQNQKAPLCNGSMVWSINLTAGMYCAALESLINVSGCS AIEKTQRMLSGFCPHKVSAGQFSSLHVRDTKIEVAQFVKDLLLHLKKLFREGRFNF (SEQ ID NO:1), which is a variant of human IL13. A suitable CAR targeted to IL13Ra is described in U.S. Pat. No. 9,914,909.

### Spacer Region

The CAR or polypeptide described herein can include a spacer located between the CD45 targeting domain (i.e., a CD45 targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| S228P) | | |
| IgG4 hinge | 12 aa | ESKYGPPCPSCP (SEQ ID NO: 4) |
| IgG4 hinge (S228P)+ | 22 aa | ESKYGPPCPPCPGGGSSGGGSG (SEQ ID NO: 5) |
| linker | | |
| CD28 hinge | 39 aa | IEVMYPPPYLDNEKSNGTIIHVKGKHLCPSPLFPG |
| | | PSKP (SEQ ID NO: 6) |
| CD8 hinge-48 aa | 48 aa | AKPTTTPAPRPPTPAPTIASQPLSLRPEACRPAAG |
| | | GAVHTRGLDFACD (SEQ ID NO: 7) |
| CD8 hinge-45 aa | 45 aa | TTTPAPRPPTPAPTIASQPLSLRPEACRPAAGGA |
| | | VHTRGLDFACD (SEQ ID NO: 8) |
| IgG4 (HL-CH3) | 129 aa | ESKYGPPCPPCPGGGSSGGGSGGQPREPQVYT |
| Also called IgG4 | | LPPSQEEMTKNQVSLTCLVKGFYPSDIAVEWESN |
| (HL-ΔCH2) | | GQPENNYKTTPPVLDSDGSFFLYSRLTVDKSRW |
| (includes S228P in hinge) | | QEGNVFSCSVMHEALHNHYTQKSLSLSLGK |
| | | (SEQ ID NO: 9) |
| IgG4 | 229 aa | ESKYGPPCPSCPAPEFEGGPSVFLFPPKPKDTLM |
| (L235E, N297Q) | | ISRTPEVTCVVVDVSQEDPEVQFNWYVDGVEVH |
| | | NAKTKPREEQFQSTYRVVSVLTVLHQDWLNGKE |
| | | YKCKVSNKGLPSSIEKTISKAKGQPREPQVYTLPP |
| | | SQEEMTKNQVSLTCLVKGFYPSDIAVEWESNGQ |
| | | PENNYKTTPPVLDSDGSFFLYSRLTVDKSRWQE |
| | | GNVFSCSVMHEALHNHYTQKSLSLSLGK (SEQ ID |
| | | NO: 10) |
| IgG4 | 229 aa | ESKYGPPCPPCPAPEFEGGPSVFLFPPKPKDTLM |
| (S228P, L235E, N297Q) | | ISRTPEVTCVVVDVSQEDPEVQFNWYVDGVEVH |
| | | NAKTKPREEQFQSTYRVVSVLTVLHQDWLNGKE |
| | | YKCKVSNKGLPSSIEKTISKAKGQPREPQVYTLPP |
| | | SQEEMTKNQVSLTCLVKGFYPSDIAVEWESNGQ |
| | | PENNYKTTPPVLDSDGSFFLYSRLTVDKSRWQE |
| | | GNVFSCSVMHEALHNHYTQKSLSLSLGK (SEQ ID |
| | | NO: 11) |
| IgG4 (CH3) | 107 aa | GQPREPQVYTLPPSQEEMTKNQVSLTCLVKGFY |
| Also called IgG4 (ΔCH2) | | PSDIAVEWESNGQPENNYKTTPPVLDSDGSFFLY |
| | | SRLTVDKSRWQEGNVFSCSVMHEALHNHYTQKS |
| | | LSLSLGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ACH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3). The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTP PVLDSDGSFFLYSRLTVDKSRWQEGNVFSCSVMHEALHNHYTQKSLSLSLGK (SEQ ID NO:12). Thus, the entire linker/spacer region can comprise the sequence: ESKYGPPCPPCPGGGSSGGGSGGQPREPQVYTLPPSQEEMTKNQVSLTCLVKGF YPSDIAVEWESNGQPENNYKTTPPVLDSDGSFFLYSRLTVDKSRWQEGNVFSCSV MHEALHNHYTQKSLSLSLGK (SEQ ID NO:11). In some cases, the spacer has 1, 2, 3, 4, or 5 single amino acid changes (e.g., conservative changes) compared to SEQ ID NO:11. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the CAR. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID |
| | | | NO: 13 |
| CD28 | NM_006139 | 27 aa | FWVLVVVGGVLACYSLLVTVAFIIFWV |
| | | | (SEQ ID NO: 14) |
| CD28(m) | NM_006139 | 28 aa | MFWVLVVVGGVLACYSLLVTVAFIIFWV |
| | | | (SEQ ID NO: 15) |
| CD4 | M35160 | 22 aa | MALIVLGGVAGLLLFIGLGIFF (SEQ ID |
| | | | NO: 16) |
| CD8tm | NM_001768 | 21 aa | IYIWAPLAGTCGVLLLSLVIT (SEQ ID |
| | | | NO: 17) |
| CD8tm2 | NM_001768 | 23 aa | IYIWAPLAGTCGVLLLSLVITLY (SEQ ID |
| | | | NO: 18) |
| CD8tm3 | NM_001768 | 24 aa | IYIWAPLAGTCGVLLLSLVITLYC (SEQ |
| | | | ID NO: 19) |
| 41BB | NM_001561 | 27 aa | IISFFLALTSTALLFLLFF LTLRFSVV |
| | | | (SEQ ID NO: 20) |
| NKG2D | NM_007360 | 21 aa | PFFFCCFIAVAMGIRFIIMVA (SEQ ID |
| | | | NO: X) |

### Costimulatory Domain

The costimulatory domain can be any domain that is suitable for use with a CD3ζ signaling domain. In some cases the co-signaling domain is a 4-1 BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1 BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD37 Domain and Examples of Costimulatory Domains

| Name | Accession | Length | Sequence |
|---|---|---|---|
| Y | J04132.1 | 113 aa | RVKFSRSADAPAYQQGQNQLYNELNLG |
| | | | RREEYDVLDKRRGRDPEMGGKPRRKNP |
| | | | QEGLYNELQKDKMAEAYSEIGMKGERR |
| | | | RGKGHDGLYQGLSTATKDTYDALHMQA |
| | | | LPPR (SEQ ID NO: 21) |
| CD28 | NM_006139 | 42 aa | RSKRSRLLHSDYMNMTPRRPGPTRKHY |
| | | | QPYAPPRDFAAYRS (SEQ ID NO: 22) |
| CD28gg* | NM_006139 | 42 aa | RSKRSRGGHSDYMNMTPRRPGPTRKH |
| | | | YQPYAPPRDFAAYRS (SEQ ID NO: 23) |
| 41BB | NM_001561 | 142 aa | KRGRKKLLYIFKQPFMRPVQTTQEEDGC |
| | | | SCRFPEEEEGGCEL (SEQ ID NO: 24) |
| OX40 | NM_003327 | 42 aa | ALYLLRRDQRLPPDAHKPPGGGSFRTPI |
| | | | QEEQADAHSTLAKI (SEQ ID NO: 25) |
| 2B4 | NM_016382 | 120 aa | WRRKRKEKQSETSPKEFLTIYEDVKDLK |
| | | | TRRNHEQEQTFPGGGSTIYSMIQSQSSA |
| | | | PTSQEPAYTLYSLIQPSRKSGSRKRNHS |
| | | | PSFNSTIYEVIGKSQPKAQNPARLSRKEL |
| | | | ENFDVYS (SEQ ID NO: Y) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1 BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1 BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1 BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

In some cases, the CAR can include two co-stimulatory domains, e.g., CD28 and 41 BB (in either order); OX40 and 41 BB (in either order); or CD28 and OX40 (in either order). Where two co-stimulatory domains are present, a spacer of 4-20 amino acids can be located between the two co-stimulatory domains.

Other co-stimulatory domains that can be used include: CD27, CD30, CD40, PD-1, ICOS, CD2, CD7, LIGHT, NKG2C, B7-H3, CDS, ICAM-1, GITR, BAFFR, HVEM (LIGHTR), SLAMF7, NKp80 (KLRF1), CD160, CD19. CD4, CD8a, CD8, IL2RP, IL2Ry, IL7Ra, ITGA4, VLA1, CD49a, ITGA4, IA4, CD49D, ITGA6, VLA-6, CD49f, ITGAD, CD11d, ITGAE. CD103, ITGAL, CDIIa, LFA-1, ITGAM, CDI Ib, ITGAX, CDI Ic. ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229). CD160 (BY55), PSGL1, CD100 (SEMA4D), CD69, SLAMF6 (NTB-A, LyI08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

### CD3Z Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR (SEQ ID NO:21). In some cases, the CD3ζ signaling has 1, 2, 3, 4 of 5 amino acid changes (preferably conservative) compared to SEQ ID NO:21.

### Truncated EGFR or CD19

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPV AFRGDSFTHTPPLDPQELDILKTVKEITGFLLIQAWPENRTDLHAFENLEIIRGRTKQ HGQFSLAVVSLNITSLGLRSLKEISDGDVIISGNKNLCYANTINWKKLFGTSGQKTKII SNRGENSCKATGQVCHALCSPEGCWGPEPRDCVSCRNVSRGRECVDKCNLLEG EPREFVENSECIQCHPECLPQAMNITCTGRGPDNCIQCAHYIDGPHCVKTCPAGVM GENNTLVWKYADAGHVCHLCHPNCTYGCTGPGLEGCPTNGPKIPSIATGMVGALL LLLWALGIGLFM (SEQ ID NO:28). In some cases, the truncated EGFR has 1, 2, 3, 4 of 5 amino acid changes (preferably conservative) compared to SEQ ID NO:28. Alternatively the CD3ζ signaling domain can be followed by a ribosomal skip sequence (e.g., LEGGGEGRGSLLTCGDVEENPGPR; SEQ ID NO:27) and a truncated CD19R (also called CD19t) having a sequence that is at least 90%, at least 95%, at least 98% identical to or identical to:

(SEQ ID NO: 26)
MPPPRLLFFLLFLTPMEVRPEEPLVVKVEEGDNAVLQCLKGTSDGPTQQ
LTWSRESPLKPFLKLSLGLPGLGIHMRPLAIWLFIFNVSQQMGGFYLCQ
PGPPSEKAWQPGWTVNVEGSGELFRWNVSDLGGLGCGLKNRSSEGPSSP
SGKLMSPKLYVWAKDRPEIWEGEPPCVPPRDSLNQSLSQDLTMAPGSTL
WLSCGVPPDSVSRGPLSWTHVHPKGPKSLLSLELKDDRPARDMWVMETG
LLLPRATAQDAGKYYCHRGNLTMSFHLEITARPVLWHWLLRTGGWKVSA
VTLAYLIFCLCSLVGILHLQRALVLRRKR

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the CAR lentiviral vector can act as suicide gene to ablate the CAR+ T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte, and most preferably an autologous T lymphocyte.

Various T cell subsets isolated from the patient can be transduced with a vector for CAR or polypeptide expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an CD45 CAR or CD45 polypeptide as well as a non-immunogenic surface marker for in vivo detection, ablation, and potential ex vivo selection. The activated/genetically modified CD45 central memory T cells can be expanded in vitro with IL-2/IL-15 and then cryopreserved. Additional methods of preparing CAR T cells can be found in PCT/US2016/043392. Methods for preparing T cell populations useful for producing engineered T cells are described in, for example, WO 2017/015490 and WO 2018/102761.

The CAR can be transiently expressed in a T cell population by an mRNA encoding the CAR. The mRNA can be introduced into the T cells by electroporation (Wiesinger et al. 2019 Cancers (Basel) 11:1198).

In some embodiments, a composition comprising the CAR T cells comprise one or more of helper T cells, cytotoxic T cells, memory T cells, naïve T cells, regulatory T cells, natural killer T cells, or combinations thereof. In some embodiments, a composition comprising the CAR T cells comprise CD3+, CD5+, CD7+, and TCRαβ+. In some embodiments, a composition comprising the CAR T cells comprise CD8+ CAR T cells are CD8αβ T cells, which have strong cytotoxicity against tumor cells in an antigen specific manner and can potently secret cytokines such as IFNγ. In some embodiments, CAR T cells have predominant homogenous TCR phenotype. In some embodiments, a composition comprising the CAR T cells comprise CD3+CD5+CD7+TCRαβ+CD8αβ+, CD3+CD5+CD7+TCRαβ+CD4+, CD62L+CD45RA+ stem memory T cells, CD62L-CD45RA-CD45RO+ effector memory T cells and CD62L-CD45RA+ effector T cells, and combinations thereof.

In some embodiments, a gene selected from: Transducin Like Enhancer of Split 4 (TLE4) gene, Transmembrane Protein 184B (MEM184B) gene, Eukaryotic Translation Initiation Factor 5A-1 (EIF5A) gene and Ikaros Family Zinc Finger Protein 2 (IKZF2) is knocked out, knocked down, mutated, or down regulated. Preferably, the gene is knocked down or knocked out by gene disruption, e.g., using methods described herein or other gene modification methods known in the art. In some embodiments, the genetic modification method comprises gene editing, homologous recombination, non-homologous recombination, RNA-mediated genetic modification, DNA-mediated genetic modification, zinc finger nucleases, meganucleases, TALEN, or CRISPR/CAS9. In some embodiments, the CRISPR/CAS9 system comprises a gRNA targeting an exon of one of the genes that is to be disrupted.

In some embodiments, a composition comprising CAR T cells or CAR NK cells described herein is administered locally or systemically. In some embodiments, a composition comprising CAR T cells or CAR NK cells described herein is administered by single or repeat dosing. In some embodiments, a composition comprising CAR T cells or CAR NK cells described herein is administered to a patient having a cancer, a pathogen infection, an autoimmune disorder, or undergoing allogeneic transplant.

In some embodiments, the engineered T cells express a CAR targeted to a cancer cell antigen. In some embodiments, the cancer is glioblastoma. In some embodiments, the cancer is selected from the group consisting of blood cancer, B cell leukemia, multiple myeloma, lymphoblastic leukemia (ALL), chronic lymphocytic leukemia, non-Hodgkin's lymphoma, ovarian cancer, prostate cancer, pancreatic cancer, lung cancer, breast cancer, and sarcoma, acute myeloid leukemia (AML).

The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### FIGURES

FIG. 1A-F. CRISPR-Cas9 screen in CAR T cells co-cultured with GSCs. A, Overview of screen design. CAR T-cells were transduced with a whole-genome CRISPR-Cas9 library and co-cultured with GSCs, followed by a GSC rechallenge after 48 hours. At the conclusion of the screen (24 hours after the rechallenge), CAR T-cells were sorted for PD1 positivity and PD1⁺ or PD1⁻ CAR T-cells were sequenced separately to identify enriched and depleted guides. B, Screen results in two replicates of independent donors with genes ordered alphabetically on the x-axis. The MAGECK β-value for each gene comparing PD1⁻ vs. PD1⁺ is plotted on the y-axis. Genes enriched in PD1⁻ cells at a β-value>1 are in blue or red and genes with a β-value of <−1 (enriched in PD1⁺ cells) are in green or purple. C, Plot of hits from (b) to exclude genes that are depleted following co-culture of CAR T-cells with GSCs (β-value<−1 on the y-axis) or in monoculture (β-value<−1 on the x-axis). Genes in blue or red are not depleted in either condition. D, Venn diagram illustrating common hits for depleted genes in two distinct T cell donors. E, Ingenuity Pathway Analysis of master regulators (top 5 based on β-values) of 220 overlapping genes in two T cell donors. F, Common hits ranked by β-value in a combined model for PD1⁻ vs. PD1⁺ CAR T-cells. Labeled hits were selected for validation.

FIG. 2A-B. CRISPR screening on CAR T cells. A, ClueGO enrichment of GO BP and Reactome pathways in intersected screen hits from both CAR T cell donors. B, Log 2 fold change of normalized counts for each sgRNA targeting TLE4, IKZF2, TMEM184B or EIF5A in the CRISPR-Cas9 screen comparing PD1⁻ to PD1⁺ CAR T cells.

FIG. 3A-J. Targets on CAR T cells improves effector potency and alter transcriptional profiles. A, Killing of CAR T cells with TLE4-, IKZF2-, TMEM184B- or EIF5A-KO against co-cultured GSCs (E:T=1:40, 48 hours). B, Expansion of CAR T cells with different knockouts in co-culture with GSCs (E:T=1:40, 48 hours). C, CAR T cells with targeted KOs of specific genes were co-cultured with PBT030-2 cells (E:T=1:4) for 48 hours, and re-challenged with tumor cells against (E:T=1:8) for 24 hours, and then analyzed for the expression of exhaustion markers. (A,B,C) *p<0.05, **p<0.01, ***p<0.001 compared to CAR T cells transduced with non-targeting sgRNA (black) using unpaired Student's t tests. D and E, Unsupervised clustering of ssGSEA scores comparing TLE4-KO (C) or IKZF2-KO (D) vs. sgCONT CAR T cells for the signatures of selected T cell populations (left) or immune and functional pathways (right). F, Left: Boxplot of genes involved in apoptotic signaling from RNA-sequencing data in sgCONT (blue) vs. sgTLE4 (red). Right: Reactome network of genes downregulated following TLE4 knockout that are involved in apoptotic signaling. G, Left: Boxplot of genes involved in AP1 signaling from RNA-sequencing data in control (blue) vs. TLE4KO (red) cells. Right: Reactome network of genes upregulated with TLE4 knockout that are linked to FOS. Increasing node size and fill hue are proportional to node degree. H, Histogram of log 2 fold change of gene expression (comparing TLE4KO vs. control) for 250 genes previously shown to be upregulated with JUN overexpression. I, Left: Boxplot of genes involved in cytokine receptor signaling from RNA-sequencing data in control (blue) vs. IKZF2KO (red) cells. Right: Reactome network of genes upregulated with IKZF2-KO that are linked to a gene in the cytokine receptor signaling pathway (labeled in red). Increasing node size and fill hue are proportional to node degree. J, Left: Boxplot of genes in the NFAT pathways from RNA-sequencing data in control (blue) vs. IKZF2KO (red) cells. Right: Reactome network of genes upregulated with IKZF2-KO that are linked to upregulated genes in the NFAT pathway (labeled in red).

FIG. 4A-J. Effect of targeted knockouts in CAR T cells. A, IL13Ra2-CAR T cells with targeted KOs of specific genes were co-cultured with PBT030-2 cells (E:T=1:4) for 48 hours, and re-challenged with tumor cells against (E:T=1:8) for 24 hours, and then analyzed for the expression of exhaustion markers. B, IL13Ra2-CAR T cells with targeted KOs of specific genes were co-cultured with PBT030-2 cells (E:T=1:4) for 24 hours and analyzed for early activation markers. C and D, IL13Ra2-CAR T cells with targeted KOs of specific genes were analyzed for CAR expression before GSC stimulation (C), or 3 days after PBT030-2 GSC stimulation (D). E and F, Killing (E) and expansion (F) of HER2-CAR T cells with targeted KOs against co-cultured GSCs (E:T=1:40, 48 hours). (A-F) ns: not significant (p>0.05), *p<0.05, **p<0.01, ***p<0.001 compared to CAR T cells transduced with non-targeting sgRNA (sgCONT, black) using unpaired Student's t tests. G and H, RNA-sequencing of CAR T-cells following TLE4 (G) or IKZF2 (H) knockout (monoculture, 13 days post bead stimulation) plotted as −log 10 FDR (y-axis) vs. log 2 fold change of TLE4 knockout vs. control (x-axis). Blue or red points are genes with <−1.5- or >1.5-fold change, respectively at an FDR of <0.05. I and J, Gene set enrichment analysis for pathways depleted (left, blue) or enriched (right, red) in monoculture CAR T cells following TLE4 (I) or IKZF2 (J) knockout.

FIG. 5A-F. Effect of targeting EIF5A and TMEM184B on CAR T cells. A-B, RNA-sequencing of CAR T-cells following EIF5A (A) or TMEM184B (B) knockout plotted as −log 10 FDR (y-axis) vs. log 2 fold change of TMEM184B knockout vs. control (x-axis). Blue or red points are genes with <−1.5- or >1.5-fold change, respectively at an FDR of <0.05. C-D, Unsupervised clustering of ssGSEA scores comparing EIF5A-KO (C) or TMEM184B-KO (D) vs. control CAR-T cells for selected T cell pathways. E and F, GSEA for pathways depleted (left, blue) or enriched (right, red) following EIF5A (E) or TMEM184B (F) KO.

FIG. 6A-H. The effect of TLE4KO on CAR T cell subpopulations. A, UMAP projection of single cell RNA sequencing of control and TLE4KO CAR T cells both before and after stimulation with GSCs. Cluster assignments for the overall population are shown. B, Cluster composition of unstimulated vs. unstimulated control or TLE4KO cell populations. C, Population distribution of control and TLE4KO CAR T cells before and after stimulation. D, Characterization of clusters based upon cell proliferation. Top: Violin plot of MKI67 expression. Middle: Dot plot of CD4 vs. CD8A expression wherein larger dots indicate a higher proportion of cells with expression and red vs. blue fill indicates higher expression. Heatmap: Scaled expression of T cell markers including costimulatory, activation, naive, exhaustion and regulatory T cell markers as well as AP1 signaling. Bottom: Proportion of cells in each cluster under stimulated vs. unstimulated conditions in control (blue) or TLE4KO (red) populations. Positive values indicate increase in cluster occupancy following stimulation. E-H, Expression of CCL3 (E), TNFRSF4 (F), IFNG (G) and BCAT1 (H) in control or TLE4KO CAR T-cells superimposed on the UMAP projection.

FIG. 7A-J. Single cell transcriptome analysis of TLE4-KO CAR T cells following tumor challenge. A, Left: Proportion of unstimulated (blue) vs. stimulated (red) cells in each cluster, ordered from low to high frequency of stimulated cells. Right: Proportion of sgTLE4 (orange) vs. sgCONT (green) cells in each cluster ordered from low to 4 high frequency of sgTLE4 cells. B, Expression of CD8A (top, green) or CD4 (bottom, red) across clusters. C, Expression of T cell naïve/memory or effector markers across clusters. D, Heatmap of scaled gene expression for the top 10 gene markers of each cluster conserved across all populations (unstimulated and stimulated populations of sgCONT and sgTLE4). Markers were upregulated, significantly differentially expressed genes by Wilcoxon Rank Sum test in one cluster vs. all others across each individual population with a log 2 fold change threshold and minimum percent expression threshold of 0.25. E, expression of FOS and JUN in sgCONT vs. sgTLE4 populations. F, Dot plot of FOS or JUN expression across clusters. Larger dots indicate a higher proportion of expressing cells. Darker color indicates high average expression. G-1, Gene expression in clusters 0, 1 and 10 in sgTLE4 (y-axis) vs. sgCONT (x-axis). Genes in blue are up- or down-regulated at a log 2 fold change>0.4. J, Gene expression changes following stimulation in sgTLE4 (y-axis) vs. sgCONT (x-axis).

FIG. 8A-H. IKZF2 regulates CAR T cell subpopulations. A, UMAP projection of single cell RNA sequencing of control and IKZF2KO CAR T-cells both before and after stimulation with GSCs. Top: Cluster assignments for the overall population. B, Cluster composition of unstimulated vs. unstimulated control or IKZF2KO cell populations. C, Population distribution of control and IKZF2KO CAR T cells before and after stimulation. D, Characterization of clusters based upon cell proliferation. Top: Violin plot of MKI67 expression. Middle: Dot plot of CD4 vs. CD8A expression wherein larger dots indicate a higher proportion of cells with expression and red vs. blue fill indicates higher expression. Heatmap: Scaled expression of T cell markers including costimulatory, activation, naive, exhaustion and regulatory T cell markers as well as AP1 signaling. Bottom: Proportion of cells in each cluster under stimulated vs. unstimulated conditions in sgCONT (blue) or sgIKZF2 (red) populations. Positive values indicate increase in cluster occupancy following stimulation. E, Expression of CXCL10 and CCND1 across clusters (violin plot). F, Expression of top upregulated genes in bulk RNA-seq for sgIKZF2 vs. sgCONT across single cell clusters. G and H, Expression of IFNG(G) and CCL3(H) in sgCONT or sgIKZF2 CAR T-cells superimposed on the UMAP projection.

FIG. 9A-H. Single cell transcriptome analysis of IKZF2-KO CAR T cells following tumor challenge. A, Left: Proportion of unstimulated (blue) vs. stimulated (red) cells in each cluster, ordered from low to high frequency of stimulated cells. Right: Proportion of sgIKZF2 (orange) vs. sgCONT (green) cells in each cluster ordered from low to high frequency of sgIKZF2 cells. B, Expression of CD8A (top, green) or CD4 (bottom, red) across clusters. C, Expression of T cell naive/memory or effector markers across clusters. D, Heatmap of scaled gene expression for the top 10 gene markers of each cluster conserved across all populations (unstimulated and stimulated populations of sgCONT and sgIKZF2). Markers were upregulated, significantly differentially expressed genes by Wilcoxon Rank Sum test in one cluster vs. all others across each individual population with a log 2 fold change threshold and minimum percent expression threshold of 0.25. E and F, ChEA enrichment (E) and pathway analysis (f) of cluster 10 genes that are upregulated at log fold change>0.4 in sgIKZF2 vs. sgCONT. Transcription factors in red are members of the AP1 pathway. G, Gene expression in cluster 10 in sgIKZF2 (y-axis) vs. sgCONT (x-axis). Genes in blue are up- or down-regulated at a log 2 fold change>0.4. H, Gene expression changes following stimulation in sgIKZF2 (y-axis) vs. sgCONT (x-axis).

FIG. 10A-F. CRISPR-Cas9 screen in GSCs co-cultured with CAR T-cells. A, Overview of screen design. GSCs were transduced with a whole-genome CRISPR-Cas9 library and subjected to two rounds of CAR T cell killing (total E:T=1:1). GSCs were then extracted, libraries were prepared, and sequenced to identify enriched and depleted guides. B, Results of the screen in each GSC model. Genes are ordered alphabetically on the x-axis and by MAGECK β score on the y-axis comparing co-culture vs. untreated GSCs. Genes in purple or green are enriched at β>1 (sgRNAs targeting genes that impair GSC killing by CAR T-cells) and those in red or blue are depleted at β<−1 (sgRNAs targeting gene that promote GSC killing by CAR T-cells). C, Plot of depleted genes for each model ordered alphabetically on the x-axis by MAGECK β score on the y-axis comparing untreated day ** vs. day 0. Points in grey are depleted at β<−1 (sgRNAs targeting the gene impair GSC survival). The remaining points in red or blue indicate genes for which knockout do not effect GSC survival. D, Venn diagram illustrating common hits for depleted genes in two models. E, ClueGO plot of GO and Reactome pathways enriched in the union of hits for both models. F, Log 2 fold change of normalized counts for each sgRNA targeting common CRISPR screen hits comparing co-culture to day 0. GSC: glioblastoma stem cell.

FIG. 11A-F. Effect of RELA and NPLOC4 depletion on GSCs and GSC-stimulated CAR T cells. A, GSC viability following knockdown of RELA with one of two independent sgRNAs targeting RELA (top, dark blue and light blue) or NPLOC4 (bottom, orange and red) compared to non-targeting control (black). The controls are the same within each model. B, Expression of IL13Ra2 in GSCs following knockout of NPLOC4 or RELA vs. control. C, Expression of PDL1 in GSCs deleted for NPLOC4 or RELA following co-culture with CAR T-cells (E:T; days). D, Expression of T-cell activation markers CD69, CD137 (24 hours after co-culture with GSCs) and exhaustion markers PD-1, LAG-3 or TIM-3 (72 hours after initial co-culture and 24 hours after rechallenge with GSCs) in CAR T cells against GSCs deleted for NPLOC4, RELA or non-targeting control. E and F, Gene set enrichment plots for upregulated or downregulated immune-related pathways following knockout of RELA (E) or NPLOC4 (F). GSC: glioblastoma stem cell

FIG. 12A-H. RELA or NPLOC4 disruption improves CAR T cell killing of GSCs. A, CAR T cell killing of GSCs (E:T=1:40, 48 hours) with CRISPR-mediated knockout of RELA or NPLOC4. B, CAR T cell expansion in co-culture with GSCs (E:T=1:40, 48 hours) with CRISPR-mediated knockout of RELA or NPLOC4. (a, b) *p<0.05, **p<0.01, ***p<0.001 compared to GSCs transduced with non-targeting sgRNA (black) using unpaired Student's t tests. C, RNA-sequencing of GSCs following RELA knockout plotted as −log 10 FDR (y-axis) vs. log 2 fold change of RELA knockout vs. control (x-axis). Blue or red points are genes with <−1.5- or >1.5-fold change, respectively at an FDR of <0.05. D, Reactome network of genes downregulated following RELA knockout. Only genes linked in the Reactome database to at least one other gene are shown. Node size and color saturation are proportional to node degree. Activating interactions are indicated by arrowheads, while dotted lines indicate predicted interactions. E, Pathway enrichment of genes in the Reactome network of downregulated genes in (c). F, RNA-sequencing of GSCs following NPLOC4 knockout plotted as −log 10 FDR (y-axis) vs. log 2 fold change of NPLOC4 knockout vs. control (x-axis). Blue or red points are genes with <−1.5- or >1.5-fold change, respectively at an FDR of <0.05. G, Reactome network of genes downregulated following NPLOC4 knockout. H, Pathway enrichment of genes in the Reactome network of downregulated genes in (F).

FIG. 13A-C. The role of NPLOC4 in GSCs. A, Interactome maps of IP/MS results of NPLOC4-interacting proteins. B and C, mRNA expression of immune stimulatory cytokines (with TPM reads>0.03 in all samples) in two GSC lines: PBT030-2 (B) and PBT036 (C), color bar indicates relative expression richness comparing each gene between one control sgRNA (sgCONT) and two NPLOC4 knockout (sgNPLOC4-2 and sgNPLOC4-3) groups.

FIG. 14A-J. Functional and clinical relevance of targets on GSCs and CAR T cells. A and B, Kaplan-Meier survival curves comparing mouse survival for RELA (A) or NPLOC4 (B) knockout with non-targeting controls. Tumors were established by orthotopically implanting 2×10⁵ PBT030-2 GSCs, and treated after 8 days with 5×10⁴ CAR T cells. P-values were shown comparing each group with “sgCONT+ CAR” group using Log-rank test. C, Left: Correlation of RELA expression with immune and T cell signatures in TCGA GBM RNA-seq data. Right: Scatter plot of lymphocyte infiltration signature score vs. RELA expression by tumor from TCGA GBM RNA-seq data. D, Left: Correlation of NPLOC4 expression with immune and T cell signatures in TCGA GBM RNA-seq data. Right: Scatter plot of NPLOC4 expression vs. wound healing signature score by tumor from TCGA GBM RNA-seq data. (C, D) p-values were calculated as Pearson's correlation coefficients. E and F, Kaplan Meier curves demonstrating prolonged survival in an intracranial xenograft model of GBM treated with TLE4KO (C) or IKZF2KO (D) CAR T-cells (blue) compared to non-targeting control (black). Tumors were established by orthotopically implanting 2×10⁵ PBT030-2 GSCs, and treated after 8 days with 2×10⁴ CAR T cells. P-values were shown comparing each group with the “CAR sgCONT” group using Log-rank test. FDR: False discovery rate. G, Fold change after stimulation of genes significantly upregulated (FDR<0.05, Log 2 fold change>1) following IKZF2 knockout after tumor stimulation, in an independent dataset of clinical CAR T cells products from patients with CLL. H, Fold change after stimulation of genes enriched in cluster 10 as shown in FIG. 5. I and J, Fold change after stimulation of genes significantly upregulated (FDR<0.05, Log 2 fold change>1) following IKZF2 (I) or TLE4 (J) knockout. (G-J) CAR T cells were stratified by response of the patient from which they were derived—complete responders or non-responders—to CAR therapy and the log 2 fold change of stimulated vs. mock-stimulated gene expression was plotted, p-values were calculated by unpaired Student's t tests.

FIG. 15A-B. Bioluminescent imaging of tumor-bearing mice after CAR treatment. A, Orthotopic tumors established by PBT030-2 GSCs with targeted knockouts or control sgRNA (sgCONT), as shown in FIG. 14A and B, received CAR treatment (CAR) or no treatment (no CAR). B, Orthotopic tumors established by wild-type PBT030-2 GSCs were treated by CAR T cells with targeted knockouts or control sgRNA (sgCONT), as shown in FIGS. 7E and 7F. (A, B) CAR T cells were injected 8 days after tumor inoculation, “X” indicates mice that were euthanized before the designated imaging time point.

FIG. 16A-D. High RELA and NPLOC4 expression correlates with suppression of antitumor T cells in GBMs. A and B, Immune suppressive signatures, including tumor immune dysfunction and exclusion (TIDE, ref X), and immune checkpoint blockade resistance (ref X), in GSC models of high and low expression of RELA (A) or NPLOC4 (B). p-values were calculated using the Mann-Whitney test. C and D, Correlation analysis of GBM TCGA dataset between the infiltration of CD4+ memory/CD8+ T cells and the expression of RELA (C) or NPLOC4 (D), analyses and statistics were performed through http://timer.cistrome.org FIG. 17A-B. Effect of TMEM184B- and EIF5A-KO on CAR T cell in vivo function. Kaplan Meier curves demonstrating prolonged survival in an intracranial xenograft model of PBT003-2 GBM treated with sgTMEM184B (A) or EIF5A (B) CAR T cells compared to non-targeting control (black). P-values were shown comparing each group with the “CAR sgCONT” group using Log-rank test.

FIG. 18A-E. High IKZF2 expression correlates with CAR T cell dysfunction. A, UMAP projection of single cell RNA sequencing data from 24 CD19-CAR T cell products (GSE151511). B, Expression of IKZF2 as superimposed on the UMAP projection. C and D, Expression of IKZF2 and other Treg-associated genes (CTLA4, FOXP3, IL2RA) across single cell clusters. E, Proportions of cells coming from the CAR T cell products from patients with complete response (CR) or progress disease (PD) across single cell clusters. Proportions were normalized to total cells within each cluster. Dotted line indicates proportions of cells from CR and PD patients combining all cells analyzed.

FIG. 19A-F. Exhausted CAR T cells showed high TLE4 activity. A, UMAP projection of single cell RNA sequencing data from 24 CD19-CAR T cell products in a previously published study (GSE151511). B, Expression of TOX, TOX2 and TLE4-repressed genes across different clusters. C, Heatmap on scaled expression of T cell markers including costimulatory, activation, naive, exhaustion and regulatory T cell markers. D and E, Expression of TLE4-repressed genes as superimposed on the UMAP projection (D) and across different clusters (E). F, TLE4 expression of 4 CD19-CAR T cell products at different times after CAR engineering.

FIG. 20A-B. sgRNA counts in samples for CRISPR screening. Counts of all sgRNAs in the screening library in Day 0 samples of CAR T cells from two independent donors (A) and two independent GSC samples (B).

FIG. 21. CRISPR screening strategy to potentiate CAR T cell therapy. Screening on both GSCs and CAR T cells identified targets that increase GSC sensitivity to CAR killing or augment CAR T cell effector activity. Targeted genetic deletions on GSCs or CAR T cells modified critical pathways of immune reactivity and T cell activation, enhancing cytotoxic effect against GSCs and in vivo antitumor effect.

### DETAILED DESCRIPTION

GSCs represent a potentially important cellular target in GBM, as they have been linked to therapeutic resistance, invasion into normal brain, promotion of angiogenesis, and immune modulation (24,25). We hypothesized that systematic interrogation of molecular regulation of CAR T cell efficacy against GBM could be optimized by screening both CAR T cells and GBM cells, thereby informing the interplay between a cell-based therapy and its target population. Here, we developed a robust method for performing whole-genome CRISPR-knockout screens in both GBM cells and human CAR T cells. Using our well-established CAR T cell platform targeting the tumor-associated surface marker interleukin-13 receptor α2 (IL13Rα2) (7,8,26), we identified novel CAR T cell- and tumor-intrinsic targets that substantially improved CAR T cell cytotoxicity against GSCs both in vitro and in vivo. Targeted genetic modification of identified hits in CAR T cells potentiated their long-term activation, cytolytic activity, and in vivo antitumor function against GSCs, demonstrating that CRISPR screen on CAR T cells leads to the discovery of key targets for augmenting CAR T cell therapeutic potency. In parallel, knockout of identified targets in GSCs sensitized them to CAR-mediated killing both in vitro and in vivo, revealing potential avenues for combinatorial inhibitor treatment to augment CAR T cell efficacy. Our findings represent a feasible and highly effective approach to discovering key targets that mediate effective tumor eradication using CAR T cells.

### Examples

The invention is further described in the following examples, which do not limit the scope of the invention described in the claims.

### Materials and Methods

### Lentiviral Transduction on GSCs

GSCs were acquired from patient specimens at City of Hope under protocols approved by the IRB, and maintained as tumorspheres in GSC media as previously described (4,91). GSC lines used in this study to test CAR T cell function are IDH1/2-wildtype. The sgRNA library and single-targeted sgRNA lentiviral plasmids (containing a puromycin-resistance gene) for GSC transduction were purchased from Addgene (#73179 and #52961, respectively). Lentiviral particles were generated as previously described (92). For lentiviral transduction, GSC tumorspheres were dissociated into single cells using Accutase (Innovative Cell Technologies), resuspended in GSC media and lentivirus was added at a 1:50 v/v ratio. GSCs were then washed once after 12 hours, resuspended in fresh GSC media and cultured for 3 days. To ensure that only transduced cells were expanded for further assays, GSCs were selected by puromycin (Thermo Fisher Scientific) for 7 continuous days, with a 1:10000 v/v ratio into GSC media.

### Lentiviral Transduction on Primary Human T Cells

Naïve and memory T cells were isolated from healthy donors at City of Hope under protocols approved by the IRB (26,30). The constructs of IL13Ra2-targeted and HER2-targeted CARs were described in previous studies (8,26,93). Procedures of CAR-only transduction on primary human T cells were previously described (44). The sgRNA library and single-targeted sgRNA lentiviral plasmids for T cell transduction were purchased from Addgene (#73179 and #52961, respectively). All sgRNA plasmids contain a puromycin-resistance gene. Dual transduction of CAR and sgRNA were performed using modification of previously reported procedures (21). In brief, primary T cells were stimulated with Dynabeads Human T expander CD3/CD28 (Invitrogen) (T cells: beads=1:2) for 24 hours and transduced with sgRNA lentivirus (1:250 v/v ratio). Cells were washed after 6 hours and then transduced with CAR lentivirus (multiplicity of infection [MOI]=0.5). 4 days after CAR transduction, CD3/CD28 beads were removed and cells were resuspended in Lonza electroporation buffer P3 (Lonza, #V4XP-3032) (2×10⁸ cells/mL). Cas9 protein (MacroLab, Berkeley, 40 mM stock) was then added to the cell suspension (1:10 v/v ratio) and electroporation was performed using a 4D-Nucleofactor™ Core Unit (Lonza, #AAF-1002B). Cells were recovered in pre-warmed X-VIVO 15 media (Lonza) for 30 min before proceeding to ex vivo expansion. All T cell transduction and ex vivo expansion experiments were performed in X-VIVO 15 containing 10% FBS, 50 U/ml recombinant human IL-2 (rhIL-2), and 0.5 ng/ml rhIL-15, at 6×10⁵ cells/ml. To ensure that only sgRNA-transduced cells were expanded, puromycin (1:10000 v/v ratio) was added to the media 3 days after electroporation, and puromycin selection was performed for 6 continuous days before CAR T cells were used for further assays. CRISPR screening was performed on two independent donors, and other 2 donors are used to generate IL13Ra2-targeted and HER2-targeted CARs, respectively.

### CRISPR Screening on GSCs

GSCs transduced with the CRISPR KO library were dissociated into single cells, and co-cultured with CAR T cells at an effector: target ratio of 1:2 in culture plates pre-coated with matrigel. After 24 hours, the media containing CAR T cells and tumor debris were removed, and same number of CAR T cells were added in fresh media. 24 hours after the second CAR T cell addition, the media were removed and remaining GSCs were washed with PBS and harvested. Genomic DNA was isolated from the remaining GSCs after co-culture with CAR T cells, as well as GSCs harvested before co-culture and GSCs after monoculture for 48 hours.

### CRISPR Screening on CAR T Cells

T cells transduced with CAR and the CRISPR KO library were co-cultured with GSC at an effector: target ratio of 1:4 in culture plates pre-coated with matrigel. After 48 hours, CAR T cells were re-challenged by GSCs doubling the number of the initial co-culture. 24 hours after the rechallenge, the co-culture was harvested and stained with fluorescence-conjugated antibodies against human CD45 (BD Biosciences Cat #340665, RRID:AB_400075), PD1 (BioLegend Cat #329922, RRID:AB_10933429) and IL13 (BioLegend Cat #501914, RRID:AB_2616746). Different subsets were sorted using an Aria SORP (BD Biosciences): total CAR T cells (CD45+, IL13+), PD1⁺ CART cells (CD45+, IL13+, PD1⁺) and PD1− CART cells (CD45+, IL13+, PD1−). Genomic DNA was isolated from the sorted subsets of cells, as well as CAR T cells harvested before co-culture and CAR T cells after monoculture for 72 hours.

### CRISPR-Cas9 Screen Analysis

FASTQ files were trimmed to 20 bp CRISPR guide sequences using BBDuk from the BBMap (https://jgi.doe.gov/data-and-tools/bbtools) (RRID:SCR_016965) toolkit and quality control as performed using FastQC (RRID:SCR_014583, https://www.bioinformatics.babraha-m.ac.uk/projects/fastqc/). FASTQs were aligned to the library and processed into counts using the MAGECK-VISPR ‘count’ function (https://bitbucket.org/liulab/mageck-vispr/src/master/). β-values were calculated using an MLE model generated independently for each comparison. Non-targeting sgRNAs were used to derive a null distribution to determine p-values.

### In Vitro Cytotoxicity and Flow Cytometry Assays

For in vitro cytotoxicity test, CAR T cells were co-cultured with GSCs at an effector: target ratio of 1:40. After 48 hours of co-culture, the numbers of CAR T cells and GSCs were evaluated by flow cytometry. Flow cytometry assays were performed on GSCs, CAR T cells from monoculture or co-culture with procedures described previously (30). For co-culture, anti-CD45 (BD Biosciences Cat #340665, RRID:AB_400075) staining was used to distinguish GSCs with T cells, and CAR T cells were identified by anti-IL13 (BioLegend Cat #501914, RRID:AB_2616746) staining. Other antibodies used for flow cytometry target: PD-L1 (Thermo Fisher Scientific Cat #17-5983-42, RRID:AB_10597586), TIM3 (Thermo Fisher Scientific Cat #17-3109-42, RRID:AB_1963622), LAG3 (Thermo Fisher Scientific Cat #12-2239-41, RRID:AB_2572596), PD1 (BioLegend Cat #329922, RRID:AB_10933429), CD69 (BD Biosciences Cat #340560, RRID:AB_400523), CD137 (BD Biosciences Cat #555956, RRID:AB_396252) and IL13Ra2 (BioLegend Cat #354404, RRID:AB_11218789). All samples were analyzed via a Macsquant Analyzer (Miltenyi Biotec) and processed via FlowJo v10 (RRID:SCR_008520).

### RNA-Sequencinq Analysis

Total mRNA from GSCs or CAR T cells was isolated and purified by RNeasy Mini Kit (Qiagen Inc.) and sequenced with Illumina protocols on a HiSeq 2500 to generate 50-bp reads. Trim Galore (https://www.bioinformatics.babraham.ac.uk/projects/trim_galore/) (RRID:SCR_011847) was used to trim adaptors and remove low quality reads. Reads were quantified against Gencode v29 using Salmon (RRID:SCR_017036, https://combine-lab.github.io/salmon/) with correction for fragment-level GC bias, positional bias and sequence-specific bias. Transcripts were summarized to gene level and processed to transcripts per million (TPM) using the R/Bioconductor (https://www.bioconductor.org/) package DESeq2 (RRID:SCR_000154, https://bioconductor.org/packages/release/bio-c/html/DESeq2.html). Comparisons were performed using contrasts in DESeq2 followed by Benjamini-Hochberg adjustment to correct for false discovery rate.

### Gene Set Enrichment Analysis

ClueGO gene set enrichment plots were generated using the ClueGO plugin (http://apps.cytoscape.org/apps/cluego, RRID:SCR_005748) for GO BP, KEGG or Reactome gene sets and visualized in Cytoscape v3.7.2 (https://cytoscape.org/).

GSEA (RRID:SCR_003199) plots were generated from preranked lists using the mean β value as the ranking metric. Reactome networks were created using the Reactome FI plugin (https://reactome.org/tools/reactome-fiviz) with network version 2018 and visualized in Cytoscape. Networks were clustered using built-in network clustering algorithm, which utilizes spectral partition-based network clustering, and node layout and color were determined by module assignment. GSEA plots from RNA-sequencing data were generated from preranked lists. Weighting metrics for preranked lists were generated using the DESeq2 results from the gene knockdown vs. non-targeting control and applying the formula: −log 10(FDR)*log 2(fold change). ssGSEA scores for specific immune or functional pathways were generated using the ssGSEA function from the R/Bioconductor package GSVA (https://bioconductor.org/packages/release/bioc/html/GSVA.html) (94) (93) (93) and plotted using pheatmap (https://cran.r-project.org/web/packages/pheatmap/). ChEA enrichments were performed using Enrichr (https://amp.pharm.mssm.edu/Enrichr/). Barplots for positive or negative gene set enrichments were performed using Metascape (https://metascape.org/gp/index.html) for significantly up- or down-regulated genes (FDR<0.05 and log 2 fold change>1 or <−1).

### Reactome Networks and KEGG Pathways

Reactome networks were derived from RNA-seq data using the Cytoscape Reactome FI plugin (RRID:SCR_003032). A gene list of upregulated (FDR<0.05 and log 2 fold change>1) or downregulated (FDR<0.05 and log 2 fold change<−1) genes plus the target gene (as knockout by CRISPR-Cas9 would not be detected by RNA-seq) was input into Reactome FI and all genes with at least one edge were included in the network plot. Node color (light to dark) and size (small to large) are proportional to node degree. Pathway enrichment was performed on this network of genes using the Reactome FI enrichment option. Boxplots for genes from selected pathways were generated using RNA-seq TPM data. KEGG pathway visualizations were generated using the R/Bioconductor package pathview (https://www.bioconductor.org/packages/release/bioc/html/pathview.html) from for selected pathways and genes were colored based upon the log 2 fold change knockout vs. control.

### Single Cell RNA-Sequencinq Analysis

Single cell RNA-sequencing files were processed using the Cell Ranger workflow (https://support.10xgenomics.com/single-cell-gene-expression/software/overview/welcome). FASTQ files were generated using the Cell Ranger ‘mkfastq’ command with default parameters. FASTQs were aligned to the hg19 genome build using the ‘count’ function and aggregated using the default Cell Ranger ‘aggr’ parameters with normalization performed by subsampling wells to equalize read depth across cells. Downstream analyses were performed using the R/Bioconductor package Seurat (https://satijalab.org/seurat/) (95)(94)(95). Specifically, datasets of stimulated and unstimulated cells in knockout or control populations were merged using the “FindintegrationAnchors” Seurat function. Clustering was performed using UMAP using PCA for dimensional reduction and a resolution of 0.6 from 1 to 20 dimensions. Dead cell clusters were determined by high expression of mitochondrial genes and removed. Samples were then reclustered. Clusters with similar CD4 or CD8, Ki67 and marker expression, determined using the “FindAllMarkers” function that were proximal on the UMAP projection were merged. All plots for gene expression were generated using normalized data from the default parameters of the “NormalizeData” function. Gene expression was visualized on the UMAP projection using the “FeaturePlot” function with a maximum cutoff or gene expression determined on a gene-by-gene basis.

### Functional Analysis of CAR T Cells in Orthotopic GBM Models

All mouse experiments were performed using protocols approved by the City of Hope IACUC. Orthotopic GBM models were generated using 6- to 8 week-old NOD/SCID/IL2R^{-/-} (NSG) mice (IMSR Cat #JAX:005557, RRID:IMSR_JAX:005557), as previously described (96). Briefly, ffLuc-transduced GSCs (1×10⁵/mouse) were stereotactically implanted (intracranially) into the right forebrain of NSG mice. Randomization was performed after 8 days of tumor injection based on bioluminescent signal, and mice were then treated intracranially with CAR T cells (2×10⁴ or 5×10⁴/mouse as indicated for each experiment). To ensure statistical power, all treatment groups include ≥6 animals. Mice were monitored by the Department of Comparative Medicine at City of Hope for survival and any symptoms related to tumor progression, with euthanasia applied according to the American Veterinary Medical Association Guidelines. Studies were done in both male and female animals. Investigators were not blinded for randomization and treatment.

### TCGA Data Analysis

Analysis of genes in the TCGA dataset was performed using RNA-sequencing TCGA GBM data. Immune infiltration signatures were previously reported (97). GSEA plots for each gene in the context of TCGA GBM data were generated by using the normalized gene expression as a continuous phenotype.

### CAR T Cell Responder Analysis

Gene sets derived from TLE4 or IKZF2 knockout were analyzed in the context of CAR T cell non-responder vs. responders from a previous report on patients with CLL (27). Genes upregulated in bulk RNA-seq of CAR T cells following knockout of TLE4 or IKZF2 (FDR<0.05 and log 2 fold change>1) were plotted by their fold change expression in stimulated vs. unstimulated CAR T cells for responders or non-responders. Fold change was calculated using DESeq2 for stimulated vs. unstimulated cells independently for each group (non-responder or complete responder). Cluster 10-enriched genes in the TLE4 knockout and control sc-seq data, identified by the “FindAllMarkers” function in Seurat subsetted for overexpressed genes, were plotted similarly. Genes upregulated (>0.4 log 2 fold change of normalized counts) in sc-seq for IKZF2 knockout vs. control in stimulated CAR T cells were plotted similarly.

### Statistical Analysis

CAR T cell functional data (tumor killing, expansion, survival of tumor-bearing mice) were analyzed via GraphPad Prism. Group means±SEM were plotted. Methods of p-value calculations are indicated in figure legends.

### Example 1:Genome-Wide CRISPR Screening of CAR T Cells Identifies Essential Regulators of Effector Activity

The fitness of CAR T cell products correlates with clinical responses (27,28), indicating that key regulators of CAR T cell function can be targeted to potentiate therapeutic efficacy. T cell exhaustion resulting from chronic tumor exposure limits CAR T cell antitumor responses (29). To identify the essential regulators of T cell functional activity in an unbiased manner, we performed genome-wide CRISPR screen adapting our previously developed in vitro tumor rechallenge assay, which differentiates CAR T cell potency in the setting of high tumor burden and reflects in vivo antitumor activity (30,31). IL13Ra2-targeted CAR T cells from two human healthy donors were lentivirally transduced to express the Brunello short-guide RNA (sgRNA) library (32) and the CAR construct, then electroporated with Cas9 protein.

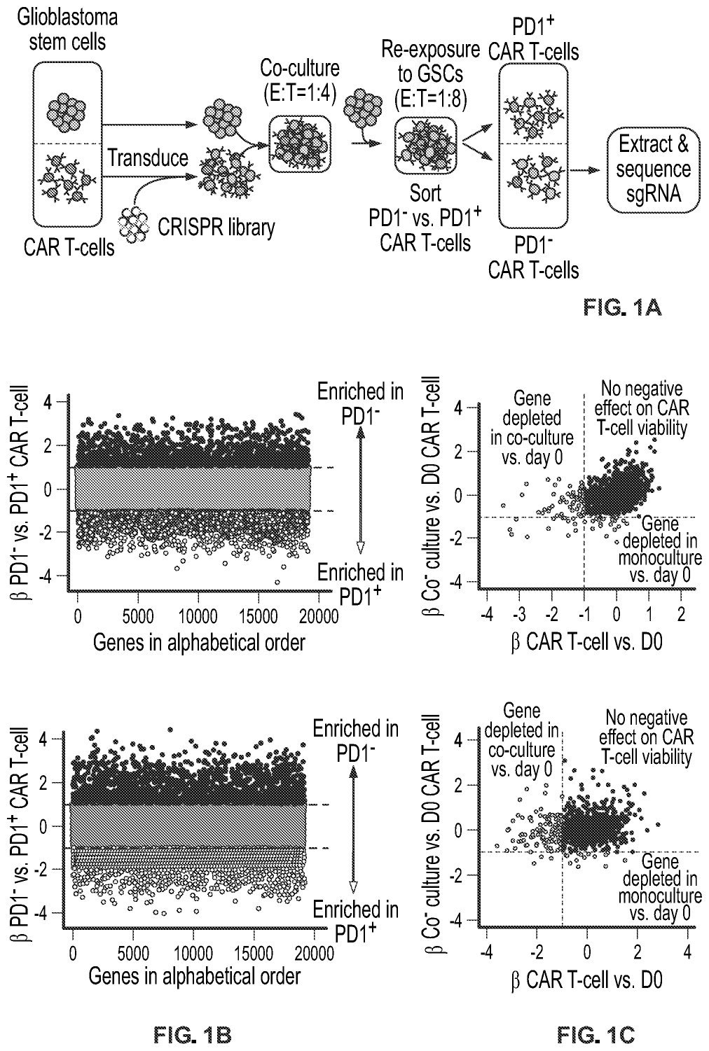

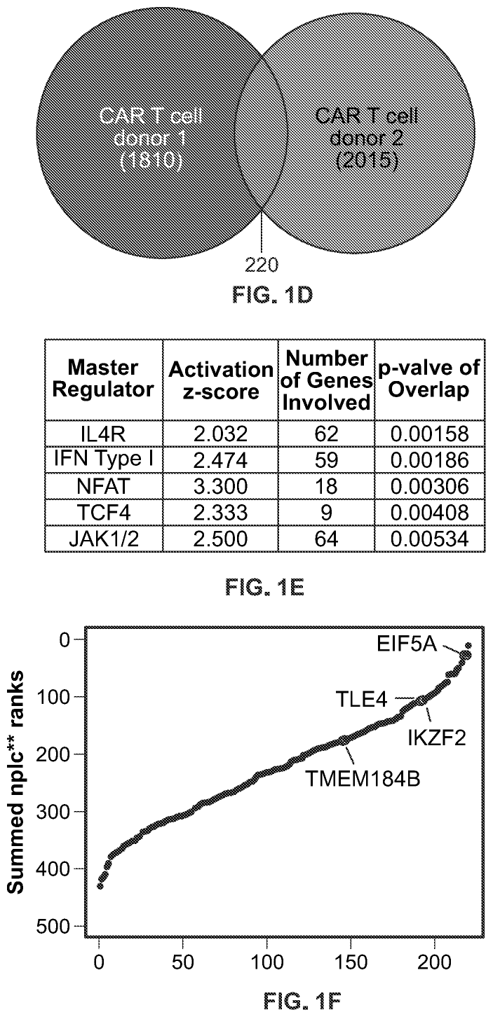

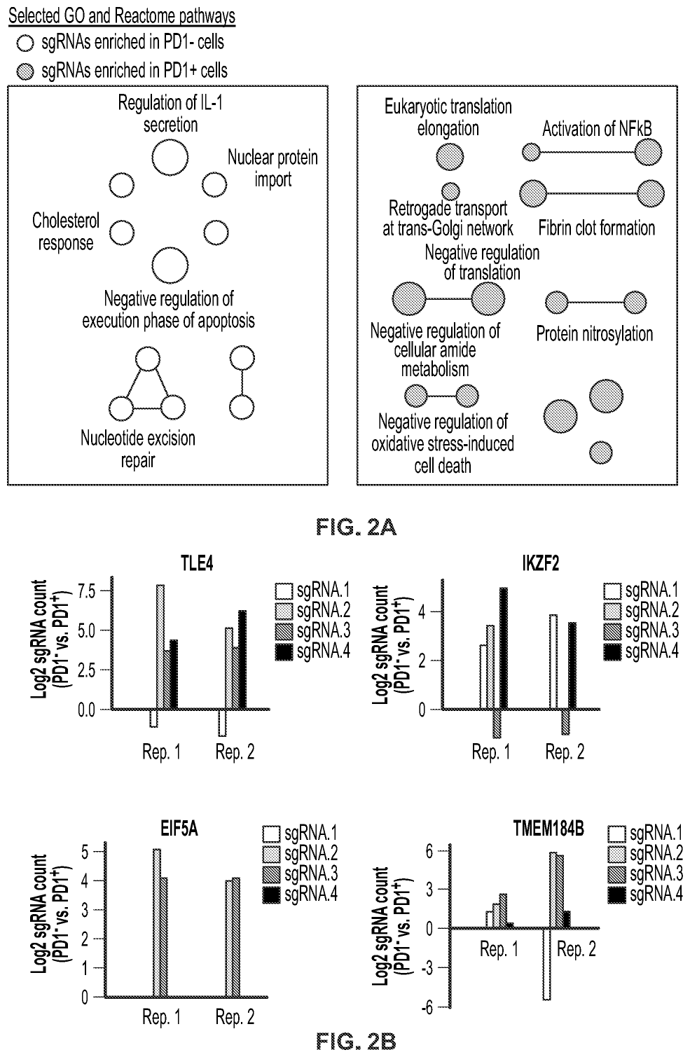

CAR T cells harboring CRISPR-mediated knockouts were recursively exposed to an excess amount of PBT030-2 GSCs (FIG. 1A), an IDH1 wild-type patient-derived GSC line that highly expresses IL13Ra2 (33). After tumor stimulation, CAR T cells were sorted from co-culture and subsetted based on expression of the inhibitory receptor PD-1, which is associated with T cell exhaustion (FIG. 1A). To identify gene knockouts that augment CAR T antitumor activity, we identified sgRNAs enriched in the less exhausted PD1-negative versus PD1-positive CAR T cell compartments (FIG. 1B). To eliminate targets that non-specifically impaired CAR T cell proliferation or viability, we excluded sgRNAs depleted (β-value<−1) in CAR T cells after 72-hour co-culture with GSCs or 72-hour monoculture (FIG. 1C). 220 genes were common hits in both T cell donors (FIG. 1D). Many of these 220 genes are induced by the IL4 receptor (IL4R), which suppresses T cell activity (34), as well as Type I Interferon, NFAT, TCF4, and JAK1/2, which all play complex roles on T cell activation and mediate T cell exhaustion and inhibition under some circumstances (35-38) (FIG. 1E). Additionally, these genes were enriched for pathways that contribute to T cell exhaustion, including nuclear receptor transcription and cholesterol responses (39,40) (FIG. 2A). In contrast, genes preferentially depleted in PD1-positive cells included pathways associated with of T cell activation, including amide metabolism and NF-κB signaling (41,42), as well as negative regulation of oxidative stress-induced cell death (FIG. 1E). Together, this data verifies that our screen identified genes involved in T cell effector activity, providing candidate genes which can be modulated to prevent exhaustion and enhance effector function of CAR T cells.

### Example 2: CRISPR Screening Empowers Discovery of Targets that Enhance CAR T Cell Cytotoxic Potency

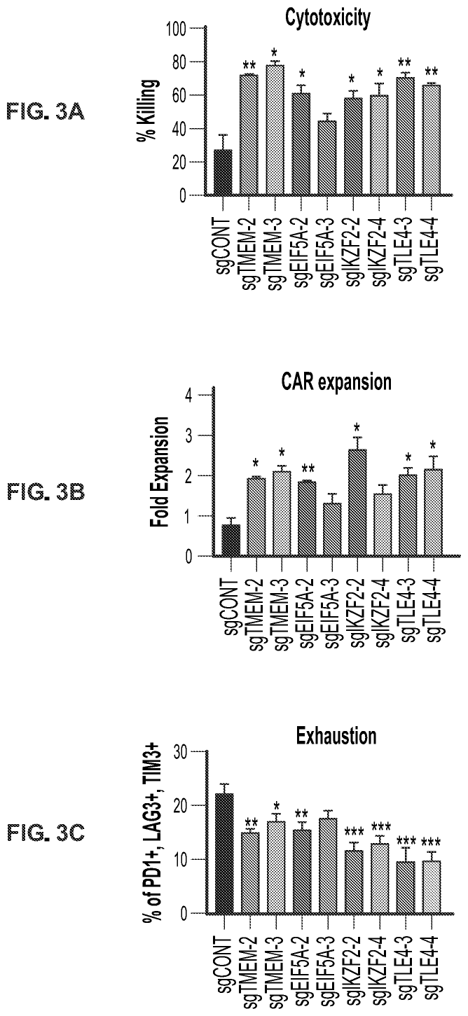

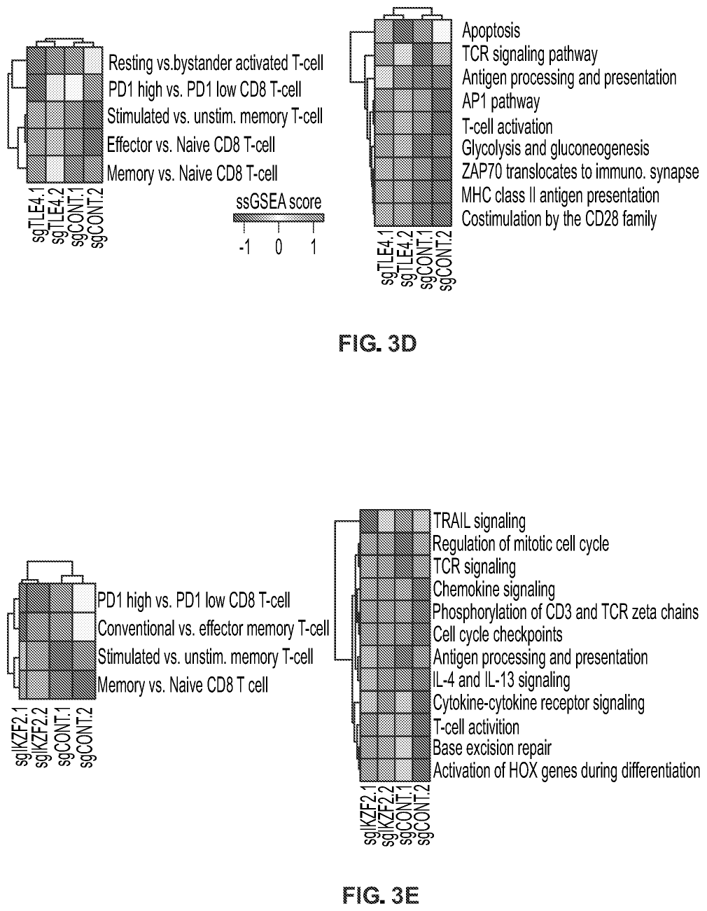

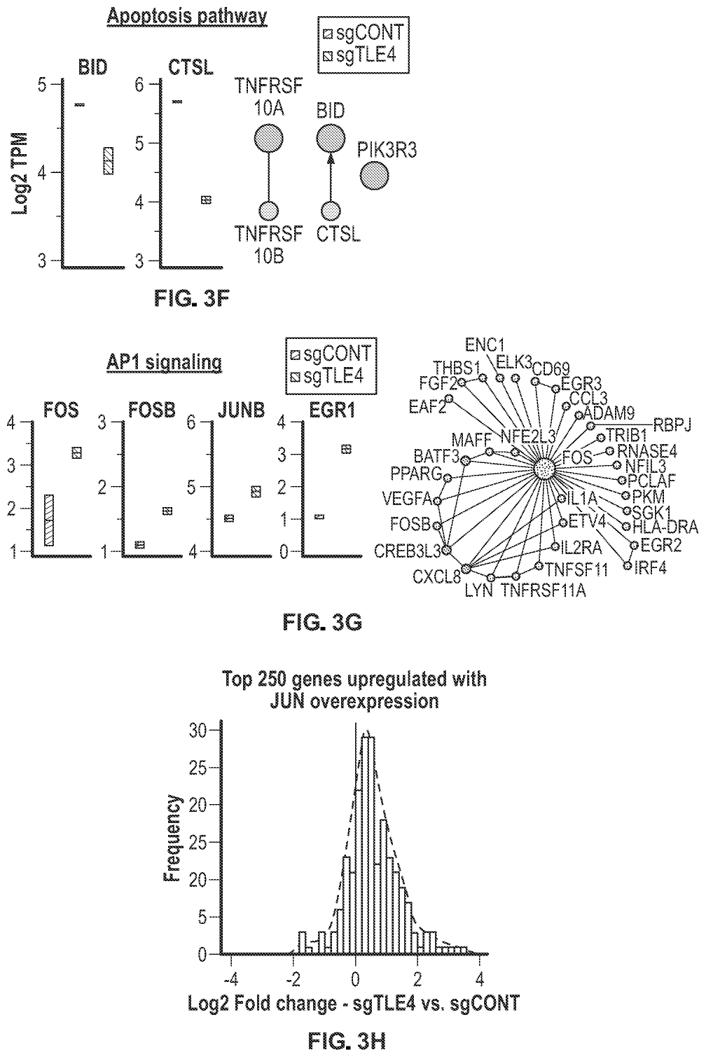

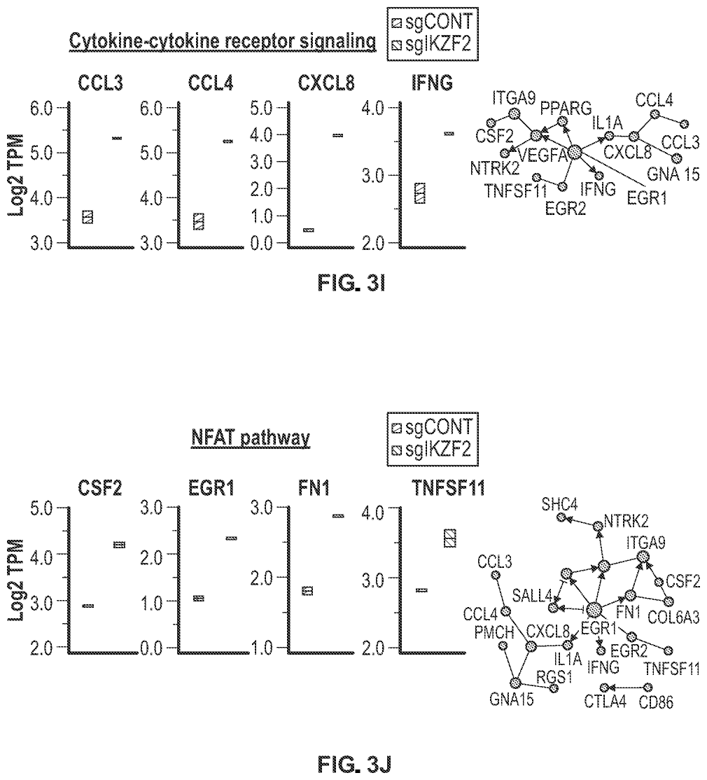

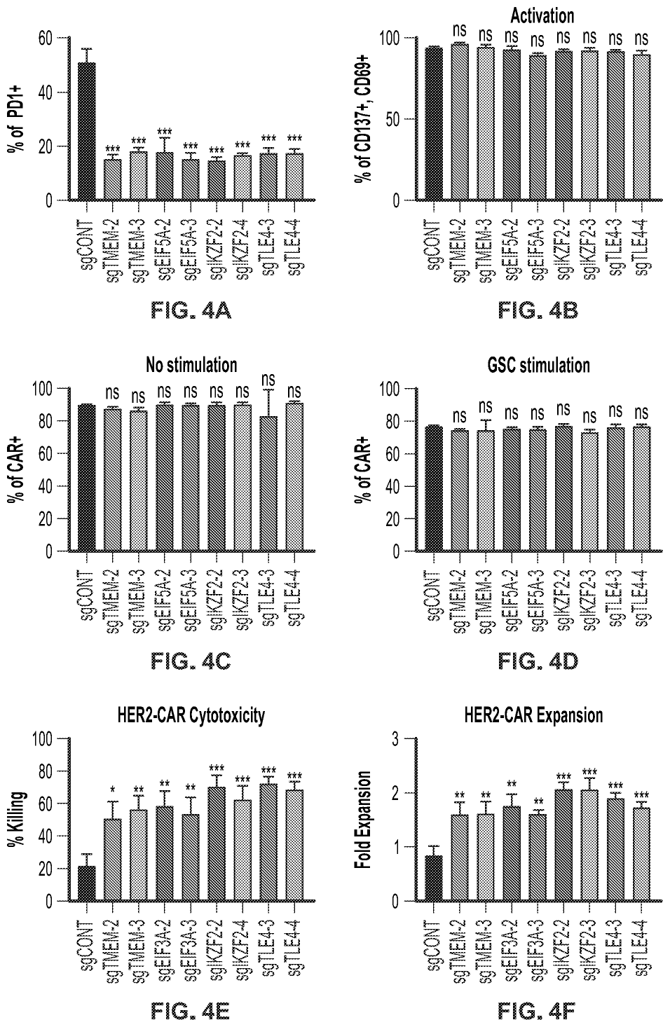

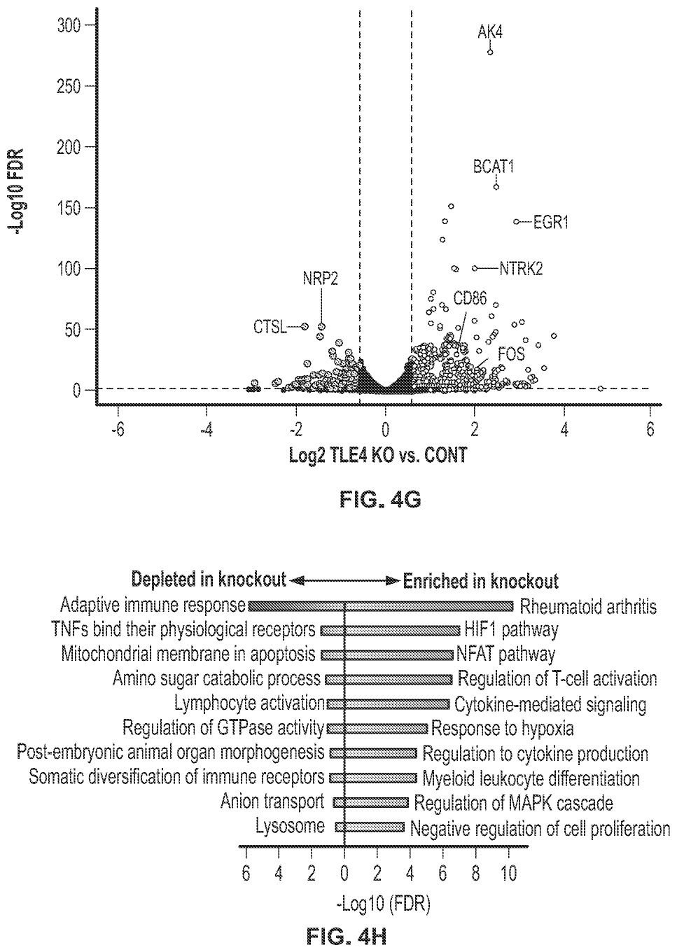

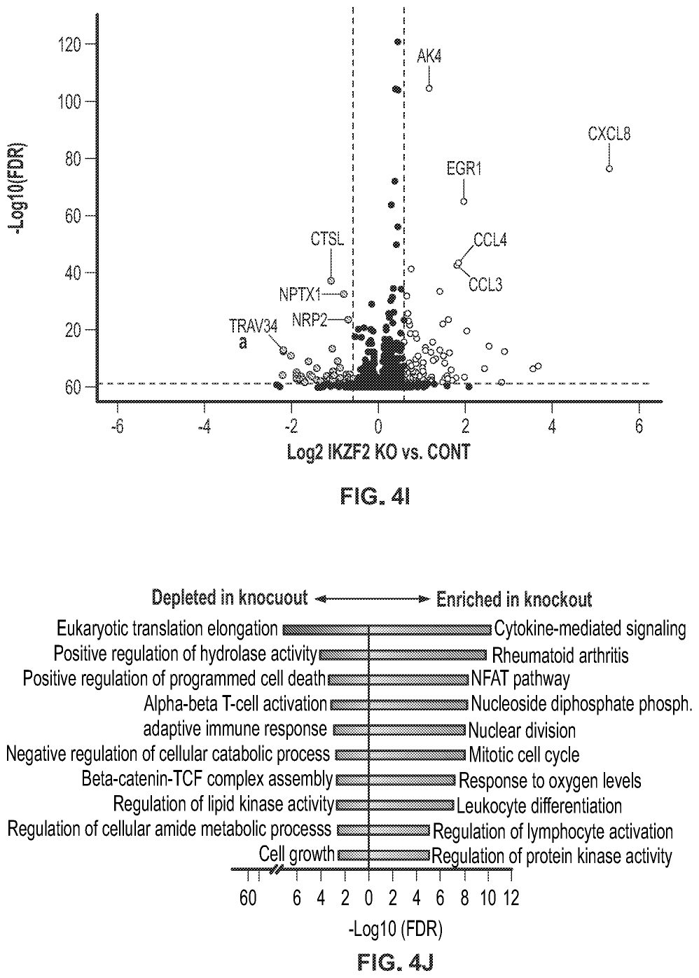

We interrogated the 220 targets enriched in PD1-negative cells common between two T cell donors, focusing on four representative genes identified in the top third of hits, which have not been previously explored for their role in enhancing CAR T cell function. These included the high-ranking hits: Eukaryotic Translation Initiation Factor 5A-1 (EIF5A; Gene ID 1984), transcription factor Transducin Like Enhancer of Split 4 (TLE4; Gene ID 7091), Ikaros Family Zinc Finger Protein 2 (IKZF2; Gene ID 22807), and Transmembrane Protein 184B (TMEM184B; Gene ID 25829) (FIG. 1F). Gene IDs can be located at www.ncbi.nlm.nih.gov. Most sgRNAs targeting these genes (2 out of 4 in both replicates) were enriched in PD1-negative CAR T cells (FIG. 2B). To verify the function of these targets by CRISPR-mediated KO on CAR T cells we leveraged the challenging in vitro killing assay (CAR:Tumor=1:40), confirming that targeting TLE4, IKZF2, TMEM184B, or EIF5A improved in vitro killing potency of CAR T cells against GSCs, as well as the their expansion potential, although sgEIF5A-3 effects were more modest (FIGS. 3A and B). Mechanistically, KO of these genes reduced PD-1 expression on CAR T cells following tumor stimulation (FIG. 4A). We and others have shown that CAR T cell exhaustion is associated with co-expression of PD-1, LAG-3, and TIM-3 (43,44). All four KOs reduced CAR T cell exhaustion; TLE4- and IKZF2-KO most effectively (FIG. 3C). KO of these genes minimally affected initial CAR T cell activation upon tumor cell recognition (FIG. 4B), suggesting that these KOs improved T cell fitness and long-term function instead of initial activation. Targeted KOs did not affect the expression and stability of the CAR in T cells (FIGS. 4C and 4D). As validation, we performed independent studies with a HER2-targeted CAR model that also demonstrated improvements in CAR killing and expansion, suggesting that genetic screens of CAR T cells may yield broadly effective molecular strategies (FIGS. 4E and 4F).

TLE4 is a transcriptional co-repressor of multiple genes encoding inflammatory cytokines (45) and IKZF2 is upregulated in exhausted T cells (37,46,47), supporting potential roles in inhibiting CAR T cell function. To elucidate molecular mechanisms underlying the regulation of CAR T cell activity, we compared the transcriptomes of CAR T cells with individual knockouts against cells transduced with non-targeted sgRNA (sgCONT). TLE4 KO in CAR T cells upregulated critical regulators of T cell activation, including the transcription factor EGR1, which promotes Th1 cell differentiation (48), and the metabolic regulator BCAT, which mediates metabolic fitness in activated T cells (49) (FIG. 4G). IKZF2 KO in CAR T cells upregulated proinflammatory cytokines and pathways, including CXCL8, CCL3, and CCL4 (50-52), as well as EGR1, similar to TLE4 KO (FIG. 4H). We next compared transcriptional profiles of TLE4 or IKZF2 KO CAR T cells to the signatures of known T cell subsets and pathways (35,53,54). TLE4 or IKZF2 KO induced molecular signatures representing activation over memory T cells, together with key T cell activation signaling pathways (TCR signaling, T cell activation, AP-1, and ZAP) (FIGS. 3D and 3E; FIGS. 4I and 4J). T cell activation characteristics in TLE4-KO or IKZF2-KO cells were uncoupled from exhaustion (FIGS. 3D and 3E), suggesting retention of CAR T cell function. TLE4-KO cells downregulated an apoptosis signature (FIGS. 3D and 3F) and upregulated AP-1 signaling, which maintains CAR T cell function (55) (FIG. 3G). In particular, the AP-1 family transcription factor FOS was enriched after TLE4 KO, together with many of its downstream targets (FIG. 3G). As overexpression of the AP-1 family member JUN prevents CAR T cell exhaustion (55), we investigated whether TLE4 KO phenocopied transcriptional changes of JUN overexpression, revealing that genes upregulated with TLE4 KO overlapped with genes with upregulated following JUN overexpression (FIG. 3H). IKZF2 KO upregulated pathways involving interactions between cytokines and their receptors, as well as NFAT signaling, which regulates key molecular signals following T cell activation (56) (FIGS. 31 and 3J). As EGR1 was upregulated after IKZF2 KO, many genes in these pathways were likely downstream targets (FIGS. 31 and 3J).

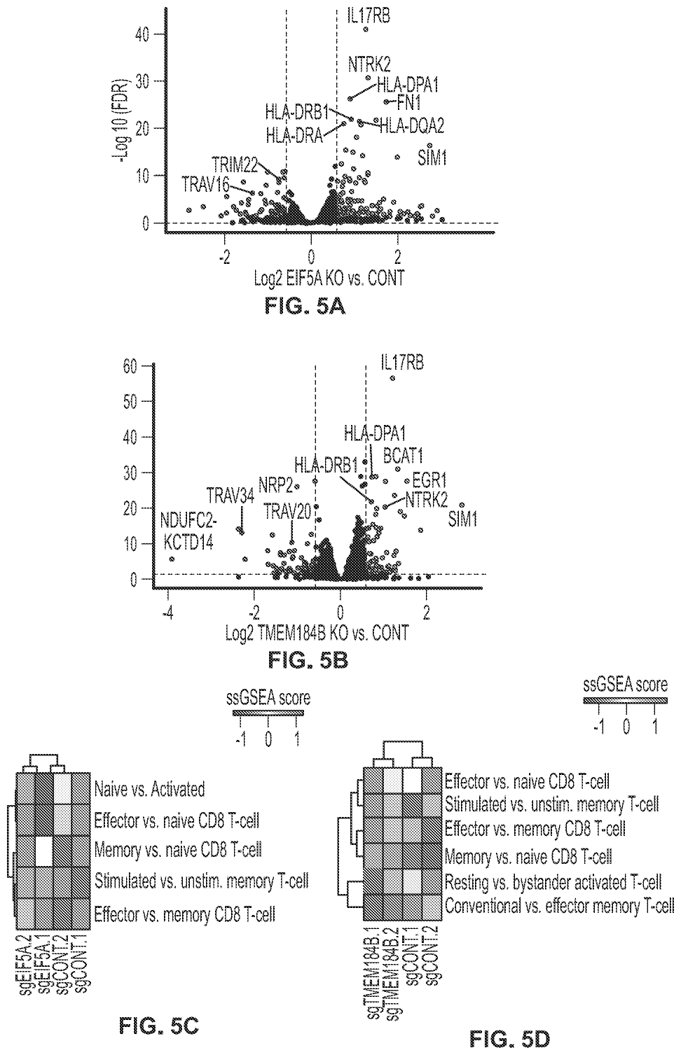

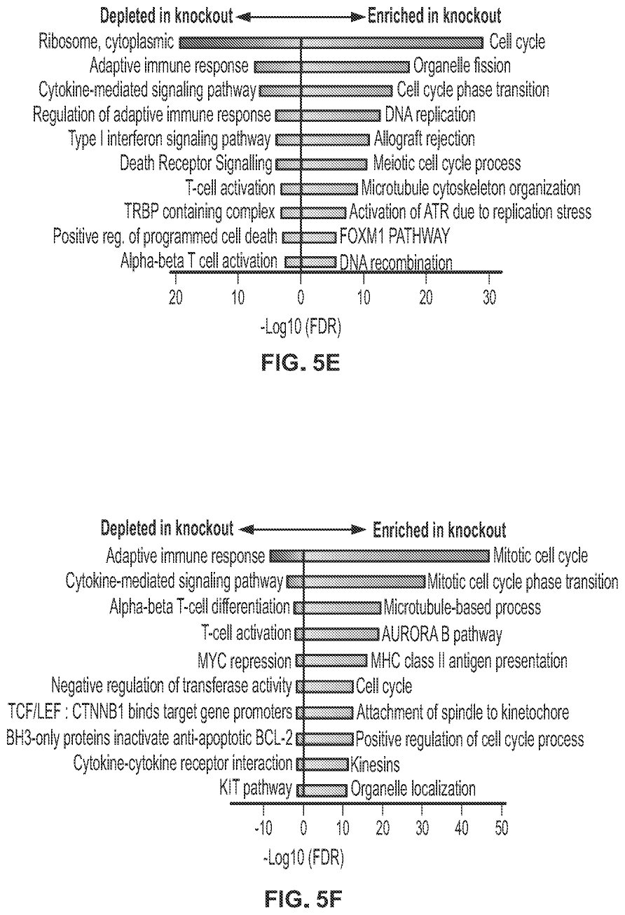

Whole-transcriptome analyses following TMEM184B or EIF5A KO revealed convergence of altered pathways, similar to those induced by TLE4 or IKZF2 KO, including the upregulation of BCAT1, EGR1, and IL17RB (FIGS. 5A and 5B) and the acquisition of memory or effector over naïve T cell signatures (FIGS. 5C and 5D). However, targeting TMEM184B or EIF5A did not enrich for cytokine secretion and response pathways in CAR T cells (FIGS. 5E and 5F), which were found in TLE4-KO or IKZF2-KO CAR T cells. As these cytokines (CCL3 and CCL4) maintain T cell function during chronic viral infection and in the tumor microenvironment (57,58), our results indicate that TMEM184B-KO and EIF5A-KO CAR T cells might be prone to terminal effector differentiation and subsequent exhaustion, thereby compromising their overall functional capability despite their potent in vitro cytotoxicity. Overall, knockout of these genes in CAR T cells also maintained transcriptional profiles of T cell activation, which are associated with effector potency.

### Example 3: Targeting TLE4 and IKZF2 Modify CAR T Subsets Associated with Effector Potency

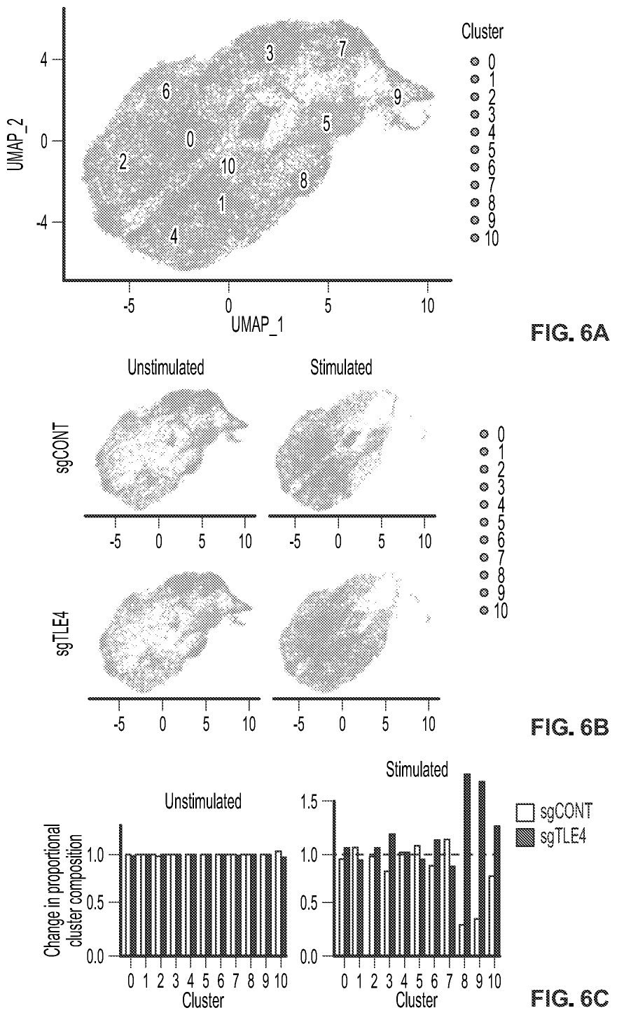

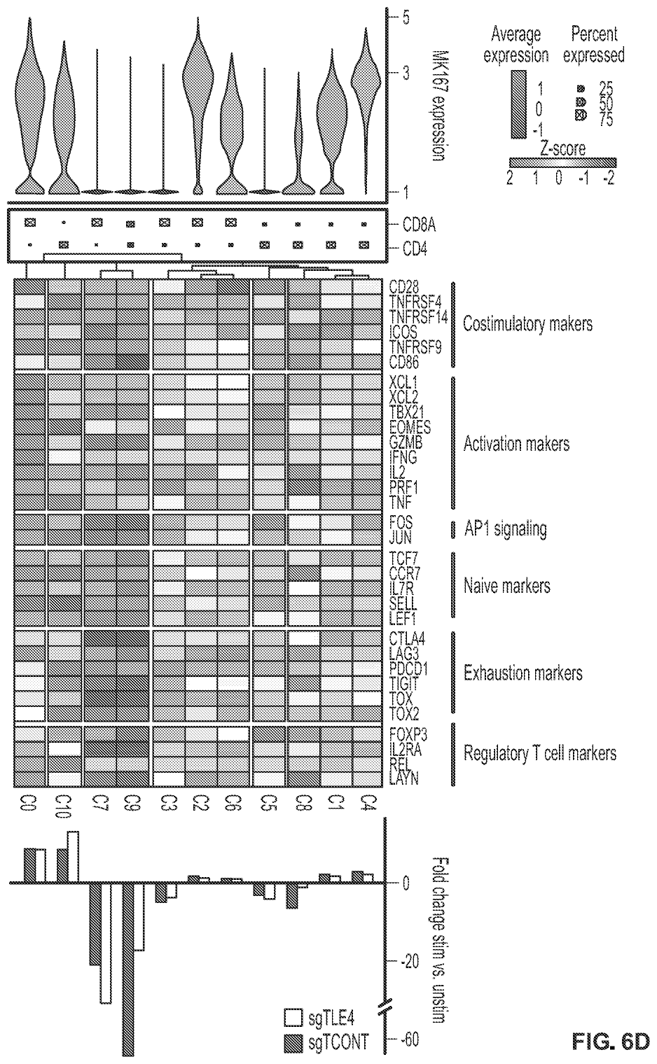

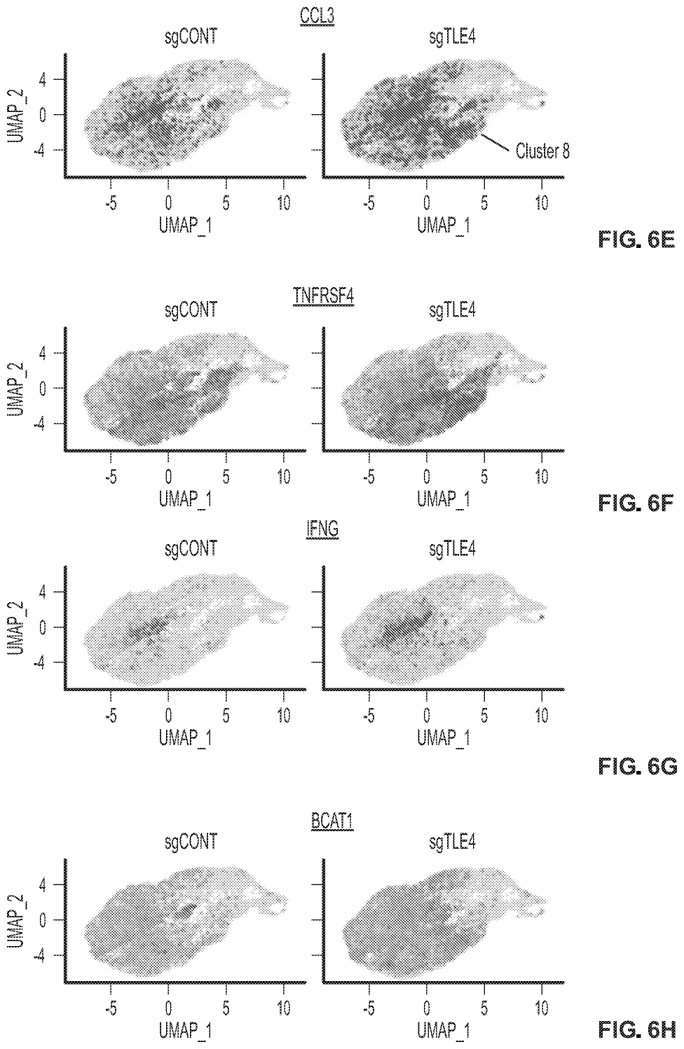

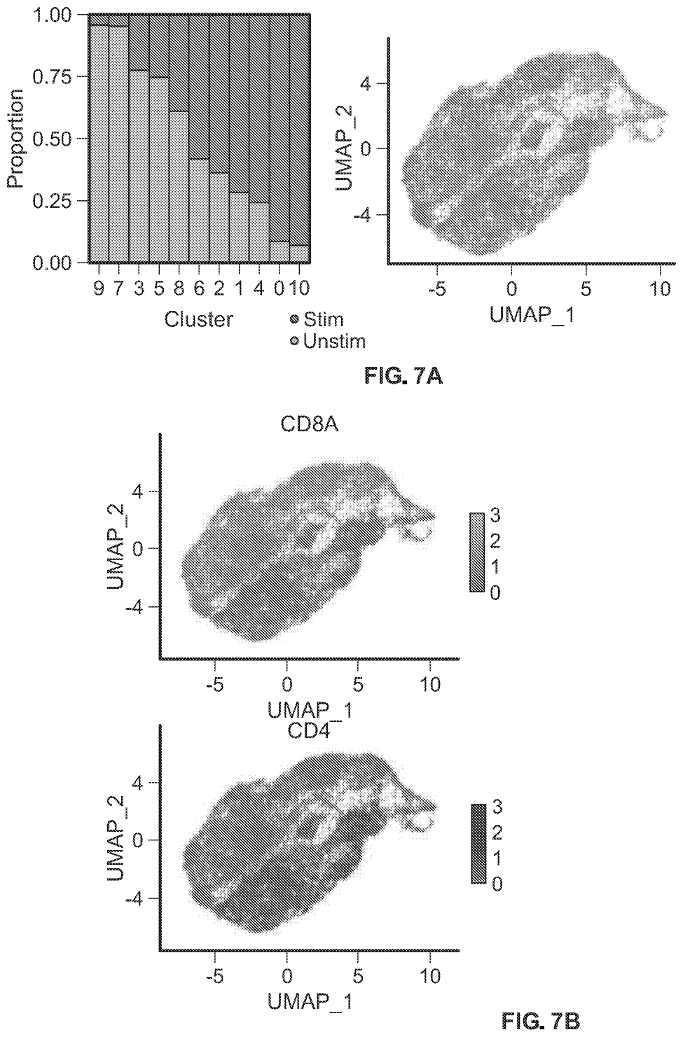

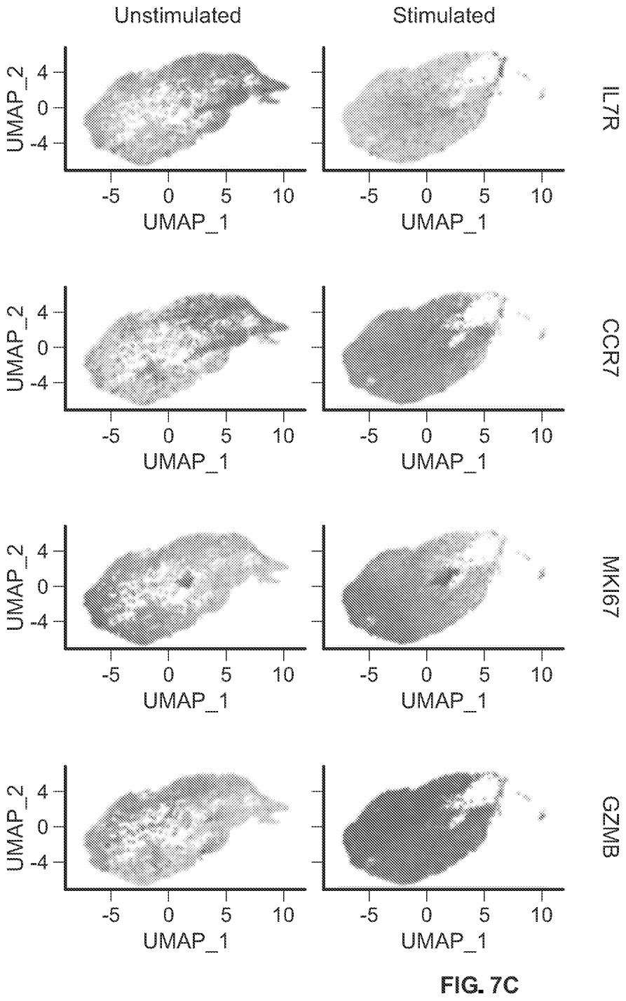

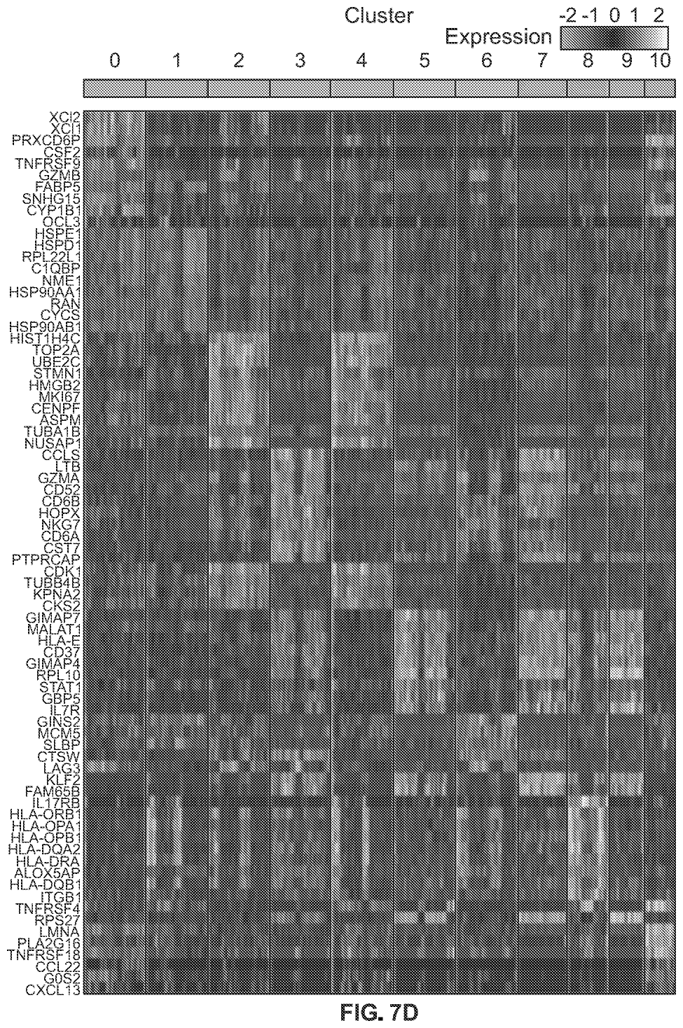

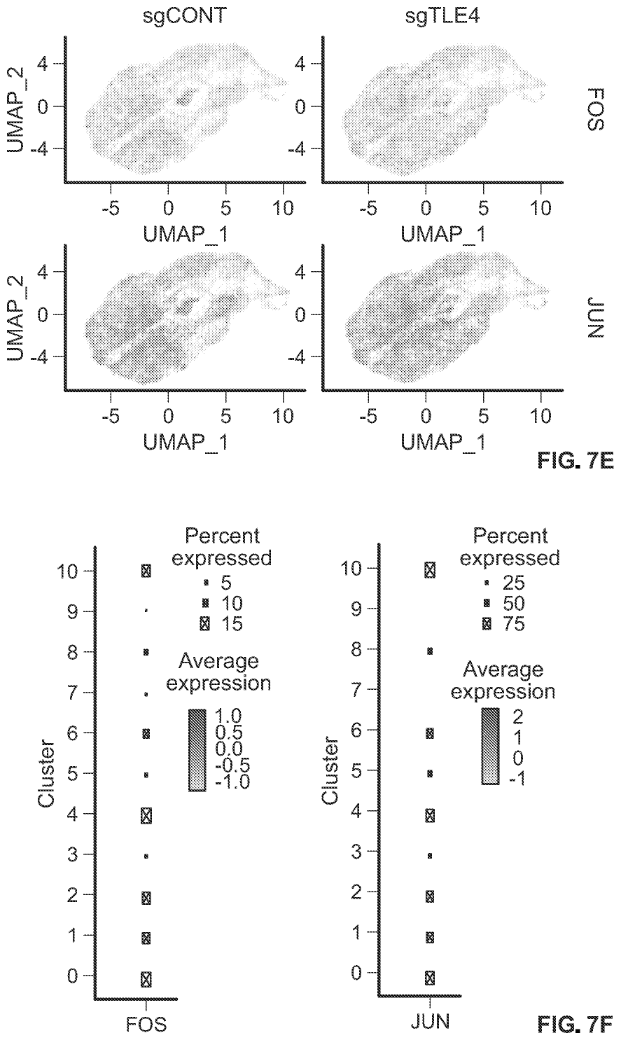

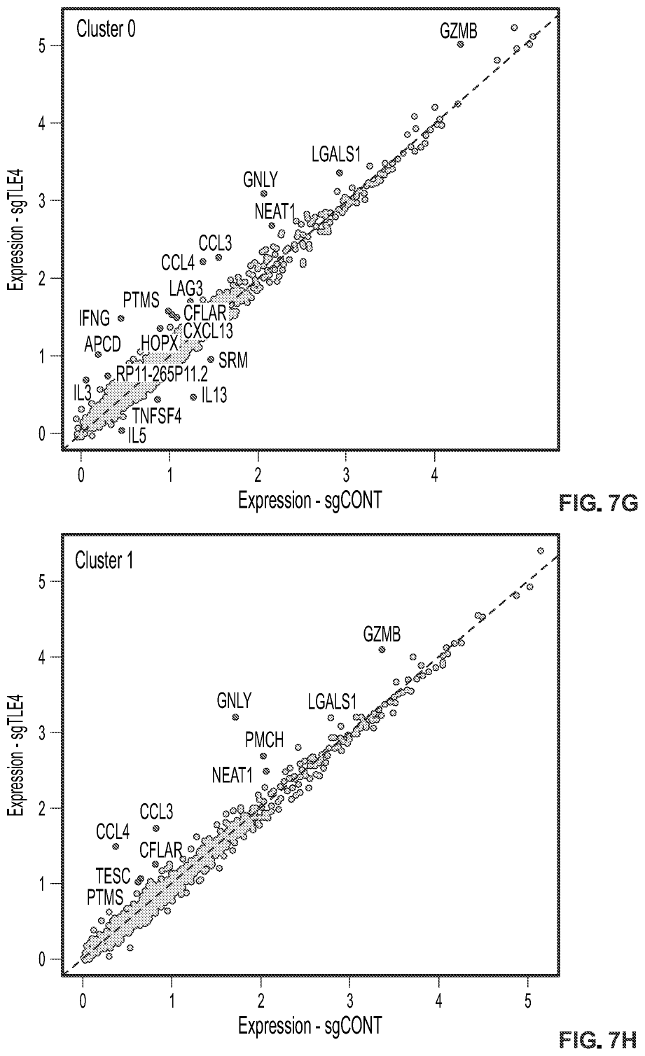

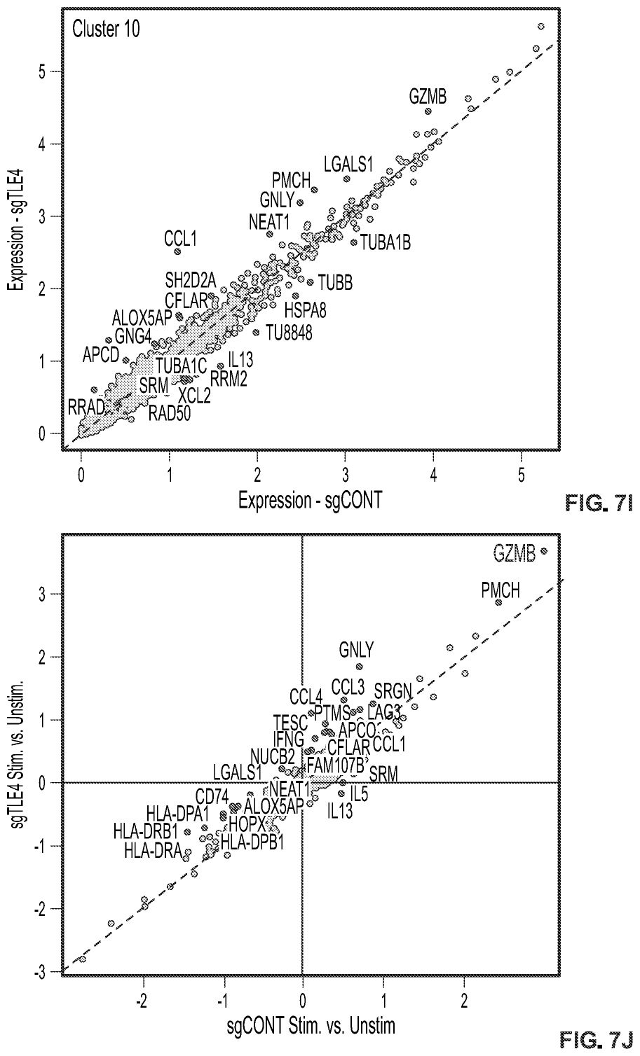

To determine the impact of TLE4 or IKZF2 KO on specific subpopulations of CAR T cells, we performed comparative single-cell RNA-sequencing (scRNAseq) on KO and control CAR T cells with or without stimulation by tumor cells. Comparing TLE4-KO cells with control CAR T cells by unbiased clustering of pooled data identified 10 different clusters, the distribution of which was greatly influenced by stimulation (FIG. 6A-C; FIG. 7A). CD4+ and CD8+ CAR T cells were well delineated (FIG. 7B). Stimulated cells downregulated naïve/memory-related markers (e.g. IL7R and CCR7) and upregulated activation-related markers (e.g. MKI67 and GZMB) (FIG. 7C). Stimulation enriched clusters 0, 1, 4, and 10 (showing high expression of activation or exhaustion markers) and depleted clusters 3, 5, 7, and 9 (expressing naïve/memory markers) (FIG. 6D). TLE4 KO minimally impacted the overall distribution of unstimulated CAR T cells; however, cluster 8 was depleted after stimulation only in control, but not in TLE4-KO cells (FIGS. 6C and 6D). This cluster represented a subset of CD4+ T cells expressing multiple costimulatory molecules, including CD28, ICOS, CD86, and TNFRSF4 (OX40), as well as the cytokine IL-2 (FIG. 6D; FIG. 7D). Although no proliferative activity was detected in this cluster (indicated by low Ki67), preservation of this cluster in TLE4-KO cells was maintained post-stimulation (FIG. 6D). In TLE4-KO cells, cluster 8 also showed expression of the immune stimulatory cytokine CCL3 (FIG. 6E), costimulatory molecule TNFRSF4 (FIG. 6F), and AP-1 transcription factors FOS and JUN (FIGS. 7E and 7F), which were minimally expressed in control cells. Cluster 10 was an activated CD4+ subset expressing multiple cytokines, including IL-2 and TNF, and this cluster displayed greater post-stimulation expansion in TLE4-KO cells (FIG. 6D). In the clusters with activation signatures (0, 1, and 10), TLE4 KO upregulated IFNG, BCAT, GZMB, CCL3, and CCL4 (FIGS. 6G and 6H; FIG. 7G-1). Combining the transcriptome readouts from all single cells revealed that tumor stimulation in TLE4-KO cells induced T cell stimulatory and cytotoxic factors (e.g. GZMB, CCL3, CCL4, and IFNG) to a greater degree than control CAR T cells (FIG. 7J). Taken together, the enhanced cytotoxicity of TLE-KO CAR T cells could result from the preservation of specific T cell subsets after tumor stimulation.

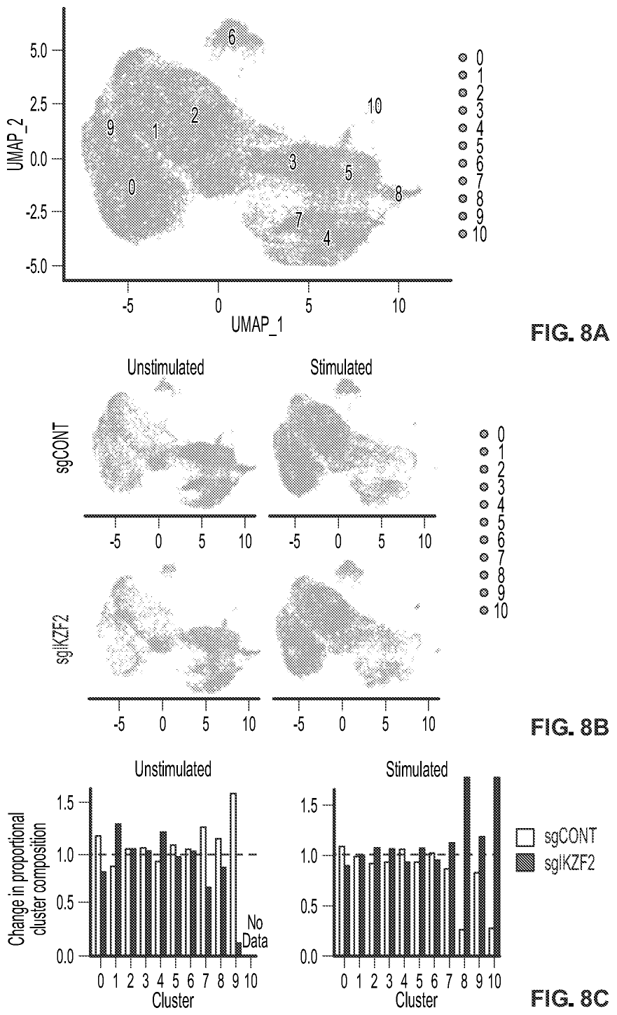

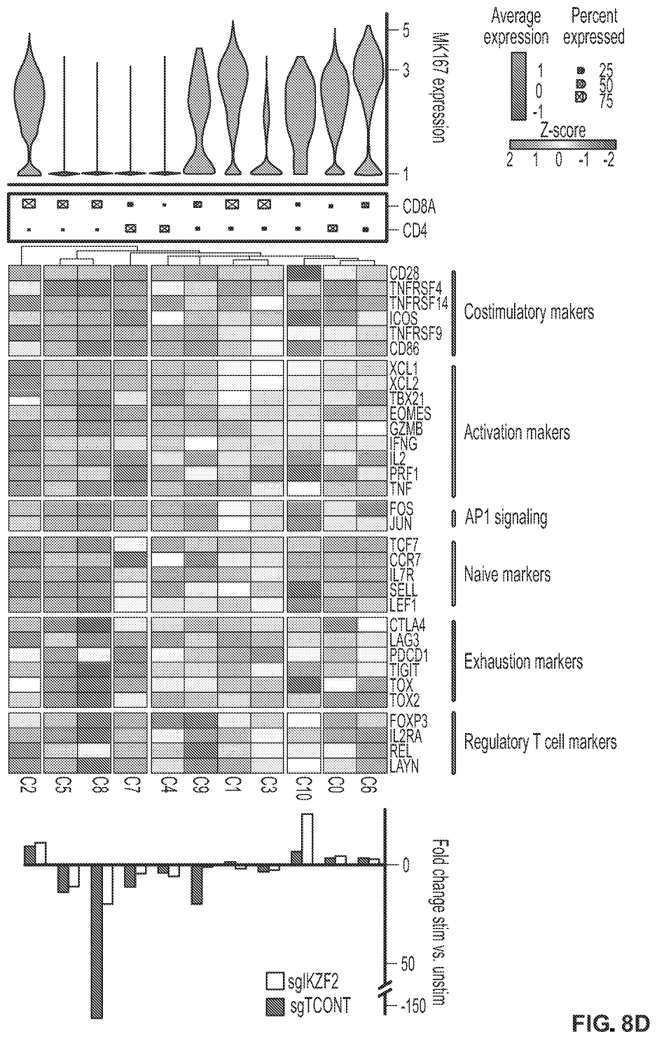

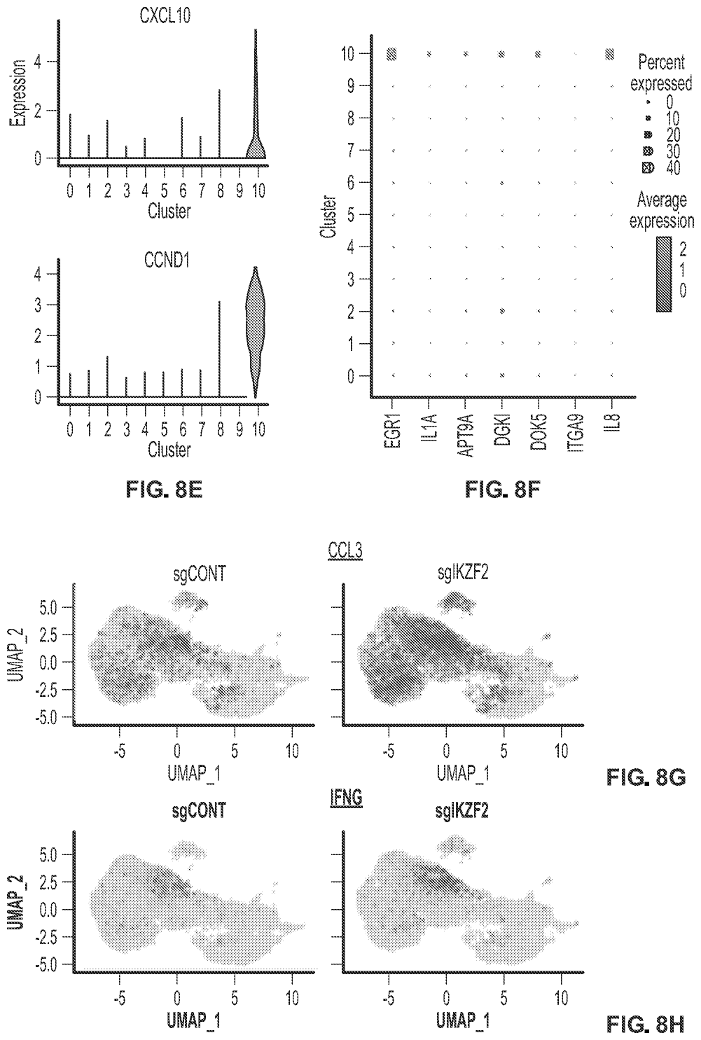

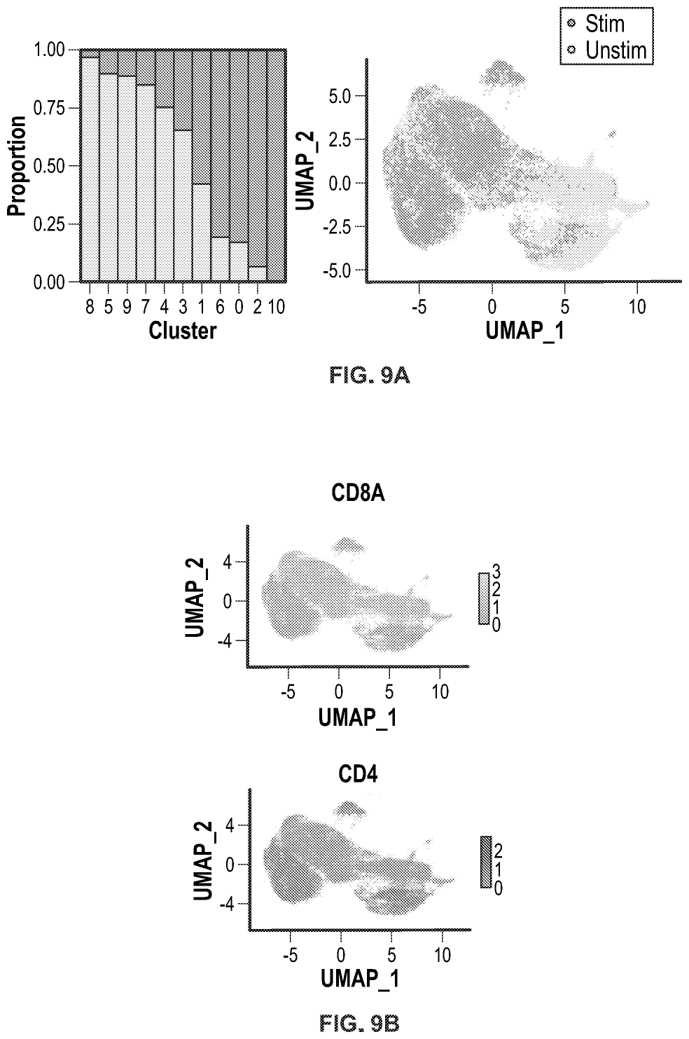

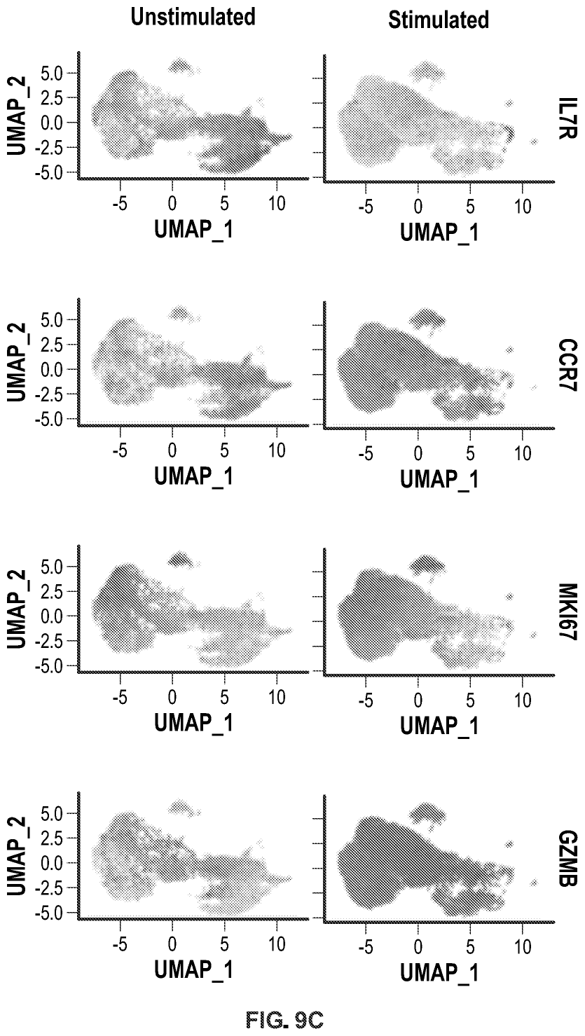

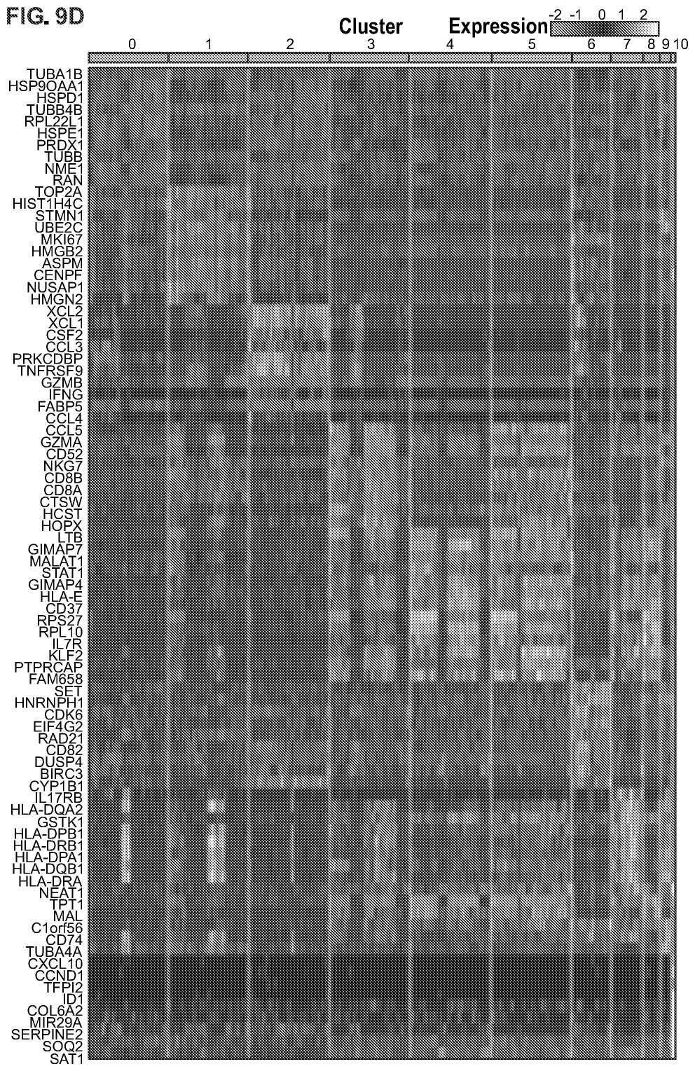

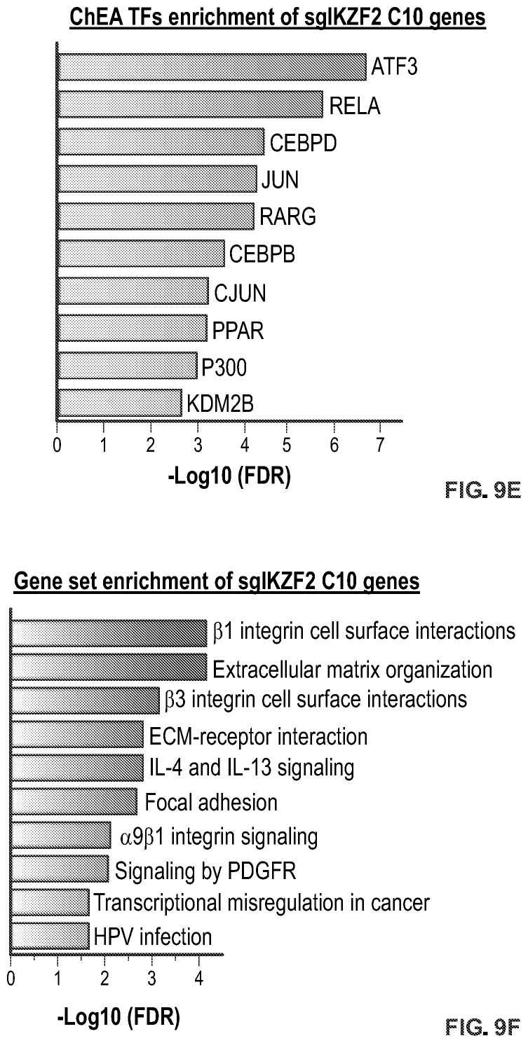

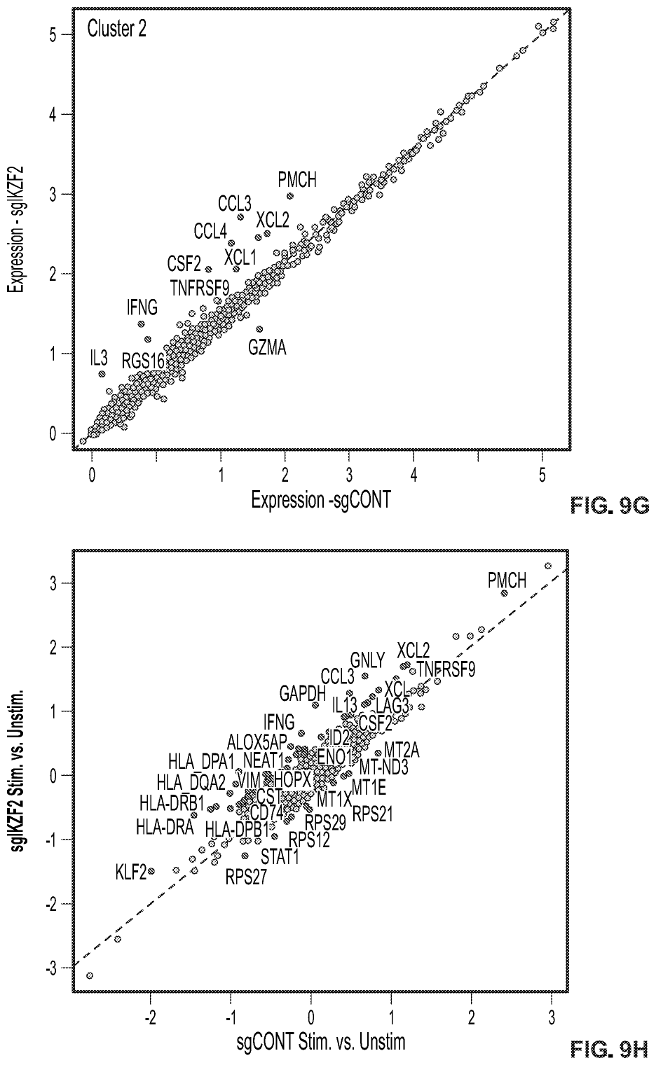

Comparison between IKZF2-KO cells and control CAR T cells identified 10 clusters using unbiased clustering of pooled data (FIG. 8A). In parallel with the comparisons between TLE4-KO vs. control cells, we observed a dramatic change in cluster distribution and gene expression after stimulation, with moderate changes from IKZF2 KO (FIGS. 8B and 8C; FIG. 9A-D). Given a role for IKZF2 in regulatory T cells (Treg) (59), cluster 0, characterized by Treg signatures (e.g. CLTA4, FOXP3 and IL2RA), was reduced in IKZF2-KO cells (FIG. 8B-D). Cluster 10 was induced after stimulation, enriched in IKZF2-KO cells, and expressed elevated levels of AP-1 signaling molecule FOS and JUN (FIG. 8B-D). These cells expressed a limited repertoire of cytokines beyond TNF, but had medium-to-high levels of Ki67, high expression of EGR1 and IL2, and exclusively expressed CXCL10 and CCND1 (FIG. 8D-F; FIG. 9D). Upregulated genes in cluster 10 were enriched for transcriptional regulation by ATF3 and JUN (FIG. 9E). This subset contained a very limited number of cells and was only present upon stimulation, potentially explaining the lack of differential expression of FOS and JUN in bulk RNA-seq analysis in IKZF2-KO cells. Cluster 2 was also expanded after stimulation in both IKZF2-KO and control cells (FIG. 8D; FIG. 9A). However, induction of activation-associated genes in this cluster, including IFNG, CCL3, and CCL4, was more robust in IKZF2-KO vs. control cells upon tumor stimulation (FIGS. 8G and 8H; FIG. 9G). In IKZF2-KO cells, CCL3 was expressed at higher levels in clusters 0, 1, and 9 (FIG. 8G). As a result, IKZF2-KO cells exhibited an augmented responsiveness to tumor stimulation, illustrated by the upregulation of activation-associated cytokines (FIG. 9H). Overall, scRNAseq analysis revealed that TLE4 or IKZF2 KO resulted in the preservation or expansion of certain CAR T cell subset after tumor stimulation. These cellular subsets displayed transcriptional signatures of T cell cytotoxicity and/or immune stimulation, providing some underlying mechanisms of their superior effector function against tumor cells.

### Example 4: Genome-Wide Screening of GSCs Identified Genes Mediating Resistance to CAR T Cells

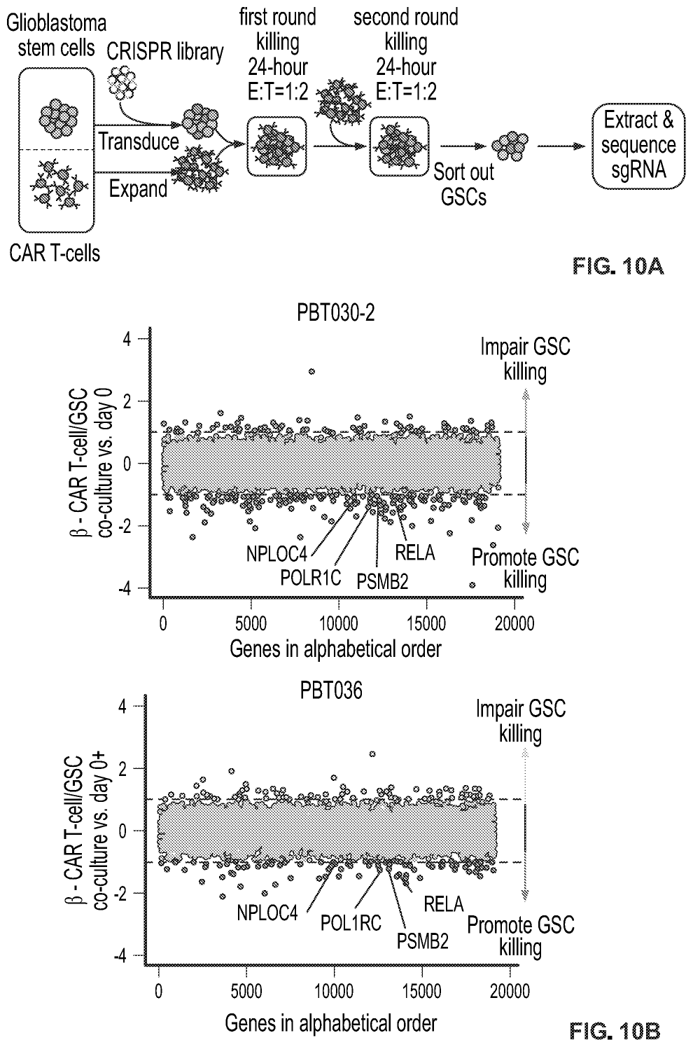

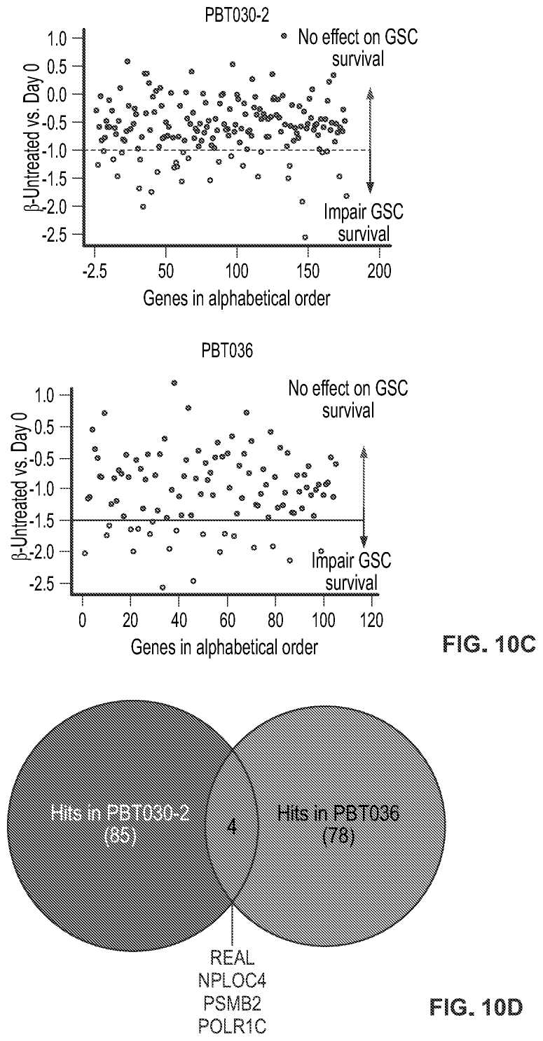

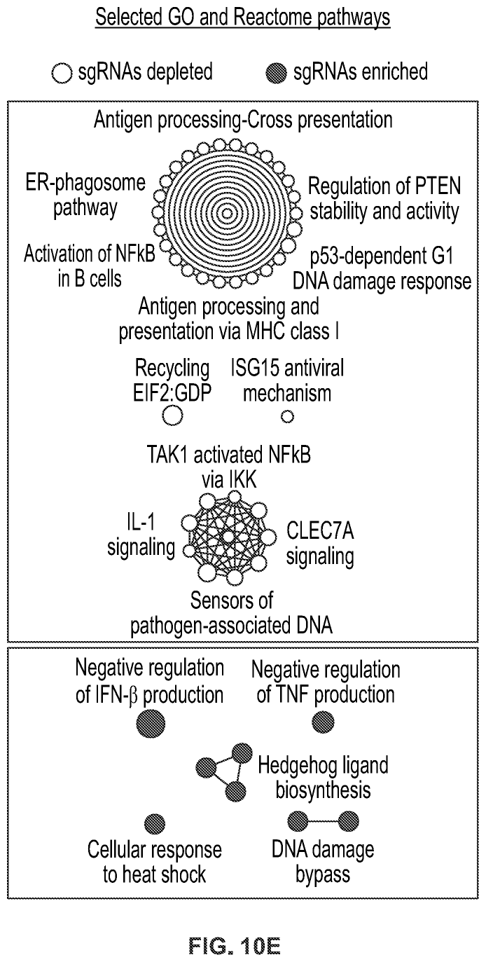

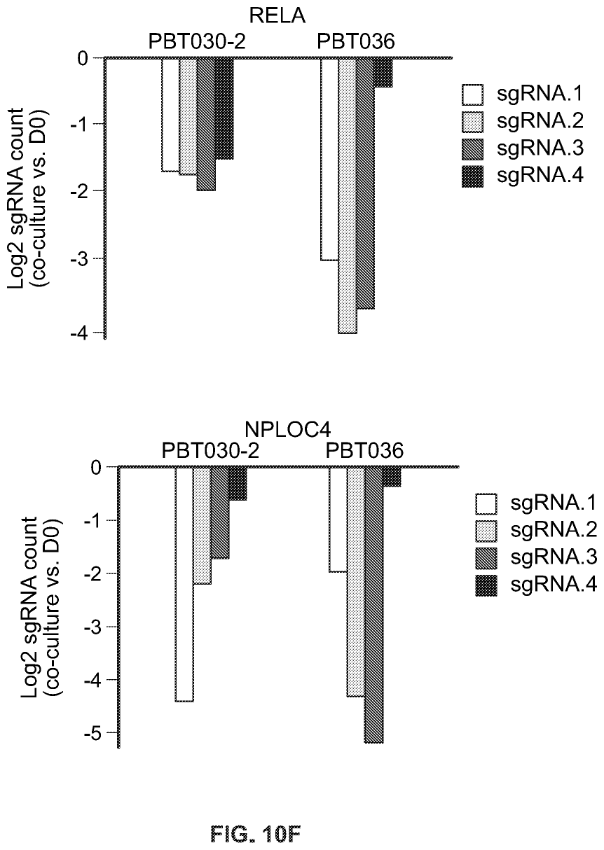

Augmenting efficacy of CAR T cells against GBM can be approached by studying T cells themselves, as above, which may inform targeted KOs in addition to CAR engineering for enhancing CAR activity. Reciprocal screening of GBM cells, especially GSCs, potentially informs interactions with CAR T cells to predict clinical responsiveness to CAR T cell therapy. To identify potential genes in GSCs that promote resistance to CAR-mediated cytotoxicity, we performed genome-wide CRISPR screens on two independent patient-derived GSC lines (PBT030-2 and PBT036), both derived from primary GBM tumors with high expression of IL13Ra2 (33). To identify tumor cell targets that rendered GBM cells more susceptible to T cell immunotherapy, we subjected GSCs to two rounds of co-culture with IL13Ra2-targeted CAR T cells (FIG. 5A). We identified sgRNAs that were enriched (β-value>1) or depleted (β-value<−1) in the surviving GSCs compared with GSCs in monoculture for the same amount of time (FIG. 5B). The genes with sgRNAs depleted in co-culture (β-value<−1) represented targets that promoted CAR killing upon knockout (FIG. 10C). To exclude sgRNAs that non-specifically targeted essential genes for GSC survival, we removed gene hits that were depleted in GSCs after 48-hour culture without CAR T cells (FIG. 10C). A total of 159 CAR-modulating genes were identified as hits in either GSC line, with only 4 overlapping targets common to both lines (FIG. 10D). Enriched pathways included tumor immune modulation, such as MHC I antigen presentation, IL-1 signaling and NF-κB activation (FIG. 10E), indicating that sgRNAs depleted in surviving GSCs targeted genes responsible for resistance to T cell killing.

### Example 5: Knockout of RELA or NPLOC4 Sensitizes GSCs to CAR-Mediated Antitumor Activity

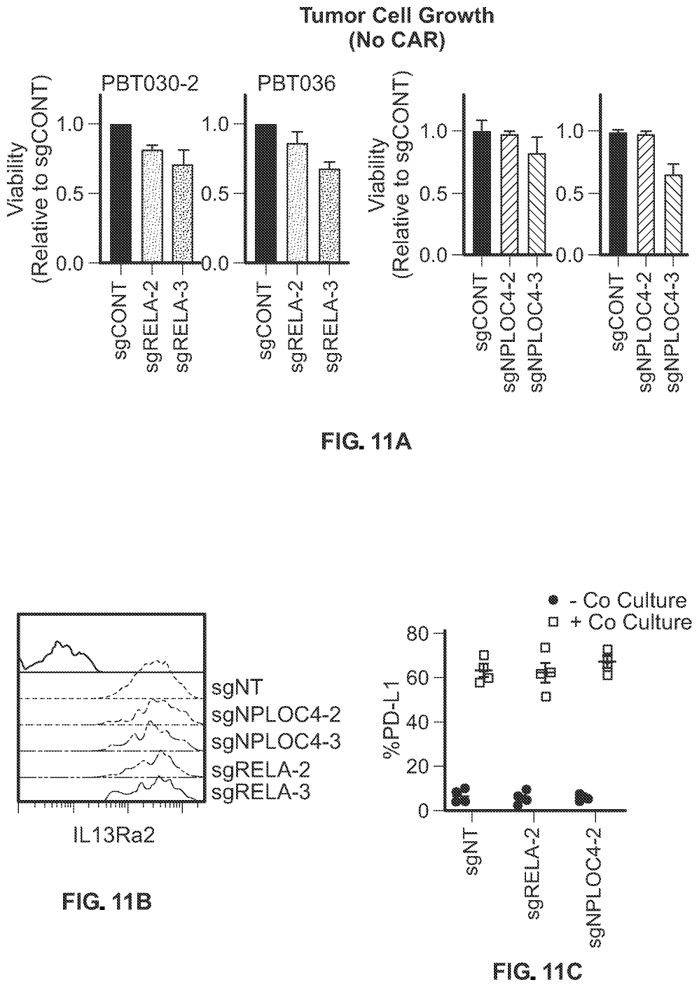

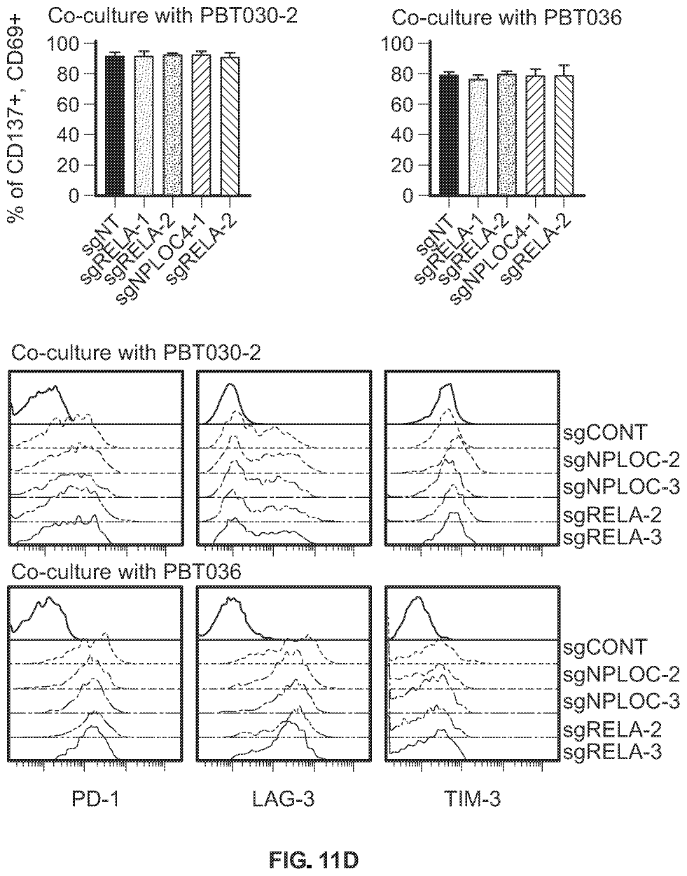

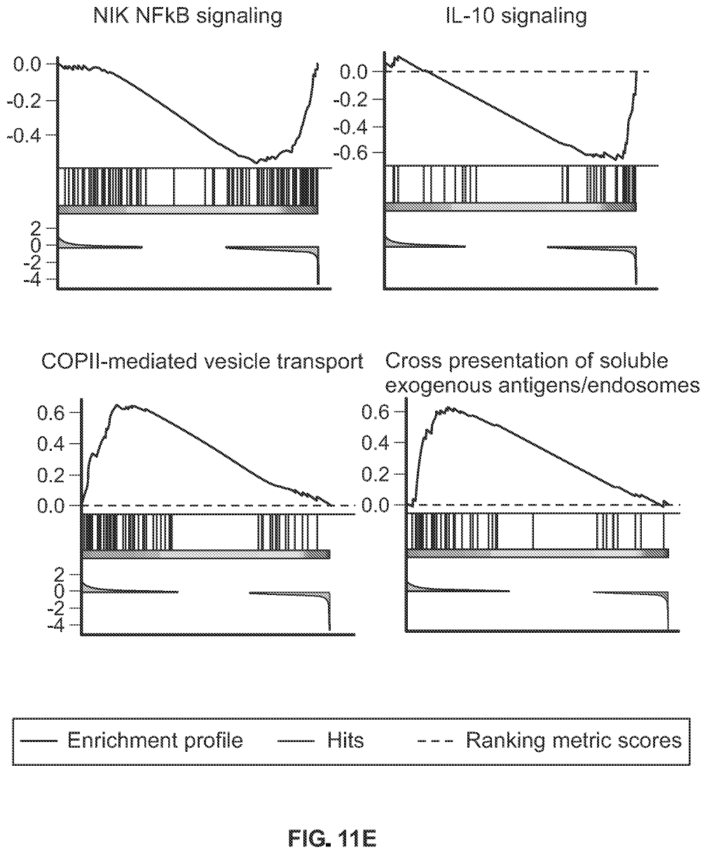

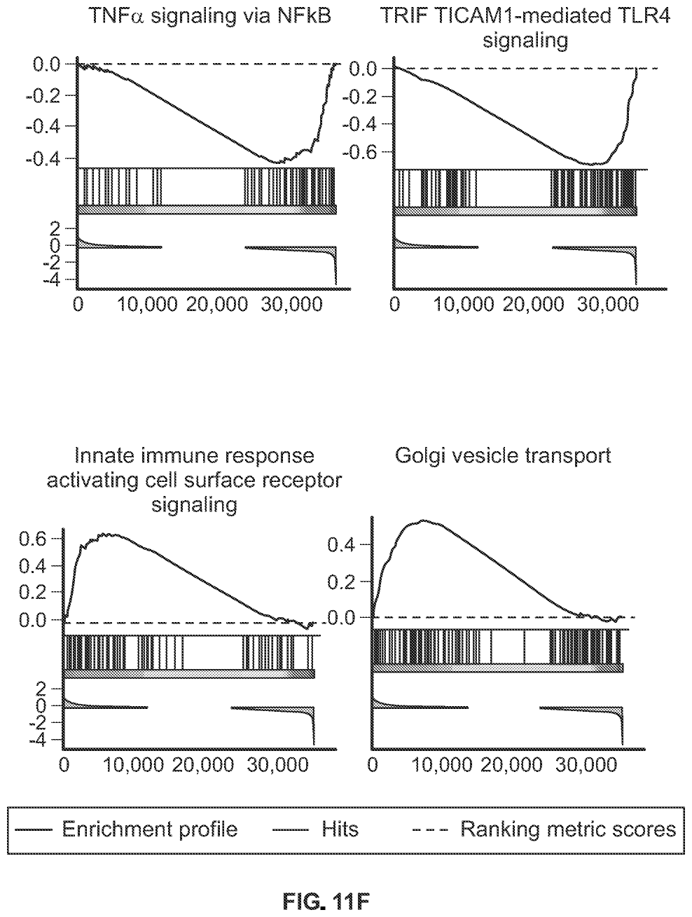

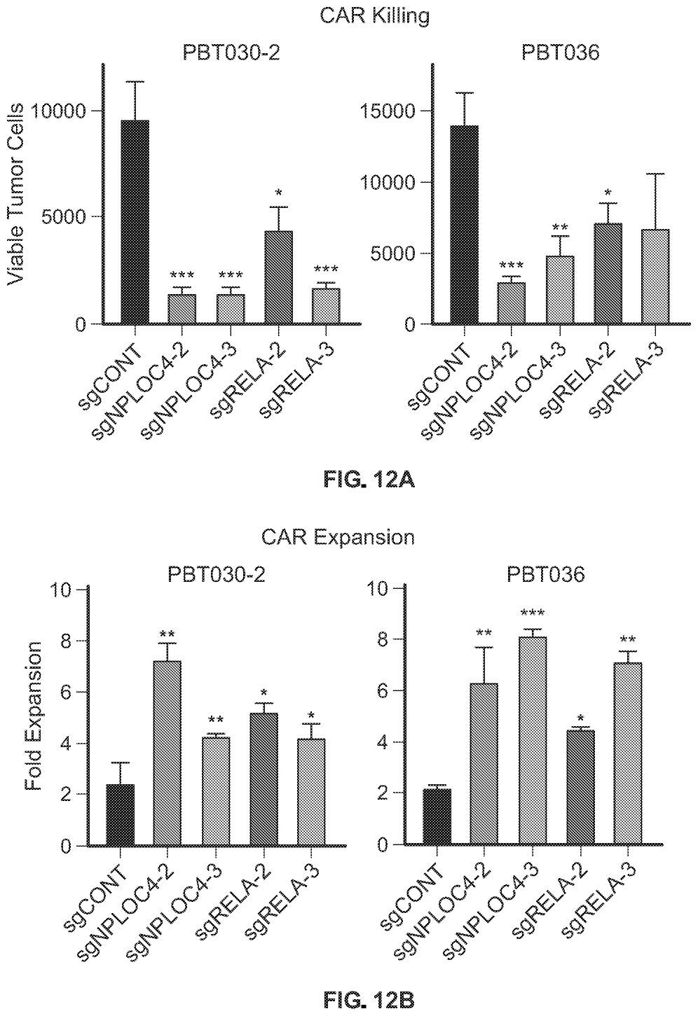

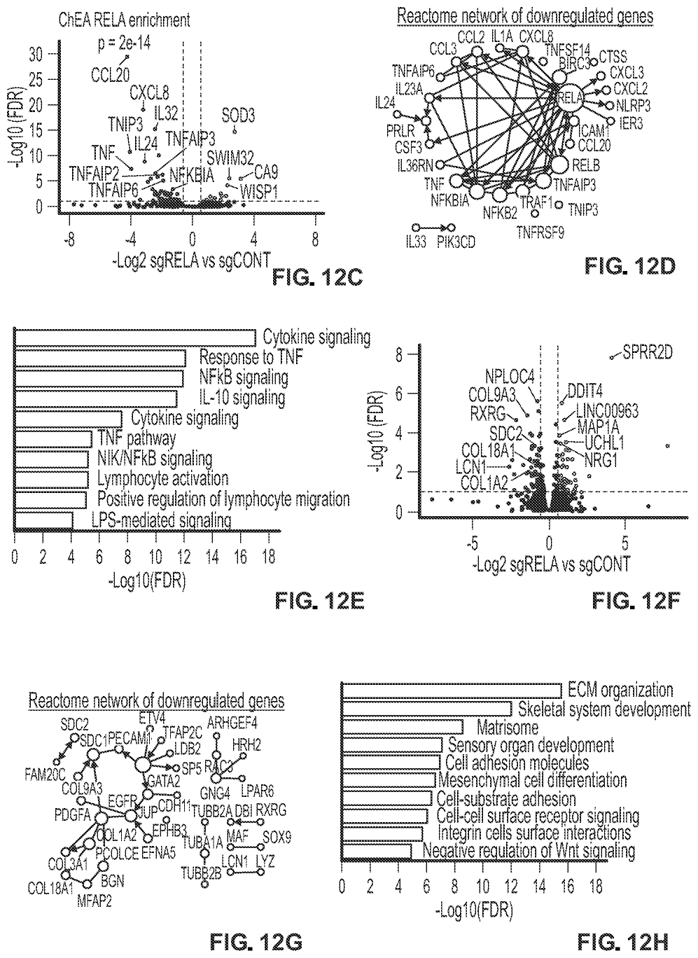

Next, we sought to confirm and further characterize the function of common top hits whose deletion promoted CAR killing (FIG. 10D). V-Rel Reticuloendotheliosis Viral Oncogene Homolog A (RELA) and Nuclear Protein Localization Protein 4 Homolog (NPLOC4) were selected for further validation as all sgRNAs targeting these two genes in the screen showed depletion in GSCs co-cultured with CAR (FIG. 5F). As expected from our selection process, CRISPR-mediated Knockout (KO) of either RELA or NPLOC4 caused limited reduction in the growth of GSCs in vitro compared with GSCs transduced with control non-targeted sgRNAs (sgCONT) (FIG. 11A). When co-cultured with CAR T cells in a challenging in vitro model at low T cell ratios (E:T=1:40; 48 hr), RELA or NPLOC4 KO in GSCs increased susceptibility to CAR T cell-mediated killing (FIG. 6A), which was also associated with increased expansion of CAR T cells (FIG. 12B). Thus, knockout of either RELA or NPLOC4 in GSCs enhanced the cytotoxic and proliferative potency of CAR T cells.

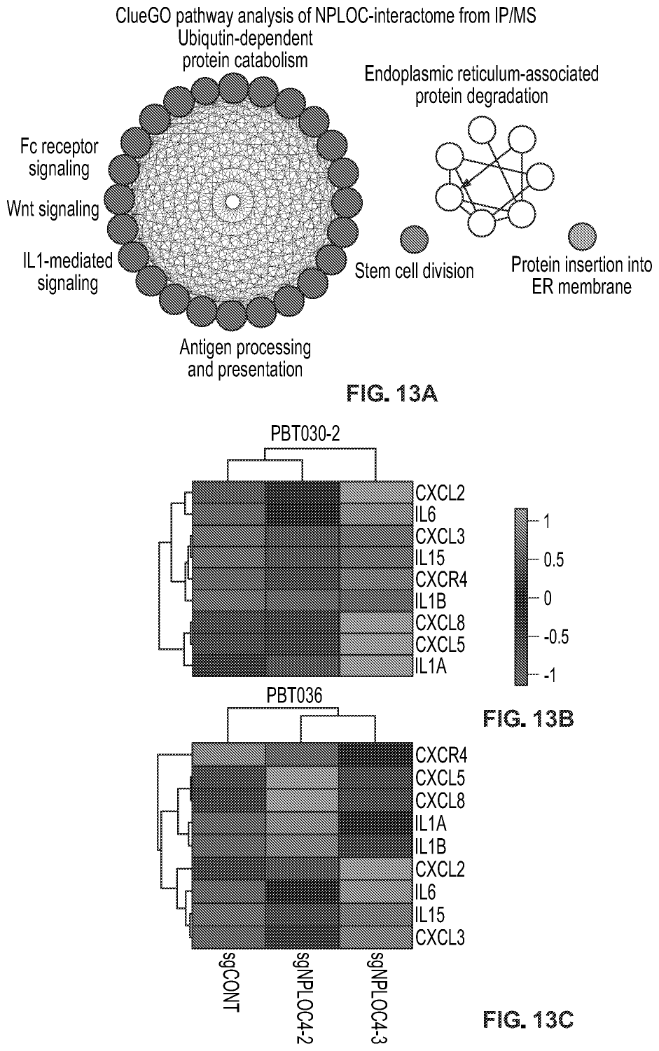

RELA (also known as p65) is an NF-κB subunit that regulates critical downstream effectors of immunosuppressive pathways in tumors (60,61). NPLOC4 mediates nuclear pore transport of proteins, but its role in cancer or immune modulation remains unclear. To elucidate the mechanism by which these genes mediate GSC sensitivity to CAR T cell killing, we performed in-depth characterization of GSCs harboring knockout of each gene. The increased sensitivity was not a result of alterations in target antigen expression on GSCs (FIG. 11B). CAR T cells induced PD-L1 in GSCs, which was not altered by depletion of either RELA or NPLOC4 (FIG. 11C). Likewise, CAR T cells co-cultured with GSCs transduced with sgCONT, sgRELA, or sgNPLOC4 did not show differences in activation after stimulation as indicated by markers CD69 and CD137, or exhaustion measured by levels of exhaustion markers, including PD-1, LAG-3, and TIM-3 (30) (FIG. 11D). Whole-transcriptome analysis of GSCs after RELA KO showed downregulation of immunosuppressive cytokines, including CXCL3, CCL20, and IL-32 (FIG. 12C), all of which suppress antitumor immune responses (62,63). Downregulated genes were highly enriched for known direct transcriptional targets of RELA, and RELA KO reduced NF-κB signaling, as well as the immunosuppressive effectors of TNF responsiveness and IL-10 signaling (FIGS. 12D and 12E; FIG. 11E). Targeting NPLOC4 in GSCs downregulated genes mediating rearrangement of extracellular matrix (ECM), including proteoglycans, integrins and collagens (FIG. 12F-H). Reactome analysis revealed the involvement of specific tumorigenic factors, such as EGFR and PDGFA (FIG. 12G). Pathways downregulated after NPLOC4 depletion were highly enriched for ECM remodeling and cell adhesion (FIG. 12H). Although tumor ECM remodeling has been reported to suppress antitumor immune responses by preventing T cell trafficking into the tumors, ECM-associated factors may directly repress T cell activity (64,65). To interrogate NPLOC4 interactions, we performed immune-precipitation followed by mass spectrometry (IP/MS), revealing that NPLOC4 bound multiple targets in immune-related pathways (IL-1, Fc receptor, antigen presentation), Wnt signaling, and protein synthesis/degradation pathways (FIG. 13A). These mechanisms may regulate the immune-related profiles of GSCs, where NPLOC4-KO led to the upregulation of immune stimulatory cytokines (FIGS. 13B and 13C). Together, we found that tumor-intrinsic regulators RELA and NPLOC4 mediate GBM resistance to CAR T cell cytotoxicity via mechanisms distinct from induction of CAR T cell exhaustion.

### Example 6: CRISPR Screening Identified Targets with Functional and Clinical Relevance in GSCs

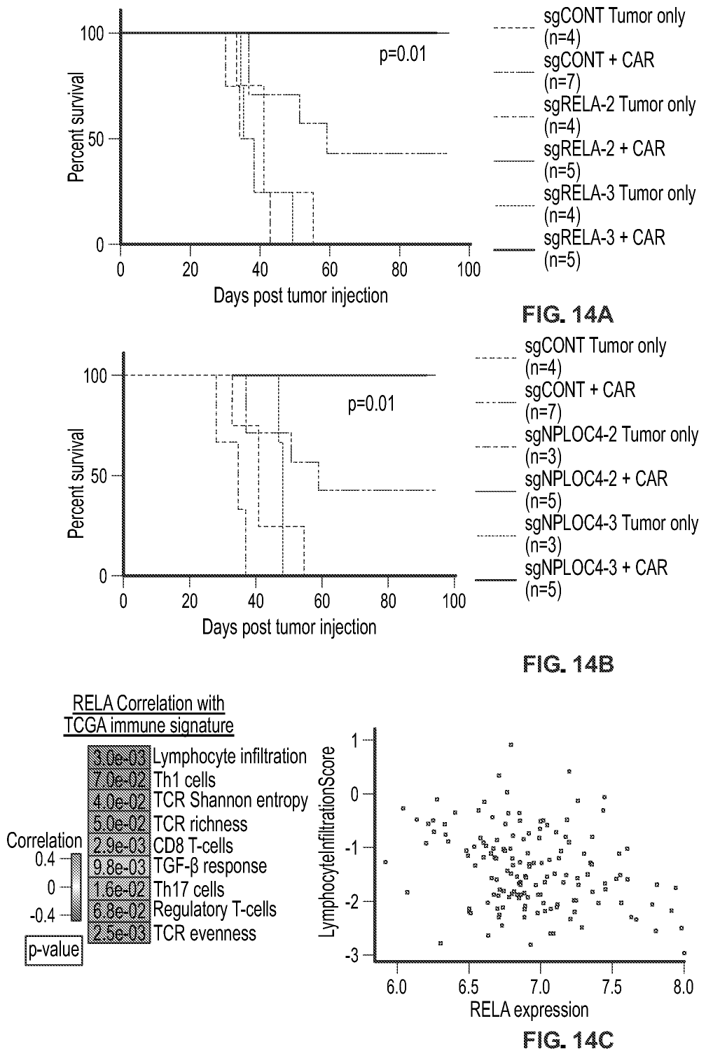

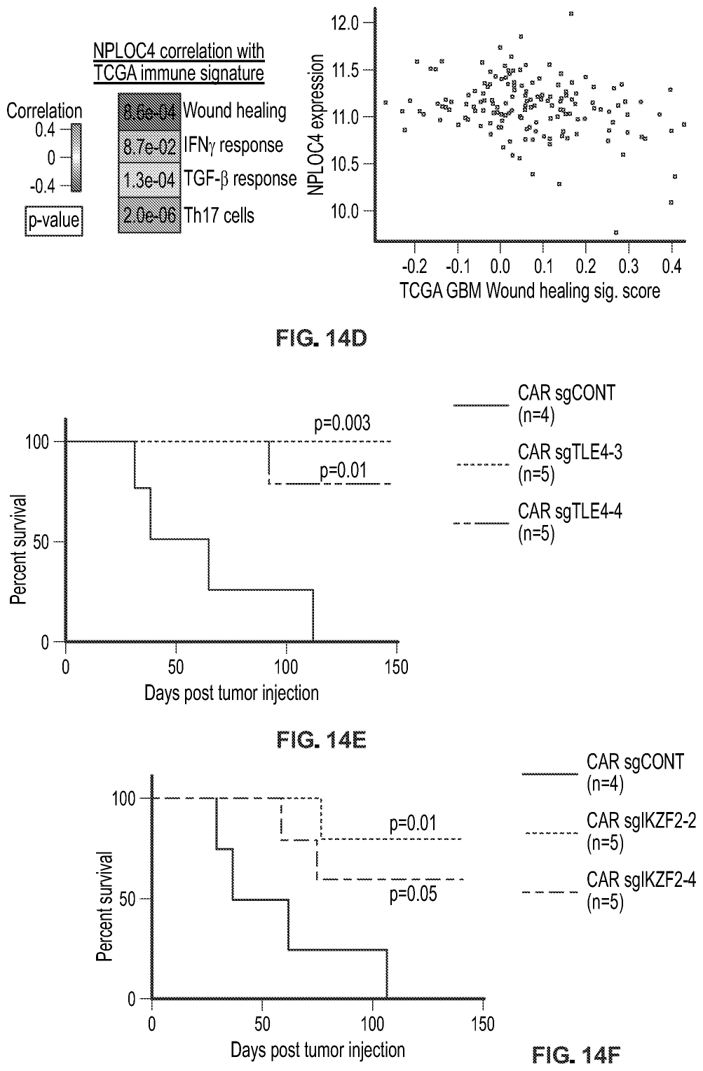

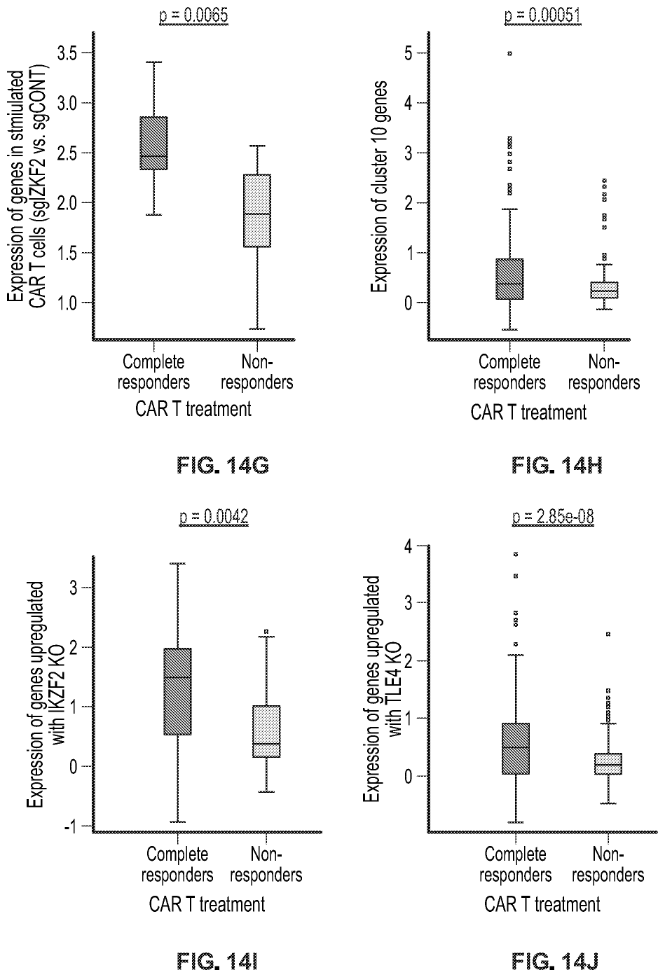

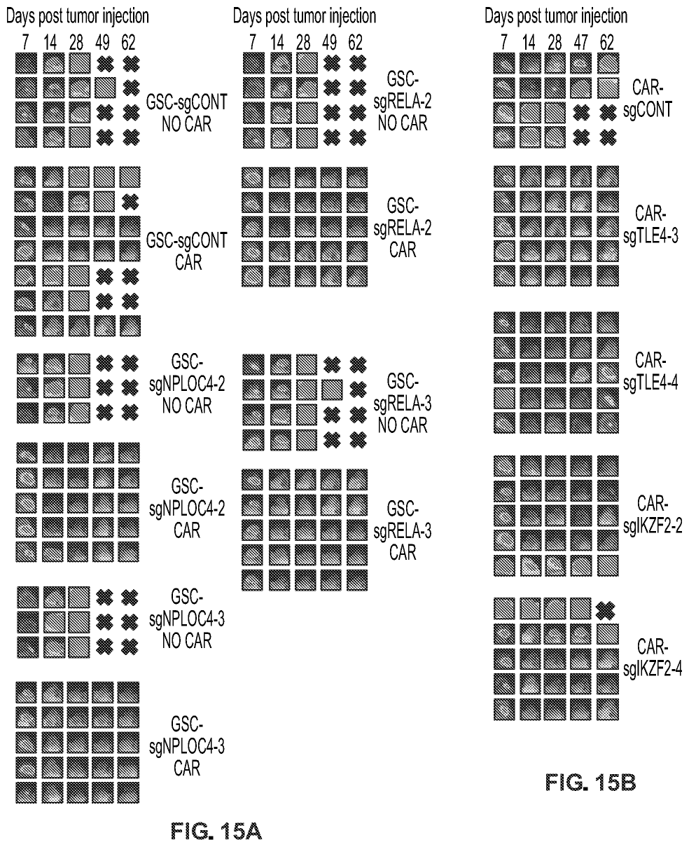

Next, we used an orthotopic intracranial patient-derived xenograft model to evaluate whether modulating the identified targets on GSCs enhanced the antitumor function of CAR T cells in a preclinical setting. Established GBM PDXs were treated with CAR T cells delivered intracranially into the tumors, mimicking our clinical trial design of CAR T cell administration to patients with GBMs (7,66). First, we used CAR T cells without CRISPR knockout to treat control, RELA-KO, or NPLOC4-KO tumors. A limited number of CAR T cells (50,000/mouse) completely eradicated xenografts derived from RELA-KO or NPLOC4-KO GSCs, whereas the same CAR T cells were only partially effective against tumors established with sgCONT-GSCs (FIGS. 14A and 14B; FIG. 15A). These results suggest that tumors with low expression of RELA and/or NPLOC4 are more sensitive to CAR T therapy.

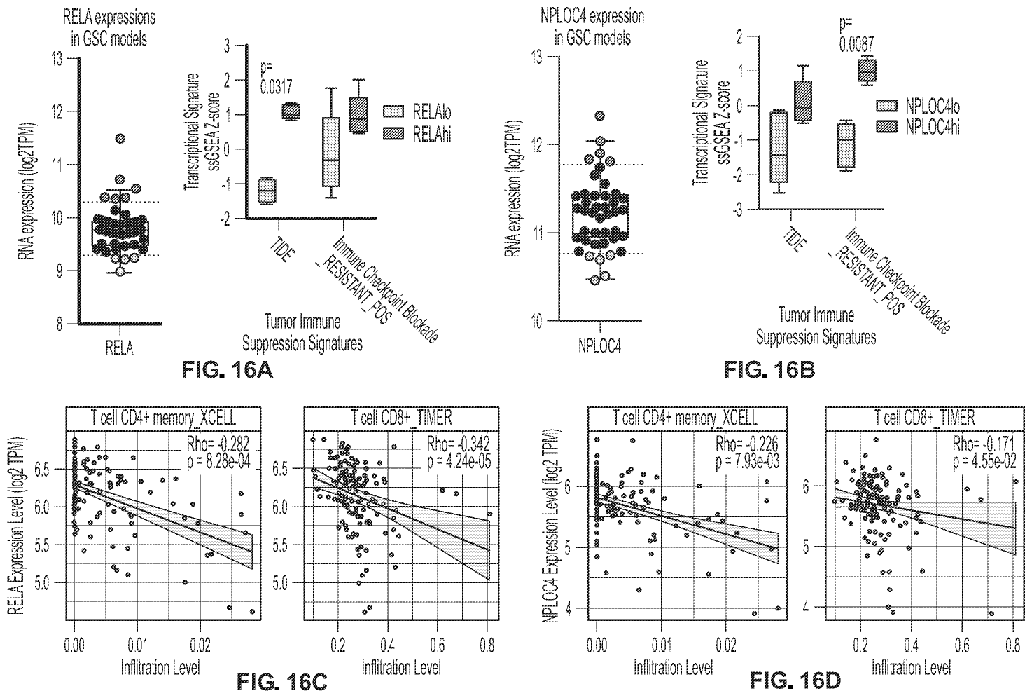

To further dissect the roles of RELA and NPLOC4 in immune modulation in GBM, we analyzed 41 GSC samples, and found that high RELA- or NPLOC4-expressing GSCs showed enrichment in immune-suppression signatures (FIG. 16A). Interrogating The Cancer Genome Atlas (TCGA) GBM dataset revealed that RELA and NPLOC4 both positively correlated with TGF-β signaling, a key pathway mediating immune suppression in GBMs and many other types of tumors (67). RELA was also positively correlated with immunosuppressive regulatory T cell signatures and negatively correlated with the signatures of antitumor T cell responses (lymphocyte infiltration, TCR richness, Th1 and CD8 T cells) (FIG. 14C). NPLOC4 was negatively correlated with the immune stimulatory IFNγ responses (FIG. 14D). The infiltration signature of CD4+ and CD8+ T cells in GBM inversely correlated with RELA or NPLOC4 expression (FIG. 16B). These results suggest that high expression of RELA and NPLOC4 in GBM are indicative of a more suppressive tumor immune microenvironment, and, repressed antitumor T cell responses.

### Example 7: CRISPR Screening Identified Targets with Functional and Clinical Relevance in CAR T Cells

We next evaluated the molecular targets identified in our CAR T cell screen in vivo, with the goal of establishing clinically translatable strategies to improve CAR T cell function. The antitumor function of different CAR T cells were tested against tumors without CRISPR knockouts, with a further limited CAR T cell dose (20,000/mouse) showing enhanced survival benefit as compared to the control CAR T cells failed to achieve long-term tumor eradication (FIGS. 14E and 14F). Consistent with improved maintenance of T cell effector activity and decreased exhaustion, targeting either TLE4 or IKZF2 augmented in vivo antitumor activity of CAR T cells against PDXs, as measured by extension of survival in tumor-bearing mice (FIGS. 14E and 14F; FIG. 15B). Depletion of TMEM184B or EIF5A in CAR T cells showed a trend towards improved efficacy in increasing the survival of tumor-bearing mice (FIGS. 17A and 17B). Therefore, these targets on GSCs and CAR T cells can be exploited to advance the efficacy of CAR therapy against established GBM tumors.

We then investigated whether the CAR T cell targets indicate the potency of clinical therapeutic products. We then mapped upregulated genes in IKZF2-KO CAR T cells compared to control CAR T cells after tumor stimulation, with the transcriptomes of CAR T cell products from patients with chronic lymphocytic leukemia (CLL) achieving complete responses (CR) or no responses (NR) (27). Supporting our results, these genes were induced to a greater degree after CAR stimulation in the products from patients achieving CR (FIG. 14G). Similarly, genes enriched in cluster 10, whose expansion was induced by tumor stimulation and further augmented with TLE4 KO, were also highly expressed in the products from patients with CR (FIG. 14H). Further, both TLE4- and IKZF2-KO led to gene upregulation similar to comparisons of products from patients with CR and NR (FIGS. 14I and 14J).

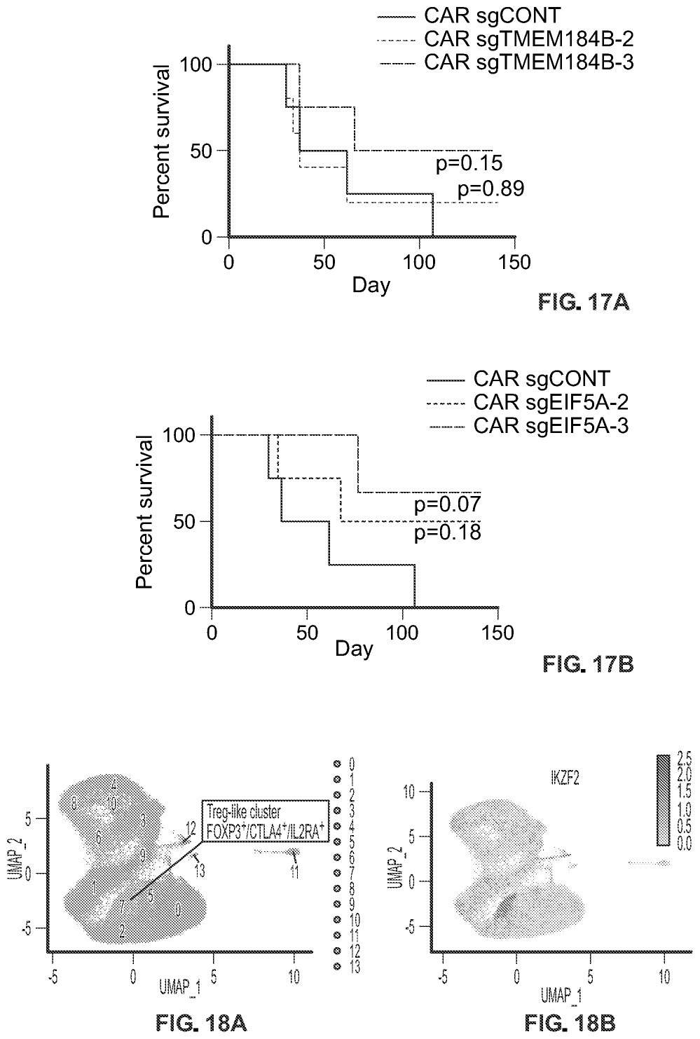

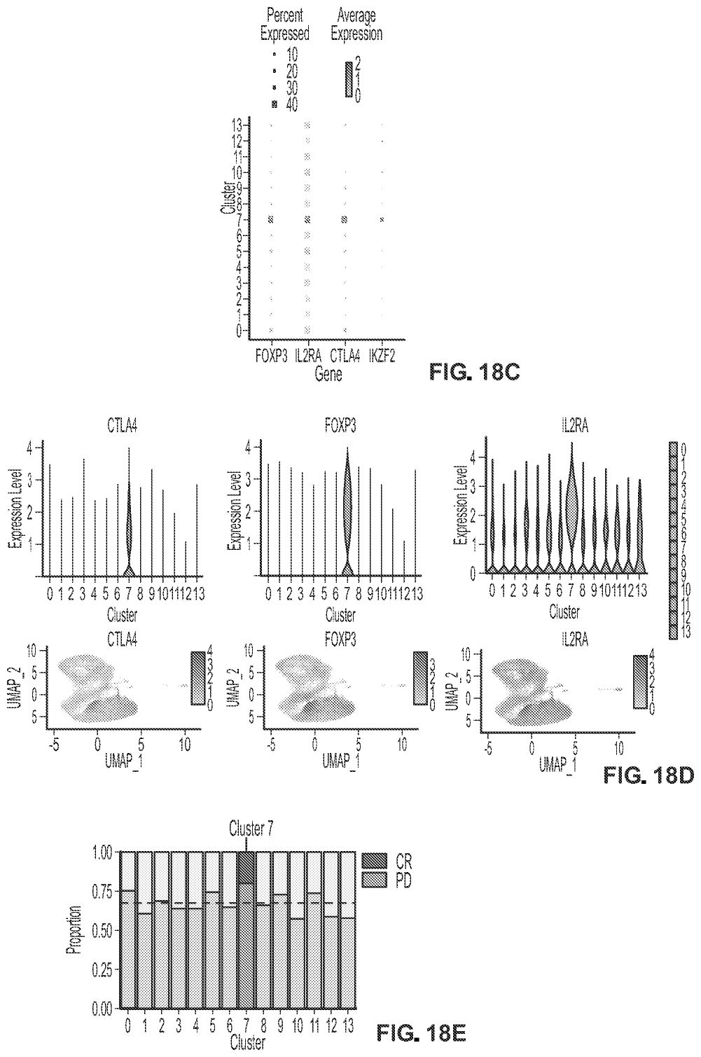

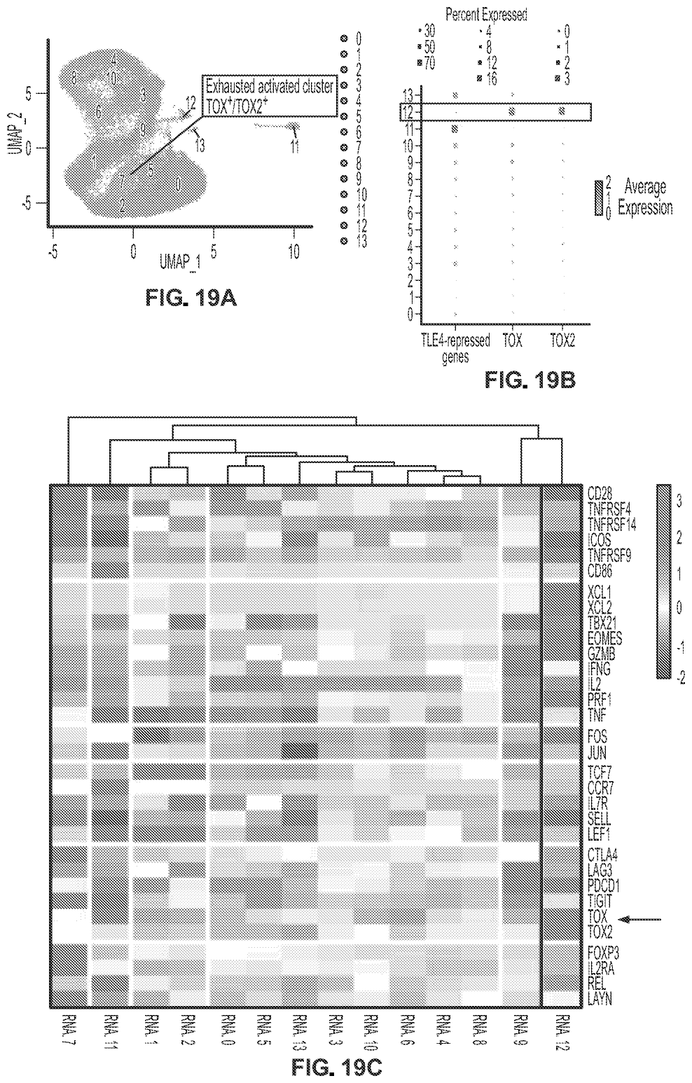

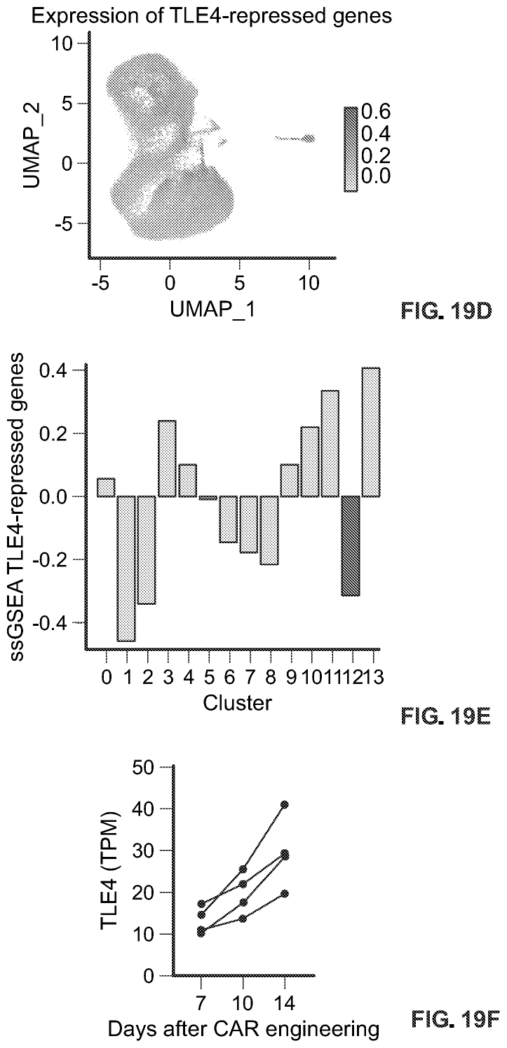

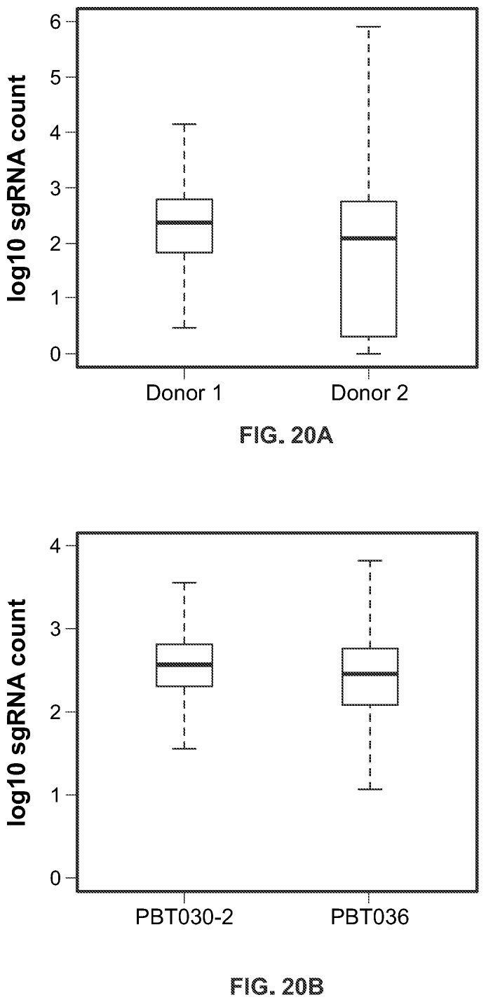

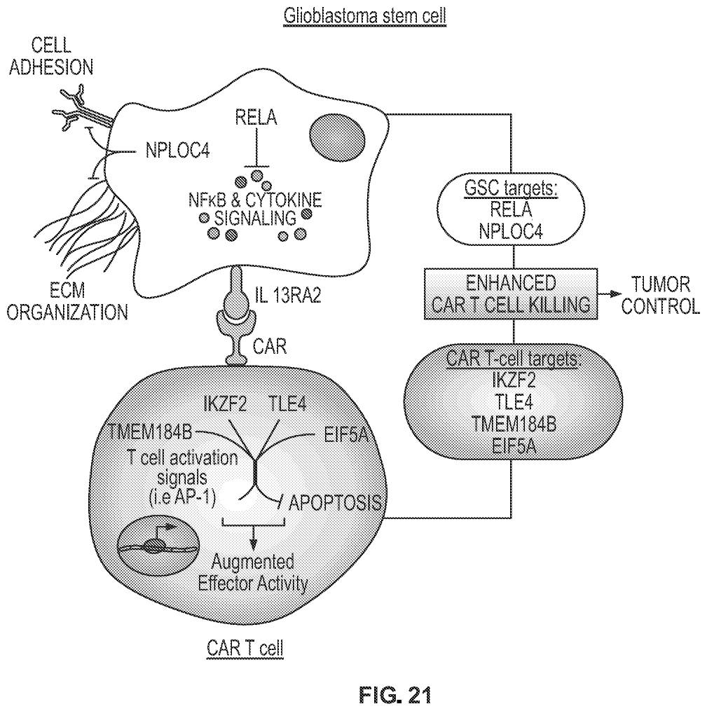

To further understand how TLE4 and IKZF2 contribute to the function of clinical CAR T cell products, we analyzed scRNAseq from 24 patient-derived CD19-CAR T cell products (68). An unbiased clustering of the scRNAseq data revealed that IKZF2 expression was highly enriched in cluster 7 (FIGS. 18A and 18B), overlapping with key markers of immune-suppressive Tregs (CTLA4, FOXP3, IL2RA; FIGS. 18C and 18D). Cluster 7 was more frequently detected in patients with progressive disease (PD) than those with complete responses (CR) (FIG. 18E). In these same cells, cluster 11 represented exhausted T cells, as indicated by the markers TOX and TOX2 (FIG. 19A-C) (37). This cluster showed low expression of TLE4-repressed genes, indicating high TLE4 activity (FIGS. 19D and 19E). Further, TLE4 was upregulated in CAR T cells undergoing extended ex vivo culture (FIG. 19F), a process associated with impaired effector function (69). Together, these observations establish that the targets identified from CRISPR screening have clinical implications for both tumor immunoreactivity and CAR T cell functional potency (FIG. 21).

### Example 8: Advantages and Additional Targets

T cell-based therapies may offer several advantages in GBM therapy. T cell-based therapies, especially when delivered into the cerebrospinal fluid (CSF), traffic to multifocal tumor populations within the central nervous system (CNS) (8,70-72), thus overcoming challenges associated with the blood-brain barrier that limits the CNS penetration of most pharmacologic agents. T cell therapies compensate for cellular plasticity within brain tumors more effectively than traditional pharmacologic agents. GBMs display striking intratumoral heterogeneity, and tumor cells readily compensate for targeted agents against specific molecular targets. With T cell therapy targeting different antigens, personalized treatments based on the antigen expression profile of individual tumors may be designed. T cell-based therapies induce secondary responses that augment endogenous anti-tumor responses. Adoptive cell transfer, especially CAR T therapies, have been investigated in clinical trials for GBM patients, but efficacy has been restricted to limited cases (11). Our focus on CAR T cells was prompted not only by the potential value for clinical translation, but also as our findings inform a broader understanding of T cell function in brain tumor biology.

Previous genetic screens used to identify interactions between immune cells and tumor cells have largely focused on the tumor cells (18,19,29), as these cells are easier to manipulate genetically. Screens on tumor-reactive mouse T cells have also been reported (20,73,74) given the establishment of Cas9-knockin mouse strain (75), as well as the convenience to acquire large numbers of these cells. Here, we interrogated both the human CAR T cell and tumor cell compartments. The screening strategy on CAR T cells was greatly facilitated by the development of the non-viral Cas9 expression system in primary human T cells (21). Here, the screening on tumor cells was performed on two independent GSCs, displaying a relatively narrow range of shared molecular targets involved in mediating responses to CAR T cells in our studies, which might be a consequence of subtype difference between these GSC lines (33). The screening identified both rational targets (RELA/p65) and novel targets (NPLOC4) in immune regulation, which were not restricted to a specific GBM molecular subclass. NPLOC4 displayed unexpected associations with GBM-targeting immune cell activity, as NPLOC4-KO in GSCs led to enhanced potency of CAR T cells and increased cytokine production in GSCs, although the detailed mechanism awaits further investigation. In the analyses of GSC models and TCGA database, high RELA and NPLOC4 expression was associated with immunosuppressive signatures. More specifically, higher expression of RELA and NPLOC4 in GBMs correlated with low infiltration of both CD4+ and CD8+ T cells, indicating that targeting these genes may confer immune modulatory effect and enhance antitumor T cell responses in GBMs.

The assay used for CRISPR screening in T cells is crucial for reliable readouts and is required for its sensitivity to differentiate effective versus non-effective therapies. Although the in vivo antitumor efficacy in mouse models has been the standard to evaluate the functional quality of T cells in adoptive transfer, the utilization of this system in screening has been controversial. Tumor-infiltrating T cells harvested after the injection of therapeutic cells display signatures of tumor reactivity (73) or, conversely, T cell exhaustion (40). The differential results appear model dependent, leading to mixed interpretation of the results. The co-culture assays that we used in this study identified key regulators by creating challenging screening environments. For the screening on GSCs, two rounds of short-term (24 h) killing with relatively large number of T cells (total E:T=1:1) was performed and GSCs were harvested immediately after the second round of killing, minimizing the effect of knocking out genes essential for the GSC growth. For the screening on CAR T cells, a repetitive challenge assay was used with excessive number of GSCs (total E:T=1:12), which we have shown to induce CAR T cell exhaustion (30). The screen was performed by comparing a less exhausted (PD1-negative) with a more exhausted (PD1-positive) subset, informing prioritization for maintenance of recursive killing function, while reducing the noise from tumor cell or T cell growth. The screening was performed with two independent CAR T cell donors, and the relatively small proportion of overlapping hits between the two donors was expected and consistent with previous studies (21,76), due to the variation in T cell populations between individuals. The target validation was done with different T cell donors and CAR platforms; therefore, the discovered immunotherapy targets may be generalizable to multiple CAR designs. While we validated 4 representative genes, the screening on CAR T cells resulted in over 200 potential targets involved in critical pathways of T cell biology and activation, offering additional targets for future investigation of CAR refinement. One limitation of our approach, however, is the exclusion of apoptosis pathways in tumor cells due to its critical role in tumor cell growth, which have been demonstrated as important regulators of CAR T cell-mediated tumor killing as well as tumor-induced CAR T cell exhaustion (29).

T cell exhaustion has been considered as one of the major hurdles for reducing CAR T cell potency (77-79). Blocking/knockout of inhibitory receptors is being rigorously investigated to augment CAR activity or other tumor targeting T cells (29,80,81). T cell exhaustion is a feedback mechanism after activation, occurring upon recursive exposure to antigens in the contexts of chronic infection or the tumor microenvironment (78,82) compromising their antitumor potency (79). Here, we observed that TLE4 or IKZF2 KO resulted in unstimulated CAR T cells to express transcriptional profiles of activation, while prohibiting exhaustion. AP-1 family transcription factors FOS and JUN, which were induced after both TLE4- and IKZF2-KO, provide a possible mechanism by which CAR T cell fitness was protected. The protein c-Jun forms homodimers or c-Fos/c-Jun heterodimers to initiate transcription of proinflammatory cytokines, and heterodimers with other co-factors (including BATF, IRF4, JUNB, and JUND) induce inhibitory receptors or suppress transcriptional activity of c-Jun (83-86). FOS was more upregulated than suppressive co-factors after TLE4-KO; therefore, driving T cell activation together with a protection from exhaustion, which was reminiscent of the effect after expressing c-Jun in CAR T cells with tonic signaling (55). In IKZF2-KO cells, however, the uncoupling of activation from exhaustion signatures was likely influenced by the upregulation of cytokines CCL3 and CCL4, which inversely correlated with PD-1 expression during T cell exhaustion (87). Both TLE4 or IKZF2 KO in CAR T cells upregulated essential regulators for Th1 cell differentiation (BCAT and EGR1, respectively), consistent with a previously identified role of this T cells population in mediating antitumor immunity (88,89). Consequently, targeted KOs in CAR T cells enhanced not only killing, but also expansion potential, which is correlated with clinical responses (90). Although it remains unresolved if these KOs potentiate CAR activity in immune-competent settings, our results have revealed the feasibility that CAR T cells can be modified for their activation/exhaustion signals to achieve functional improvement in clinically-relevant models. Consistent with these findings, we explored public databases of scRNAseq on patient-derived CAR T cell products and discovered that high IKZF2 expression and TLE4 activity were associated with other suppressive/exhaustion signatures of CAR T cells as well as poor clinical responses.

Single cell analyses reveal subset composition within a mixed cell sample, such as CAR T cells, in which minority populations serve critical roles. scRNAseq revealed that CAR activation, rather than genetic modification of CAR T cells (TLE4 or IKZF2 KO), resulted in a major cluster switch, which is consistent with the observation that TLE or IKZF2 KO in monoculture CAR T cells did not dramatically alter transcriptional profiles, as suggested by bulk RNA-seq. Following tumor challenge, knockout of targeted genes upregulated T cell activation markers and proinflammatory cytokines across different clusters, especially IFNG and CCL3, which showed similar induction by both TLE-KO and IKZF2-KO. Further, after CAR activation, TLE4 KO maintained a specific cluster, which existed pre-activation, and IKZF2 KO led to the emergence of a new cluster. The transcriptional signature of these clusters (expression of several costimulation molecules and cytokines) indicated their critical role in mediating effector function of CAR T cells. Therefore, the superior functions of TLE4-KO or IKZF2-KO CAR T cells were likely the result of a generally elevated activation state, as well as the stimulatory effect from critical subsets. Our scRNAseq results also suggested the existence of Treg-like populations, the expansion of which was seen after CAR activation and can be reduced by IKZF2-KO. The suppressive function of these cells still requires further investigation, but these results indicate the potential of enhancing CAR function through inhibiting differentiation towards Treg-like cells. Both TLE4-KO and IKZF2-KG CAR T cells appear to modify specific CD4+ T cell subsets, which supports our previous observation that CD4+ CAR T cells play a critical role in mediating potent effector function (30).

### Additional T Cell Gene Targets

Additional genes that can be knocked out in T cells harboring a CAR to improve CAR T cell function can include.

| Gene | Enrichment | Gene | Enrichment | Gene | Enrichment | Gene | Enrichment |
|---|---|---|---|---|---|---|---|
| SEL1L3 | 3.15565 | TIMM22 | 1.8449 | DNAH11 | 1.55245 | AADAT | 1.27385 |
| RXRG | 2.77905 | PIGR | 1.83365 | CARD16 | 1.54205 | DNAH5 | 1.2674 |
| EIF5A | 2.773 | ATP6V1E2 | 1.8199 | EGFL7 | 1.53965 | TSSK3 | 1.26545 |
| C14orf166 | 2.75935 | GNRHR | 1.81665 | ST7L | 1.5348 | SMR3B | 1.25785 |
| MLC1 | 2.71775 | GPR83 | 1.8162 | MLLT4 | 1.52635 | C12orf42 | 1.257 |
| PSORS1C2 | 2.65995 | MMACHC | 1.8149 | TMEM95 | 1.52295 | MCUR1 | 1.25645 |
| COG7 | 2.6155 | JADE2 | 1.81365 | CCL8 | 1.49605 | RGPD3 | 1.246 |
| ZBTB10 | 2.467 | GPR20 | 1.8119 | HMMR | 1.4945 | GPR39 | 1.2433 |
| ERCC6L | 2.46515 | MAGEB1 | 1.80755 | GMEB2 | 1.4789 | SGCD | 1.2405 |
| SYK | 2.46395 | TCEANC2 | 1.8067 | ABCA2 | 1.472 | ZNF592 | 1.2371 |
| YPEL3 | 2.45765 | PAQR9 | 1.8047 | C4B | 1.47 | DHRS9 | 1.23215 |
| HTR4 | 2.42875 | ADPRH | 1.7967 | ST3GAL4 | 1.46945 | HUNK | 1.23115 |
| MME | 2.41685 | RNF222 | 1.79155 | LRFN2 | 1.4687 | RALBP1 | 1.22875 |
| CDNF | 2.36145 | PHF6 | 1.7733 | ZNF354C | 1.4542 | ZBTB48 | 1.2277 |
| PLXNA4 | 2.34925 | NCDN | 1.77005 | PCDH11X | 1.4502 | ZNF70 | 1.22385 |
| CHML | 2.2995 | RNF138 | 1.76685 | GLIPR1L2 | 1.44445 | CXCR3 | 1.22145 |
| TAC4 | 2.29825 | NDEL1 | 1.7655 | CEP120 | 1.43855 | FAM57A | 1.2199 |
| TMEM39B | 2.28175 | CFHR5 | 1.76405 | CCDC77 | 1.43785 | FARP1 | 1.2197 |
| TBX10 | 2.27345 | ATP2A1 | 1.7624 | ACSL1 | 1.4311 | CLSTN3 | 1.2123 |
| WARS | 2.2684 | DNASE1L1 | 1.7612 | ANTXR1 | 1.4308 | DDB2 | 1.21165 |
| APOL4 | 2.23055 | C14orf132 | 1.75275 | ICOS | 1.42615 | IL1RN | 1.21105 |
| DHX16 | 2.2013 | SLC43A3 | 1.74675 | CTNNAL1 | 1.4236 | LRRC49 | 1.21055 |
| CADM3 | 2.1881 | FBXO27 | 1.7368 | ERLIN2 | 1.42305 | FZD9 | 1.2082 |
| TLE4 | 2.18255 | RAB23 | 1.71885 | ARSJ | 1.4221 | CSH2 | 1.208 |
| SLC18B1 | 2.1752 | XIRP2 | 1.7147 | SNRPD3 | 1.42095 | OR13F1 | 1.2039 |
| RNASEL | 2.16995 | NLRP1 | 1.70695 | NMNAT1 | 1.41815 | DPYSL3 | 1.18675 |
| TMEM80 | 2.10835 | POLR3G | 1.6797 | SPATA4 | 1.4178 | C20orf141 | 1.18245 |
| SV2B | 2.1055 | FAM219A | 1.67465 | C7orf60 | 1.4033 | ARPC3 | 1.1788 |
| KLHL33 | 2.09825 | SNX29 | 1.6654 | ELK3 | 1.40115 | PTPRG | 1.17805 |
| TMEM184B | 2.09715 | NTPCR | 1.65555 | CYB5A | 1.3928 | ZNF99 | 1.1734 |
| DOT1L | 2.05795 | RBBP8NL | 1.6539 | BIRC6 | 1.38515 | TGFBR3L | 1.1726 |
| SLC35D2 | 2.04645 | CAMTA1 | 1.6529 | PRKAA1 | 1.38025 | RANBP6 | 1.1702 |
| EID3 | 2.0363 | NR0B1 | 1.65125 | ZNF235 | 1.37775 | OXGR1 | 1.1598 |
| SPG7 | 2.0277 | ASAP3 | 1.64915 | FAM25C | 1.3498 | PRORY | 1.15255 |
| CBWD2 | 2.0193 | TT12 | 1.64665 | MRPL11 | 1.3403 | BPI | 1.147 |
| ANKUB1 | 2.01795 | ZBTB41 | 1.6302 | ZSWIM8 | 1.339 | C4orf48 | 1.14665 |
| SLC35D3 | 2.01715 | LRP1 | 1.61915 | SLC44A5 | 1.33545 | DSPP | 1.14605 |
| CENPBD1 | 1.9954 | PYY | 1.6188 | LOC730183 | 1.32535 | TBPL2 | 1.14425 |
| RAB3A | 1.9727 | ANKRD28 | 1.616 | EIF2S2 | 1.3214 | PADI6 | 1.1437 |
| EAF2 | 1.97225 | ADAMTSL1 | 1.59735 | ERCC4 | 1.3193 | AKAP1 | 1.13805 |
| IKZF2 | 1.9635 | ADAL | 1.5968 | NR2E1 | 1.31535 | DEFB126 | 1.13135 |
| LSM5 | 1.9485 | C9orf172 | 1.59035 | PSMA2 | 1.31475 | ZNF141 | 1.1284 |
| SORBS2 | 1.9438 | ZSCAN1 | 1.5901 | CAMTA2 | 1.3113 | NUBP2 | 1.12765 |
| SMAD2 | 1.93745 | MORN3 | 1.5892 | GTF2H3 | 1.30825 | AGAP3 | 1.1209 |
| VWA9 | 1.93245 | PCDHB2 | 1.58755 | RPH3A | 1.30715 | DOPEY2 | 1.11875 |
| ZRANB2 | 1.93115 | LTB | 1.58465 | ZNF766 | 1.2979 | OR10V1 | 1.11265 |
| MRPL42 | 1.93085 | TSPAN5 | 1.5797 | INTS12 | 1.29365 | RPL19 | 1.10805 |
| EPPK1 | 1.92875 | FAM199X | 1.5794 | KPNA2 | 1.2872 | MYO1F | 1.10185 |
| SMG1 | 1.91575 | NT5C1A | 1.5745 | AMY2A | 1.28325 | MYH14 | 1.0942 |
| CAPRIN1 | 1.91475 | DNAJC16 | 1.57085 | CPNE5 | 1.28315 | SPHKAP | 1.08525 |
| KRBA2 | 1.89985 | TCP11L1 | 1.5663 | MAFG | 1.2828 | TBCE | 1.0817 |
| TNFAIP8L1 | 1.88815 | CLEC6A | 1.56605 | FILIP1 | 1.27905 | OR10T2 | 1.0479 |
| S100A5 | 1.8843 | KIAA2026 | 1.5625 | ZNF878 | 1.27765 | REP15 | 1.04175 |
| OR14C36 | 1.86325 | GCG | 1.5595 | EPGN | 1.27595 | TEC | 1.02885 |
| TBC1D22B | 1.8585 | C14orf93 | 1.55485 | FGF13 | 1.27465 | ZIC2 | 1.0158 |

### REFERENCES

1. Lim M, Xia Y, Bettegowda C, Weller M. Current state of immunotherapy for glioblastoma. Nat Rev Clin Oncol 2018; 15(7):422-42 doi 10.1038/s41571-018-0003-5.
2. Maude S L, Laetsch T W, Buechner J, Rives S, Boyer M, Bittencourt H, et al. Tisagenlecleucel in Children and Young Adults with B-Cell Lymphoblastic Leukemia. N Engl J Med 2018; 378(5):439-48 doi 10.1056/NEJMoa1709866.
3. Neelapu S S, Locke F L, Bartlett N L, Lekakis L J, Miklos D B, Jacobson C A, et al. Axicabtagene Ciloleucel CAR T-Cell Therapy in Refractory Large B-Cell Lymphoma. N Engl J Med 2017; 377(26):2531-44 doi 10.1056/NEJMoa1707447.
4. Brown C E, Starr R, Aguilar B, Shami A F, Martinez C, D'Apuzzo M, et al. Stem-like tumor-initiating cells isolated from IL13Ralpha2 expressing gliomas are targeted and killed by IL13-zetakine-redirected T Cells. Clin Cancer Res 2012; 18(8):2199-209 doi 10.1158/1078-0432.CCR-11-1669.
5. Ahmed N, Salsman V S, Kew Y, Shaffer D, Powell S, Zhang Y J, et al. HER2-specific T cells target primary glioblastoma stem cells and induce regression of autologous experimental tumors. Clin Cancer Res 2010; 16(2):474-85 doi 10.1158/1078-0432.CCR-09-1322.
6. Morgan R A, Johnson L A, Davis J L, Zheng Z, Woolard K D, Reap E A, et al. Recognition of glioma stem cells by genetically modified T cells targeting EGFRvIII and development of adoptive cell therapy for glioma. Hum Gene Ther 2012; 23(10):1043-53 doi 10.1089/hum.2012.041.
7. Brown C E, Badie B, Barish M E, Weng L, Ostberg J R, Chang W C, et al. Bioactivity and Safety of IL13Ralpha2-Redirected Chimeric Antigen Receptor CD8+ T Cells in Patients with Recurrent Glioblastoma. Clin Cancer Res 2015; 21(18):4062-72 doi 10.1158/1078-0432.CCR-15-0428.
8. Brown C E, Alizadeh D, Starr R, Weng L, Wagner J R, Naranjo A, et al. Regression of Glioblastoma after Chimeric Antigen Receptor T-Cell Therapy. The New England journal of medicine 2016; 375(26):2561-9 doi 10.1056/NEJMoa1610497.
9. Ahmed N, Brawley V, Hegde M, Bielamowicz K, Kalra M, Landi D, et al. HER2-Specific Chimeric Antigen Receptor-Modified Virus-Specific T Cells for Progressive Glioblastoma: A Phase 1 Dose-Escalation Trial. JAMA Oncol 2017; 3(8):1094-101 doi 10.1001/jamaoncol.2017.0184.
10. O'Rourke D M, Nasrallah M P, Desai A, Melenhorst J J, Mansfield K, Morrissette J J D, et al. A single dose of peripherally infused EGFRvIII-directed CAR T cells mediates antigen loss and induces adaptive resistance in patients with recurrent glioblastoma. Sci Transl Med 2017; 9(399) doi 10.1126/scitranslmed.aaa0984.
11. Akhavan D, Alizadeh D, Wang D, Weist M R, Shepphird J K, Brown C E. CAR T cells for brain tumors: Lessons learned and road ahead. Immunological reviews 2019; 290(1):60-84 doi 10.1111/imr.12773.
12. Chuntova P, Downey K M, Hegde B, Almeida N D, Okada H. Genetically Engineered T-Cells for Malignant Glioma: Overcoming the Barriers to Effective Immunotherapy. Front Immunol 2018; 9:3062 doi 10.3389/fimmu.2018.03062.
13. Lim W A, June C H. The Principles of Engineering Immune Cells to Treat Cancer. Cell 2017; 168(4):724-40 doi 10.1016/j.cell.2017.01.016.
14. Simeonov D R, Marson A. CRISPR-Based Tools in Immunity. Annual review of immunology 2019; 37:571-97 doi 10.1146/annurev-immunol-042718-041522.
15. Stadtmauer E A, Fraietta J A, Davis M M, Cohen A D, Weber K L, Lancaster E, et al. CRISPR-engineered T cells in patients with refractory cancer. Science (New York, N Y) 2020; 367(6481) doi 10.1126/science.aba7365.
16. Tang N, Cheng C, Zhang X, Qiao M, Li N, Mu W, et al. TGF-beta inhibition via CRISPR promotes the long-term efficacy of CAR T cells against solid tumors. JCI Insight 2020; 5(4) doi 10.1172/jci.insight.133977.
17. Crowther M D, Dolton G, Legut M, Caillaud M E, Lloyd A, Attaf M, et al. Genome-wide CRISPR-Cas9 screening reveals ubiquitous T cell cancer targeting via the monomorphic MHC class I-related protein MR1. Nature immunology 2020; 21(2):178-85 doi 10.1038/s41590-019-0578-8.
18. Manguso R T, Pope H W, Zimmer M D, Brown F D, Yates K B, Miller B C, et al. In vivo CRISPR screening identifies Ptpn2 as a cancer immunotherapy target. Nature 2017; 547(7664):413-8 doi 10.1038/nature23270.
19. Patel S J, Sanjana N E, Kishton R J, Eidizadeh A, Vodnala S K, Cam M, et al. Identification of essential genes for cancer immunotherapy. Nature 2017; 548(7669):537-42 doi 10.1038/nature23477.
20. Dong M B, Wang G, Chow R D, Ye L, Zhu L, Dai X, et al. Systematic Immunotherapy Target Discovery Using Genome-Scale In Vivo CRISPR Screens in CD8 T Cells. Cell 2019; 178(5):1189-204 e23 doi 10.1016/j.cell.2019.07.044.
21. Shifrut E, Carnevale J, Tobin V, Roth T L, Woo J M, Bui C T, et al. Genome-wide CRISPR Screens in Primary Human T Cells Reveal Key Regulators of Immune Function. Cell 2018; 175(7):1958-71 e15 doi 10.1016/j.cell.2018.10.024.
22. Wei J, Long L, Zheng W, Dhungana Y, Lim S A, Guy C, et al. Targeting REGNASE-1 programs long-lived effector T cells for cancer therapy. Nature 2019; 576(7787):471-6 doi 10.1038/s41586-019-1821-z.
23. Harris D T, Hager M V, Smith S N, Cai Q, Stone J D, Kruger P, et al. Comparison of T Cell Activities Mediated by Human TCRs and CARs That Use the Same Recognition Domains. J Immunol 2018; 200(3):1088-100 doi 10.4049/jimmunol.1700236.
24. Lathia J D, Mack S C, Mulkearns-Hubert E E, Valentim C L, Rich J N. Cancer stem cells in glioblastoma. Genes & development 2015; 29(12):1203-17 doi 10.1101/gad.261982.115.
25. Prager B C, Xie Q, Bao S, Rich J N. Cancer Stem Cells: The Architects of the Tumor Ecosystem. Cell Stem Cell 2019; 24(1):41-53 doi 10.1016/j.stem.2018.12.009.
26. Brown C E, Aguilar B, Starr R, Yang X, Chang W C, Weng L, et al. Optimization of IL13Ralpha2-Targeted Chimeric Antigen Receptor T Cells for Improved Anti-tumor Efficacy against Glioblastoma. Molecular therapy: the journal of the American Society of Gene Therapy 2018; 26(1):31-44 doi 10.1016/j.ymthe.2017.10.002.
27. Fraietta J A, Lacey S F, Orlando E J, Pruteanu-Malinici I, Gohil M, Lundh S, et al. Determinants of response and resistance to CD19 chimeric antigen receptor (CAR) T cell therapy of chronic lymphocytic leukemia. Nat Med 2018; 24(5):563-71 doi 10.1038/s41591-018-0010-1.
28. Rossi J, Paczkowski P, Shen Y W, Morse K, Flynn B, Kaiser A, et al. Preinfusion polyfunctional anti-CD19 chimeric antigen receptor T cells are associated with clinical outcomes in NHL. Blood 2018; 132(8):804-14 doi 10.1182/blood-2018-01-828343.
29. Singh N, Lee Y G, Shestova O, Ravikumar P, Hayer K E, Hong S J, et al. Impaired Death Receptor Signaling in Leukemia Causes Antigen-Independent Resistance by Inducing CAR T-cell Dysfunction. Cancer discovery 2020; 10(4):552-67 doi 10.1158/2159-8290. CD-19-0813.
30. Wang D, Aguilar B, Starr R, Alizadeh D, Brito A, Sarkissian A, et al. Glioblastoma-targeted CD4+ CAR T cells mediate superior antitumor activity. JCI insight 2018; 3(10):e99048 doi 10.1172/jci.insight.99048.
31. Wang D, Starr R, Alizadeh D, Yang X, Forman S J, Brown C E. In Vitro Tumor Cell Rechallenge For Predictive Evaluation of Chimeric Antigen Receptor T Cell Antitumor Function. Journal of visualized experiments: JoVE 2019(144) doi 10.3791/59275.
32. Doench J G, Fusi N, Sullender M, Hegde M, Vaimberg E W, Donovan K F, et al. Optimized sgRNA design to maximize activity and minimize off-target effects of CRISPR-Cas9. Nature biotechnology 2016; 34(2):184-91 doi 10.1038/nbt.3437.
33. Brown C E, Warden C D, Starr R, Deng X, Badie B, Yuan Y C, et al. Glioma IL13Ralpha2 is associated with mesenchymal signature gene expression and poor patient prognosis. PLoS One 2013; 8(10):e77769 doi 10.1371/journal.pone.0077769.
34. Silva-Filho J L, Caruso-Neves C, Pinheiro AAS. IL-4: an important cytokine in determining the fate of T cells. Biophys Rev 2014; 6(1):111-8 doi 10.1007/s12551-013-0133-z.
35. Crawford A, Angelosanto J M, Kao C, Doering T A, Odorizzi P M, Barnett B E, et al. Molecular and transcriptional basis of CD4(+) T cell dysfunction during chronic infection. Immunity 2014; 40(2):289-302 doi 10.1016/j.immuni.2014.01.005.
36. Martinez G J, Pereira R M, Aijo T, Kim E Y, Marangoni F, Pipkin M E, et al. The transcription factor NFAT promotes exhaustion of activated CD8(+) T cells. Immunity 2015; 42(2):265-78 doi 10.1016/j.immuni.2015.01.006.
37. Khan O, Giles J R, McDonald S, Manne S, Ngiow S F, Patel K P, et al. TOX transcriptionally and epigenetically programs CD8(+) T cell exhaustion. Nature 2019; 571(7764):211-8 doi 10.1038/s41586-019-1325-x.
38. Dong Y, Li X, Yu Y, Lv F, Chen Y. JAK/STAT signaling is involved in IL-35-induced inhibition of hepatitis B virus antigen-specific cytotoxic T cell exhaustion in chronic hepatitis B. Life Sci 2020; 252:117663 doi 10.1016/j.Ifs.2020.117663.
39. Ma X, Bi E, Lu Y, Su P, Huang C, Liu L, et al. Cholesterol Induces CD8(+) T Cell Exhaustion in the Tumor Microenvironment. Cell metabolism 2019; 30(1):143-56 e5 doi 10.1016/j.cmet.2019.04.002.
40. Chen J, Lopez-Moyado I F, Seo H, Lio C J, Hempleman L J, Sekiya T, et al. NR4A transcription factors limit CAR T cell function in solid tumours. Nature 2019; 567(7749):530-4 doi 10.1038/s41586-019-0985-x.
41. Singh U, Shamran H, Singh N, Guan H B, Mishra M, Price R L, et al. Blocking fatty acid amide hydrolase reduces T cell activation and attenuates experimental colitis. Journal of Immunology 2015; 194.
42. Schmitz M L, Krappmann D. Controlling N F-kappa B activation in T cells by costimulatory receptors. Cell Death and Differentiation 2006; 13(5):834-42 doi 10.1038/sj.cdd.4401845.
43. Eyquem J, Mansilla-Soto J, Giavridis T, van der Stegen S J, Hamieh M, Cunanan K M, et al. Targeting a CAR to the TRAC locus with CRISPR/Cas9 enhances tumour rejection. Nature 2017; 543(7643):113-7 doi 10.1038/nature21405.
44. Wang D, Starr R, Chang W C, Aguilar B, Alizadeh D, Wright S L, et al. Chlorotoxin-directed CAR T cells for specific and effective targeting of glioblastoma. Sci Transl Med 2020; 12(533) doi 10.1126/scitranslmed.aaw2672.
45. Bandyopadhyay S, Valdor R, Macian F. Tle4 regulates epigenetic silencing of gamma interferon expression during effector T helper cell tolerance. Molecular and cellular biology 2014; 34(2):233-45 doi 10.1128/MCB.00902-13.
46. Naluyima P, Lal K G, Costanzo M C, Kijak G H, Gonzalez V D, Blom K, et al. Terminal Effector CD8 T Cells Defined by an IKZF2(+)IL-7R(−) Transcriptional Signature Express FcgammaRIIIA, Expand in HIV Infection, and Mediate Potent HIV-Specific Antibody-Dependent Cellular Cytotoxicity. J Immunol 2019; 203(8):2210-21 doi 10.4049/jimmunol.1900422.
47. Sowell R T, Kaech S M. Probing the Diversity of T Cell Dysfunction in Cancer. Cell 2016; 166(6):1362-4 doi 10.1016/j.cell.2016.08.058.
48. Shin H J, Lee J B, Park S H, Chang J, Lee C W. T-bet expression is regulated by EGR1-mediated signaling in activated T cells. Clin Immunol 2009; 131(3):385-94 doi 10.1016/j.clim.2009.02.009.
49. Ananieva E A, Patel C H, Drake C H, Powell J D, Hutson S M. Cytosolic branched chain aminotransferase (BCATc) regulates mTORC1 signaling and glycolytic metabolism in CD4+ T cells. The Journal of biological chemistry 2014; 289(27):18793-804 doi 10.1074/jbc.M114.554113.
50. Hess C, Means T K, Autissier P, Woodberry T, Altfeld M, Addo M M, et al. IL-8 responsiveness defines a subset of CD8 T cells poised to kill. Blood 2004; 104(12):3463-71 doi 10.1182/blood-2004-03-1067.
51. Trifilo M J, Bergmann C C, Kuziel W A, Lane T E. C C chemokine ligand 3 (CCL3) regulates CD8(+)-T-cell effector function and migration following viral infection. Journal of virology 2003; 77(7):4004-14 doi 10.1128/jvi.77.7.4004-4014.2003.
52. Kiniry B E, Hunt P W, Hecht F M, Somsouk M, Deeks S G, Shacklett B L. Differential Expression of CD8(+) T Cell Cytotoxic Effector Molecules in Blood and Gastrointestinal Mucosa in HIV-1 Infection. J Immunol 2018; 200(5):1876-88 doi 10.4049/jimmunol.1701532.
53. Duraiswamy J, Ibegbu C C, Masopust D, Miller J D, Araki K, Doho G H, et al. Phenotype, function, and gene expression profiles of programmed death-1(hi) CD8 T cells in healthy human adults. J Immunol 2011; 186(7):4200-12 doi 10.4049/jimmunol.1001783.
54. Wherry E J, Ha S J, Kaech S M, Haining W N, Sarkar S, Kalia V, et al. Molecular signature of CD8+ T cell exhaustion during chronic viral infection. Immunity 2007; 27(4):670-84 doi 10.1016/j.immuni.2007.09.006.
55. Lynn R C, Weber E W, Sotillo E, Gennert D, Xu P, Good Z, et al. c-Jun overexpression in CAR T cells induces exhaustion resistance. Nature 2019; 576(7786):293-300 doi 10.1038/s41586-019-1805-z.
56. Muller M R, Rao A. NFAT, immunity and cancer: a transcription factor comes of age. Nat Rev Immunol 2010; 10(9):645-56 doi 10.1038/nri2818.
57. Myers L M, Tal M C, Torrez Dulgeroff L B, Carmody A B, Messer R J, Gulati G, et al. A functional subset of CD8(+) T cells during chronic exhaustion is defined by SIRPalpha expression. Nature communications 2019; 10(1):794 doi 10.1038/s41467-019-08637-9.
58. Maimela N R, Liu S, Zhang Y. Fates of CD8+ T cells in Tumor Microenvironment. Comput Struct Biotechnol J 2019; 17:1-13 doi 10.1016/j.csbj.2018.11.004.
59. Bhairavabhotla R, Kim Y C, Glass D D, Escobar™, Patel M C, Zahr R, et al. Transcriptome profiling of human FoxP3+ regulatory T cells. Hum Immunol 2016; 77(2):201-13 doi 10.1016/j.humimm.2015.12.004.
60. Nishio H, Yaguchi T, Sugiyama J, Sumimoto H, Umezawa K, Iwata T, et al. Immunosuppression through constitutively activated N F-kappa B signalling in human ovarian cancer and its reversal by an N F-kappa B inhibitor. British Journal of Cancer 2014; 110(12):2965-74 doi 10.1038/bjc.2014.251.
61. Yamamoto Y, Gaynor R B. Therapeutic potential of inhibition of the N F-kappaB pathway in the treatment of inflammation and cancer. The Journal of clinical investigation 2001; 107(2):135-42 doi 10.1172/JC111914.
62. Liao W, Overman M J, Boutin A T, Shang X, Zhao D, Dey P, et al. KRAS-IRF2 Axis Drives Immune Suppression and Immune Therapy Resistance in Colorectal Cancer. Cancer Cell 2019; 35(4):559-72 e7 doi 10.1016/j.ccell.2019.02.008.
63. Li W M, Liu H R. CCL20-CCR6 Cytokine Network Facilitate Treg Activity in Advanced Grades and Metastatic Variants of Hepatocellular Carcinoma. Scand J Immunol 2016; 83(1):33-7 doi 10.1111/sji.12367.
64. Do Y, Nagarkatti P S, Nagarkatti M. Role of CD44 and hyaluronic acid (H A) in activation of alloreactive and antigen-specific T cells by bone marrow-derived dendritic cells. Journal of immunotherapy (Hagerstown, Md: 1997) 2004; 27(1):1-12 doi 10.1097/00002371-200401000-00001.
65. Jankowska K I, Williamson E K, Roy N H, Blumenthal D, Chandra V, Baumgart T, et al. Integrins Modulate T Cell Receptor Signaling by Constraining Actin Flow at the Immunological Synapse. Frontiers in Immunology 2018; 9 doi ARTN 2510.3389/fimmu.2018.00025.
66. Brown C E, Alizadeh D, Starr R, Weng L, Wagner J R, Naranjo A, et al. Regression of Glioblastoma after Chimeric Antigen Receptor T-Cell Therapy. N Engl J Med 2016; 375(26):2561-9 doi 10.1056/NEJMoa1610497.
67. Batlle E, Massague J. Transforming Growth Factor-beta Signaling in Immunity and Cancer. Immunity 2019; 50(4):924-40 doi 10.1016/j.immuni.2019.03.024.
68. Deng Q, Han G, Puebla-Osorio N, Ma MCJ, Strati P, Chasen B, et al. Characteristics of anti-CD19 CAR T cell infusion products associated with efficacy and toxicity in patients with large B cell lymphomas. Nat Med 2020 doi 10.1038/s41591-020-1061-7.
69. Ghassemi S, Nunez-Cruz S, O'Connor R S, Fraietta J A, Patel P R, Scholler J, et al. Reducing Ex Vivo Culture Improves the Antileukemic Activity of Chimeric Antigen Receptor (CAR) T Cells. Cancer Immunol Res 2018; 6(9):1100-9 doi 10.1158/2326-6066.CIR-17-0405.
70. Mount C W, Majzner R G, Sundaresh S, Arnold E P, Kadapakkam M, Haile S, et al. Potent antitumor efficacy of anti-GD2 CAR T cells in H3-K27M+ diffuse midline gliomas. Nature Medicine 2018; 24(5):572-9 doi 10.1038/s41591-018-0006-x.
71. Theruvath J, Sotillo E, Mount C W, Graef C M, Delaidelli A, Heitzeneder S, et al. Locoregionally administered B7-H3-targeted CAR T cells for treatment of atypical teratoid/rhabdoid tumors. Nat Med 2020; 26(5):712-9 doi 10.1038/s41591-020-0821-8.
72. Donovan L K, Delaidelli A, Joseph S K, Bielamowicz K, Fousek K, Holgado B L, et al. Locoregional delivery of CAR T cells to the cerebrospinal fluid for treatment of metastatic medulloblastoma and ependymoma. Nat Med 2020; 26(5):720-31 doi 10.1038/s41591-020-0827-2.
73. Ye L, Park J J, Dong M B, Yang Q, Chow R D, Peng L, et al. In vivo CRISPR screening in CD8 T cells with AAV-Sleeping Beauty hybrid vectors identifies membrane targets for improving immunotherapy for glioblastoma. Nature biotechnology 2019; 37(11):1302-13 doi 10.1038/s41587-019-0246-4.
74. Arvanitis C D, Ferraro G B, Jain R K. The blood-brain barrier and blood-tumour barrier in brain tumours and metastases. Nat Rev Cancer 2020; 20(1):26-41 doi 10.1038/s41568-019-0205-x.
75. Platt R J, Chen S, Zhou Y, Yim M J, Swiech L, Kempton H R, et al. CRISPR-Cas9 knockin mice for genome editing and cancer modeling. Cell 2014; 159(2):440-55 doi 10.1016/j.cell.2014.09.014.
76. Henriksson J, Chen X, Gomes T, Ullah U, Meyer K B, Miragaia R, et al. Genome-wide CRISPR Screens in T Helper Cells Reveal Pervasive Crosstalk between Activation and Differentiation. Cell 2019; 176(4):882-96 e18 doi 10.1016/j.cell.2018.11.044.
77. Brown C E, Mackall C L. CAR T cell therapy: inroads to response and resistance. Nat Rev Immunol 2019; 19(2):73-4 doi 10.1038/s41577-018-0119-y.
78. Wherry E J, Kurachi M. Molecular and cellular insights into T cell exhaustion. Nat Rev Immunol 2015; 15(8):486-99 doi 10.1038/nri3862.
79. Long A H, Haso W M, Shern J F, Wanhainen K M, Murgai M, Ingaramo M, et al. 4-1 B B costimulation ameliorates T cell exhaustion induced by tonic signaling of chimeric antigen receptors. Nat Med 2015; 21(6):581-90 doi 10.1038/nm.3838.
80. Cherkassky L, Morello A, Villena-Vargas J, Feng Y, Dimitrov D S, Jones D R, et al. Human CAR T cells with cell-intrinsic PD-1 checkpoint blockade resist tumor-mediated inhibition. The Journal of clinical investigation 2016; 126(8):3130-44 doi 10.1172/JC183092.
81. Rafiq S, Yeku 00, Jackson H J, Purdon T J, van Leeuwen D G, Drakes D J, et al. Targeted delivery of a PD-1-blocking scFv by CAR-T cells enhances anti-tumor efficacy in vivo. Nature biotechnology 2018; 36(9):847-56 doi 10.1038/nbt.4195.
82. Schietinger A, Philip M, Krisnawan V E, Chiu E Y, Delrow J J, Basom R S, et al. Tumor-Specific T Cell Dysfunction Is a Dynamic Antigen-Driven Differentiation Program Initiated Early during Tumorigenesis. Immunity 2016; 45(2):389-401 doi 10.1016/j.immuni.2016.07.011.
83. Man K, Gabriel S S, Liao Y, Gloury R, Preston S, Henstridge D C, et al. Transcription Factor IRF4 Promotes CD8(+) T Cell Exhaustion and Limits the Development of Memory-like T Cells during Chronic Infection. Immunity 2017; 47(6):1129-41 e5 doi 10.1016/j.immuni.2017.11.021.
84. Li P, Spolski R, Liao W, Wang L, Murphy T L, Murphy K M, et al. BATF-JUN is critical for IRF4-mediated transcription in T cells. Nature 2012; 490(7421):543-6 doi 10.1038/naturel1530.
85. Meixner A, Karreth F, Kenner L, Wagner E F. JunD regulates lymphocyte proliferation and T helper cell cytokine expression. The EMBO journal 2004; 23(6):1325-35 doi 10.1038/sj.emboj.7600133.
86. Chiu R, Angel P, Karin M. Jun-B differs in its biological properties from, and is a negative regulator of, c-Jun. Cell 1989; 59(6):979-86 doi 10.1016/0092-8674(89)90754-x.
87. Wei F, Zhong S, Ma Z, Kong H, Medvec A, Ahmed R, et al. Strength of PD-1 signaling differentially affects T-cell effector functions. Proc Natl Acad Sci USA 2013; 110(27):E2480-9 doi 10.1073/pnas.1305394110.
88. Jiao S, Subudhi S K, Aparicio A, Ge Z, Guan B, Miura Y, et al. Differences in Tumor Microenvironment Dictate T Helper Lineage Polarization and Response to Immune Checkpoint Therapy. Cell 2019; 179(5):1177-90 e13 doi 10.1016/j.cell.2019.10.029.
89. Tran E, Turcotte S, Gros A, Robbins P F, Lu Y C, Dudley M E, et al. Cancer immunotherapy based on mutation-specific CD4+ T cells in a patient with epithelial cancer. Science (New York, N Y) 2014; 344(6184):641-5 doi 10.1126/science.1251102.
90. Lee D W, Kochenderfer J N, Stetler-Stevenson M, Cui Y K, Delbrook C, Feldman S A, et al. T cells expressing CD19 chimeric antigen receptors for acute lymphoblastic leukaemia in children and young adults: a phase 1 dose-escalation trial. Lancet 2015; 385(9967):517-28 doi 10.1016/S0140-6736(14)61403-3.
91. Bao S, Wu Q, McLendon R E, Hao Y, Shi Q, Hjelmeland A B, et al. Glioma stem cells promote radioresistance by preferential activation of the DNA damage response. Nature 2006; 444(7120):756-60 doi 10.1038/nature05236.
92. Xie Q, Wu T P, Gimple R C, Li Z, Prager B C, Wu Q, et al. N(6)-methyladenine DNA Modification in Glioblastoma. Cell 2018; 175(5):1228-43 e20 doi 10.1016/j.cell.2018.10.006.
93. Priceman S J, Tilakawardane D, Jeang B, Aguilar B, Murad J P, Park A K, et al. Regional Delivery of Chimeric Antigen Receptor-Engineered T Cells Effectively Targets HER2(+) Breast Cancer Metastasis to the Brain. Clin Cancer Res 2018; 24(1):95-105 doi 10.1158/1078-0432.ccr-17-2041.
94. Hanzelmann S, Castelo R, Guinney J. GSVA: gene set variation analysis for microarray and RNA-seq data. BMC Bioinformatics 2013; 14:7 doi 10.1186/1471-2105-14-7.
95. Stuart T, Butler A, Hoffman P, Hafemeister C, Papalexi E, Mauck W M, 3rd, et al. Comprehensive Integration of Single-Cell Data. Cell 2019; 177(7):1888-902 e21 doi 10.1016/j.cell.2019.05.031.
96. Brown C E, Starr R, Martinez C, Aguilar B, D'Apuzzo M, Todorov I, et al. Recognition and killing of brain tumor stem-like initiating cells by CD8+ cytolytic T cells. Cancer Res 2009; 69(23):8886-93 doi 10.1158/0008-5472.CAN-09-2687.
97. Thorsson V, Gibbs D L, Brown S D, Wolf D, Bortone D S, Ou Yang T H, et al. The Immune Landscape of Cancer. Immunity 2018; 48(4):812-30 e14 doi 10.1016/j.immuni.2018.03.023.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A population of engineered human T cells, wherein the engineered human T cells comprise: a disrupted Transducin-Like Enhancer of Split 4 (TLE4) gene, a disrupted Transmembrane Protein 184B (MEM184B) gene, a disrupted Eukaryotic Translation Initiation Factor 5A-1 (EIF5A) gene or a disrupted Ikaros Family Zinc Finger Protein 2 (IKZF2) gene.

2. The population of engineered human T cells of claim 1, comprising a disrupted TLE4 gene.

3. The population of engineered human T cells of claim 1, comprising a disrupted MEM184B gene.

4. The population of engineered human T cells of claim 1, comprising a disrupted EIF5A gene.

5. The population of engineered human T cells of claim 1, comprising a disrupted IKZF2 gene.

6. The population of engineered human T cells of claim 2, wherein the disrupted TLE4 gene comprises an insertion of at least 10 contiguous nucleotides into SEQ ID NO: D1.

7. The population of engineered human T cells of claim 3, wherein the disrupted MEM184B gene comprises an insertion of at least 10 contiguous nucleotides into SEQ ID NO: D2.

8. The population of engineered human T cells of claim 3, wherein the disrupted EIF5A gene comprises an insertion of at least 10 contiguous nucleotides into SEQ ID NO: D3.

9. The population of engineered human T cells of claim 3, wherein the disrupted IKZF2 gene comprises a deletion of at least 10 contiguous nucleotides of SEQ ID NO: D4

10. The population of engineered human T cells of claim 2, wherein the disrupted TLE4 gene comprises a deletion of at least 10 contiguous nucleotides of SEQ ID NO: D1.

11. The population of engineered human T cells of claim 3, wherein the disrupted MEM184B gene comprises a deletion of at least 10 contiguous nucleotides of SEQ ID NO: D2.

12. The population of engineered human T cells of claim 3, wherein the disrupted EIF5A gene comprises a deletion of at least 10 contiguous nucleotides of SEQ ID NO: D3.

13. The population of engineered human T cells of claim 3, wherein the disrupted IKZF2 gene comprises a deletion of at least 10 contiguous nucleotides of SEQ ID NO: D4.

14. The population of engineered T cells of any of claims 2-5, wherein the disrupted gene is disrupted by a nucleic acid encoding a chimeric antigen receptor.

15. The population of engineered human T cells of claim 1, wherein at least 30% of the T cells comprises a nucleic acid molecule comprising a nucleotide sequence encoding a chimeric antigen receptor (CAR) wherein the chimeric antigen receptor comprises a targeting domain, a spacer, a transmembrane domain, a co-stimulatory domain, and a CD3 (signaling domain.

16. The population of engineered human T cells of claim 15, wherein the targeting domain comprises a scFv that selectively binds a tumor cell antigen.

17. The population of engineered human T cells of claim 15, wherein the targeting domain comprises a ligand for a cell surface receptor.

18. The population of engineered T cells of claim 15, wherein the nucleic acid molecule encoding the CAR is an mRNA.

19. The population of T cells of claim 1 wherein at least 75%, at least 80%, at least 85%, at least 90%, or at least 95% of the engineered T cells do not express a detectable level of TLE4.

20. The population of T cells of claim 1 wherein at least 75%, at least 80%, at least 85%, at least 90%, or at least 95% of the engineered T cells do not express a detectable level of MEM184B.

21. The population of T cells of claim 1 wherein at least 75%, at least 80%, at least 85%, at least 90%, or at least 95% of the engineered T cells do not express a detectable level of EIF5A.

22. The population of T cells of claim 1 wherein at least 75%, at least 80%, at least 85%, at least 90%, or at least 95% of the engineered T cells do not express a detectable level of KZF2.

23. A method for producing an engineered T cell, the method comprising
(a) delivering to a T cell:
a RNA-guided nuclease,
a gRNA targeting a TLE4 gene, a EMM1848 gene, or a KZF2 gene,
a vector comprising a donor template that comprises a nucleic acid encoding a CAR; and
(b) producing an engineered T cell suitable for allogeneic transplantation.
